SEQUENCE LISTING (8 sequences)

SEQ ID NO: 1 - DNA, 1683 bp, Homo sapiens
  misc_feature (25)..(1626): CDS for LP276
agctgtcagccgcctcacaggaagatgctgcgtcggcggggcagccctggcatgggtgtgcatgtgggtgcagccctgggagcactgtggttctgcctcacaggagccctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcatcctgcgggtggtgctgggtgcaaatggcacctacagctgcctggtgcgcaaccccgtgctgcagcaggatgcgcacagctctgtcaccatcacaccccagagaagccccacaggagccgtggaggtccaggtccctgaggacccggtggtggccctagtgggcaccgatgccaccctgcgctgctccttctcccccgagcctggcttcagcctggcacagctcaacctcatctggcagctgacagacaccaaacagctggtgcacagtttcaccgaaggccgggaccagggcagcgcctatgccaaccgcacggccctcttcccggacctgctggcacaaggcaatgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccggggctaccctgaggctgaggtgttctggcaggatgggcagggtgtgcccctgactggcaacgtgaccacgtcgcagatggccaacgagcagggcttgtttgatgtgcacagcgtcctgcgggtggtgctgggtgcgaatggcacctacagctgcctggtgcgcaaccccgtgctgcagcaggatgcgcacggctctgtcaccatcacagggcagcctatgacattccccccagaggccctgtgggtgaccgtggggctgtctgtctgtctcattgcactgctggtggccctggctttcgtgtgctggagaaagatcaaacagagctgtgaggaggagaatgcaggagctgaggaccaggatggggagggagaaggctccaagacagccctgcagcctctgaaacactctgacagcaaagaagatgatggacaagaaatagcctgaccatgaggaccagggagctgctacccctccctacagctcctaccctctggctgc

SEQ ID NO: 2 - protein, 534 aa, Homo sapiens
  MISC_FEATURE (29)..(465): Extracellular domain
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 3 - DNA, 2151 bp, Homo sapiens
  misc_feature (1)..(2151): CDS for LP276ATFV
atgctgcgtcggcggggcagccctggcatgggtgtgcatgtgggtgcagccctgggagcactgtggttctgcctcacaggagccctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcatcctgcgggtggtgctgggtgcaaatggcacctacagctgcctggtgcgcaaccccgtgctgcagcaggatgcgcacagctctgtcaccatcacaccccagagaagccccacaggagccgtggaggtccaggtccctgaggacccggtggtggccctagtgggcaccgatgccaccctgcgctgctccttctcccccgagcctggcttcagcctggcacagctcaacctcatctggcagctgacagacaccaaacagctggtgcacagtttcaccgaaggccgggaccagggcagcgcctatgccaaccgcacggccctcttcccggacctgctggcacaaggcaatgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccggggctaccctgaggctgaggtgttctggcaggatgggcagggtgtgcccctgactggcaacgtgaccacgtcgcagatggccaacgagcagggcttgtttgatgtgcacagcgtcctgcgggtggtgctgggtgcgaatggcacctacagctgcctggtgcgcaaccccgtgctgcagcaggatgcgcacggctctgtcaccatcacagggcagcctatgacattccccccagaggatatcgagcccaaatcttgtgacaaaactcacacatgcccaccgtgcccagcacctgagctcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggagtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaaggatcgactacaaggatgacgacgacaagcacgtgcatcaccatcaccatcac

SEQ ID NO: 4 - protein, 717 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEDIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQEYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRIDYKDDDDKHVHHHHHH

SEQ ID NO: 5 - DNA, 1026 bp, Homo sapiens
  misc_feature (25)..(969): CDS for LP276S
agctgtcagccgcctcacaggaagatgctgcgtcggcggggcagccctggcatgggtgtgcatgtgggtgcagccctgggagcactgtggttctgcctcacaggagccctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcatcctgcgggtggtgctgggtgcaaatggcacctacagctgcctggtgcgcaaccccgtgctgcagcaggatgcgcacagctctgtcaccatcacaccccagagaagccccacaggagccgtggaggtccaggtcgtggggctgtctgtctgtctcattgcactgctggtggccctggctttcgtgtgctggagaaagatcaaacagagctgtgaggaggagaatgcaggagctgaggaccaggatggggagggagaaggctccaagacagccctgcagcctctgaaacactctgacagcaaagaagatgatggacaagaaatagcctgaccatgaggaccagggagctgctacccctccctacagctcctaccctctggctgc

SEQ ID NO: 6 - protein, 315 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVVGLSVCLIALLVALAFVCWRKIKQSCEEENAGAEDQDGEGEGSKTALQPLKHSDSKEDDGQEIA

SEQ ID NO: 7 - DNA, 1509 bp, Homo sapiens
  misc_feature (1)..(1509): CDS for LP276ATFV2
atgctgcgtcggcggggcagccctggcatgggtgtgcatgtgggtgcagccctgggagcactgtggttctgcctcacaggagccctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcatcctgcgggtggtgctgggtgcaaatggcacctacagctgcctggtgcgcaaccccgtgctgcagcaggatgcgcacagctctgtcaccatcacaccccagagaagccccacaggagccgtggaggtccaggtcgatatcgagcccaaatcttgtgacaaaactcacacatgcccaccgtgcccagcacctgagctcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggagtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaaggatcgactacaaggatgacgacgacaagcacgtgcatcaccatcaccatcac

SEQ ID NO: 8 - protein, 503 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVDIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQEYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRIDYKDDDDKHVHHHHHH

(19) United States
(12) Patent Application Publication
(10) Pub. No.: US 2004/0236088 A1
(43) Pub. Date: Nov. 25, 2004

(54) Novel polypeptide analogs and fusions and their methods of use

(76) Inventors: Josef Georg Heuer, Indianapolis, IN (US); Songqing Na, Carmel, IN (US); Angela Jeannine Okragly, Indianapolis, IN (US); Weijia Ou, Fishers, IN (US)

Correspondence Address: ELI LILLY AND COMPANY, PATENT DIVISION, P.O. BOX 6288, INDIANAPOLIS, IN, 46206-6288, US

(21) Appl. No.: 10/484,122

(22) PCT Filed: Jul. 24, 2002
(86) PCT No.: PCT/US02/21293

Related U.S. Application Data
(60) Provisional application No. 60/309,674, filed on Aug. 2, 2001.

(51) Int. Cl. C07H 21/04

(52) U.S. Cl. 536/23.2; 435/69.1; 435/320.1; 435/325; 530/388.1; 530/350; 424/94.6

(57) ABSTRACT

Novel polypeptide analogs and fusion proteins of a transmembrane protein, LP276, are provided. Vectors and host cells directed to these polypeptides are provided. Additionally, methods of use are provided for the treatment or prevention of allergic autoimmune diseases, type 1 diabetes, inflammation, immunodeficiencies, cancers, and infectious diseases by administering an LP276 polypeptide, analogs and fusion proteins thereof to a patient in need of such therapy.

The present invention relates to novel analogs of a transmembrane protein (LP276), designated herein as LP276L and LP276S, fusion proteins made with each analog (designated herein as LP276ATFV and LP276ATFV2, respectively), vectors and host cells directed to these polypeptides. The invention also provides methods of use in the treatment or prevention of sepsis, gram negative bacteremia, allergic responses, allergic autoimmune diseases, type 1 diabetes, Th1-dependent insulitis, inflammation, multiple sclerosis, rheumatoid arthritis, inflammatory bowel disease, liver failure, ARDS, immunodeficiencies, cancers, infectious diseases, and conditions or symptoms related thereto by administering an LP276 polypeptide, analog, or fusion to a patient in need of such therapy.

Lipopolysaccharide (“LPS”), a known endotoxin, is a component of the outer membrane of gram negative bacteria. In addition, pathogenic bacteria, viruses, and plants liberate lipopolysaccharide-inducing substances. LPS is the major mediator in the development of endotoxin-induced shock.

The chemical structures of LPS molecules obtained from different bacteria may vary in a species-specific fashion. However, the region called the lipid A region is common to all LPS molecules [Rietschel, et al., Handbook of Endotoxins, Elsevier, 1:187-214 (1984)]. The lipid A region mediates many, if not all, of the LPS-dependent pathophysiologic changes that characterize sepsis and gram negative bacteremia. LPS is a primary cause of death in humans afflicted with gram-negative sepsis [van Deventer, et al., Lancet 1(8586):605-9 (1988); Ziegler, et al., J. Infect. Dis. 136(1):19-28 (1987)]. LPS released from gram-negative bacteria infection may also play a role in the pathology of autoimmune conditions such as Reiter's syndrome, which is associated with rheumatoid arthritis.

LPS challenge to polymorphonuclear leukocytes, endothelial cells, and cells of the monocyte/macrophage lineage causes the cells to rapidly release a variety of cell products, including immunoregulatory substances that are capable of initiating, modulating, or mediating humoral and cellular immune responses and processes. LPS induces monocytes/macrophages to release inflammatory cytokines such as TNF-alpha, IL-1, IL-6, and IL-12 which play a major role in the cascade of events leading to endotoxic shock.

Tumor necrosis factor (TNF) appears to be a primary mediator of septic shock [Beutler, et al., N. Eng. J. Med. 316(7):379-85 (1987)]. Intravenous injection of LPS into animals and man produces a rapid, transient release of TNF-alpha [Beutler, et al., Science 229(4716):869-71 (1985); Mathison, et al., J. Clin. Invest. 81(6):1925-37 (1988)].

In addition to TNF-alpha, interferon-gamma (IFN-gamma) and IL-12 also contribute to LPS-induced sepsis [Ozmen, et al., J. Exp. Med. 180(3):907-15 (1994)]. IFN-gamma is secreted by T cells and NK cells. The immunomodulatory effects of IFN-gamma are extensive and diverse. In monocyte/macrophages, the activities of IFN-gamma include: increasing the expression of class I and II MHC antigens; increasing the production of IL-1, platelet-activating factor and hydrogen peroxide; protection of monocytes against LAK cell-mediated lysis; downregulation of IL-8 mRNA expression that is upregulated by IL-2; and, with LPS, induction of nitric oxide production [Billiau and Dijkmans, Biochem. Pharmacol. 40(7):1433-9 (1990); Sen and Lengyel, J. Biol. Chem. 267(8):5017-20 (1992); Gusella, et al., J. Immunol. 151(5):2725-32 (1993); Bulut, et al., Biochem. Biophys. Res. Commun. 195(2):1134-8 (1993)]. IFN-gamma has also been demonstrated to be chemotactic for monocytes but not neutrophils [Issekutz and Issekutz, J. Immunol. 151(4):2105-15 (1993)]. IFN-gamma selectively enhances both IgG2a secretion by LPS-stimulated B cells and IgG3 secretion in T cell independent type 2 antigen-mediated B cell activation [Snapper, et al., J. Exp. Med. 175(5):1367-71 (1992); Snapper, et al., J. Immunol. 140(7):2121-7 (1988)]. It has also been reported to induce its own expression [Halloran, et al., J. Immunol. 148(12):3837-46 (1992)]. IFN-gamma has been shown to upregulate ICAM-1, but not E-selectin or VCAM-1, expression on endothelial cells (Thornhill, et al., Scand. J. Immunol. 38(3):279-86 (1993)]. Moreover, IFN-gamma has been shown to contribute to the Swan reaction induced by gram-negative bacteria [Ogasawara, et al., J. Immunol. 160(7):3522-7 (1998)]. IFN-gamma stimulates macrophages and monocytes to secret TNF-alpha and in turn upregulates TNF-alpha receptor expression.

In contrast to IFN-gamma, IL-12 is produced by macrophages and B-lymphocytes. IL-12 has been shown to have multiple effects on T cells and NK cells [D'Andrea, et al., J. Exp. Med. 176(5):1387-98 (1992); Chan, et al., J. Exp. Med. 173(4):869-79 (1991)].

These effects include stimulation of production of IFN-gamma and TNF by resting and activated T and NK cells, synergizing with other IFN-gamma inducers at both the transcriptional and post-transcriptional levels to induce IFN-gamma gene expression, enhancing the cytotoxic activity of resting NK and T cells, inducing and synergizing with IL-2 in the generation of lymphokine-activated killer (LAK) cells, acting as a co-mitogen to stimulate proliferation of resting T cells, and inducing proliferation of activated T and NK cells [D'Andrea, et al., J. Exp. Med. 176(5):1387-98 (1992)]. Evidence indicates that IL-12, produced by macrophages in response to infectious agents, is a central mediator of the cell-mediated immune response by its actions on the development, proliferation, and activities of Th1 cells [Locksley, Proc. Natl. Acad. Sci. USA 90(13):5879-80 (1993); Trinchieri, Immunol. Today 14(7):335-8 (1993); Scott, Science 260(5107):496-7 (1993); Hseih, et al., Science 260(5107):547-9 (1993)]. These IL-12 activities are antagonized by factors which are associated with the development of uncommitted T helper cells into Th2 cells and mediation of the humoral immune response [e.g., IL-4 and IL-10; Locksley, supra (1993); Trinchieri, supra (1993); Scott, supra (1993); and Hseih, supra (1993)].

In addition to sepsis, both IFN-gamma and IL-12 are upregulated in many other inflammatory diseases. For instance, it has been shown that antibodies against IL-12 can prevent superantigen-induced and spontaneous relapses of experimental autoimmune encephalomyelitis [Constantinescu, et al., J. Immunology 161(9):5097-104 (1998)]. It also has been reported that blocking IFN-gamma production in T cells through the use of anti-IL-18 antibodies can impede the development of experimental autoimmune encephalomyelitis (EAE), a T cell-mediated autoimmune disease of the central nervous system that serves as a model for multiple sclerosis [Zamvil and Steinman, Annu. Rev. Immunol. 8:579-621 (1990)].

Type I diabetes is also considered an autoimmune disease. Using the non-obese diabetic (NOD) mouse as an animal model for type 1 diabetes, the NOD mice are challenged with cyclophosphamide to accelerate diabetes development. Spontaneous mononuclear infiltration of several organs occurs in these mice. This invasion, occurring in the pancreas, is accompanied by a loss of beta cells resulting in insulin deficiency [Signore, et al., Histochemistry 101:263-9 (1995)]. The progression of the disease toward intrainsulitis is associated with an increase in Th1 cells and a subsequent loss of beta cells resulting in insulin deficiency [Shehadeh, et al., J. Autoimmun. 6(3):291-300 (1993); Rothe, et al., Diabetologia 37(11):1154-8 (1994)]. The destructive effects of IFN-gamma produced by these T cells can be alleviated using neutralizing antibodies to IFN-gamma or IL-12 [Debray-Sachs, et al., J. Autoimmun. 4(2):237-48 (1991)].

Synergy between IL-12 and IL-18 is important to the production of IFN-gamma from T cells and NK cells, which sustain inflammation [Micallef, et al., Eur. J. Immunol. 26(7):1647-51 (1996)]. In addition to stimulation of IFN-gamma secretion, IL-12 also increases expression of the IL-18 receptor on Th0 cells and B cells. It has been shown that IL-18 is produced by articular chondrocytes and induces proinflammatory and catabolic responses. Increased production of IL-18 is found in synovium of patients with rheumatoid arthritis [Olee, et al., J. Immunol. 162(2):1096-100 (1999); Yamamura, et al., Arthritis Rheum. 40(9):S274 (1997)].

IL-17 is another known proinflammatory molecule. It is produced by activated T lymphocytes, primarily by memory T cells [Rouvier, et al., J. Immunol. 150(12):5445-56 (1993); Yao, et al., J. Immunol. 155(12):5483-6 (1995); Kennedy, et al., J. Interferon Cytokine Res. 16(8):611-7 (1996); Fossiez, et al., J. Exp. Med. 183(6):2593-603 (1996)]. IL-17 appears to mediate communication between the immune system and the hematopoietic system. IL-17 mediation of T cell communication with the hematopoietic system is suggested by two observations. T cell-derived IL-17 induces fibroblasts to secrete IL-6, IL-8, ICAM-1, and G-CSF, apparently by an NF-kB-mediated mechanism [Yao, et al., Immunity 3(6):811-21 (1995)]. IL-6 in turn promotes development of granulocyte/macrophage colonies, and G-CSF directs development of neutrophils [Fossiez, supra (1996); Ikebuchi, et al., Proc. Natl. Acad. Sci. USA 84(24):9035-9 (1987); Berliner, et al., Blood 85(3):799-803 (1995); Roberts and Metcalf, Exp. Hematol. 22(12):1156-63 (1994); Broxmeyer, J. Exp. Med. 183(6):2411-5 (1996)]. IL-17 also enhances proliferation of partially activated T cells, and it upregulates nitric oxide production in osteoarthritic cartilage [Yao, supra (1995); Attur, et al., Arthritis Rheum. 40(6):1050-3 (1997)].

In contrast to the cytokines mentioned thus far, IL-10 is an anti-inflammatory cytokine. IL-10is a pleiotrophic cytokine that inhibits the production of a number of cytokines (including IL-1, GM-CSF, TNF, IL-6, IL-8, IL-10, IL-12, and IFN-gamma) by activated Th-1 cells, NK cells, and monocyte/macrophages. IL-10 has also been shown to inhibit macrophage cytotoxic activity and to stimulate the proliferation and differentiation of B cells, mast cells, and thymic T cells [Moore, et al., Annu. Rev. Immunol. 11:165-90 (1993); Fiorentino, et al., J. Exp. Med. 170(6):2081-95 (1989); Mosmann, Adv. Immunol. 56:1-26(1994)].

Clearly, cytokines play important roles in many inflammatory and autoimmune diseases. In light of this, a significant amount of effort has been directed at identifying molecules which regulate cytokine activity. Regulation of cytokines that modulate the immune system may provide potential treatment of inflammatory and autoimmune diseases.

The present invention concerns polypeptides including analogs or fusion proteins of a mature LP276 polypeptide, and vectors and host cells directed to these polypeptides. This invention also provides methods for treating inflammatory, autoimmune, immunodeficiency, allergic, and proliferative disorders using polypeptides.

This invention relates generally to methods and therapies for effectively preventing or treating sepsis, gram negative bacteremia, allergic responses, allergic autoimmune diseases, type 1 diabetes, Th1-dependent insulitis, inflammation, multiple sclerosis, rheumatoid arthritis, inflammatory bowel disease, liver failure, ARDS, immunodeficiencies, cancers, infectious diseases, and allergic responses and conditions or symptoms related thereto, by administering LP276, LP276L, LP276S, LP276ATFV, or LP276ATFV2 polypeptides.

It is a further objective of the invention to provide methods and means for intervening in the underlying mechanisms of sepsis, gram negative bacteremia, allergic responses, allergic autoimmune diseases, type 1 diabetes, Th1-dependent insulitis, inflammation, multiple sclerosis, rheumatoid arthritis, inflammatory bowel disease, liver failure, ARDS, immunodeficiencies, cancers, infectious diseases, and conditions or symptoms related thereto by administering LP276, LP276L, LP276S, LP276ATFV, or LP276ATFV2 polypeptides to a patient in need of such intervention. Such methods and means expressly include methods for intervening by inhibiting the action of agents that cause or mediate conditions and symptoms of sepsis, gram negative bacteremia, allergic responses, allergic autoimmune diseases, type 1 diabetes, Th1-dependent insulitis, immunodeficiencies, cancers, inflammation, and/or infectious diseases by administration of LP276, LP276L, LP276S, LP276ATFV, or LP276ATFV2 polypeptides.

Preferred polynucleotides for practicing the present invention are those that encode the full-length LP276 polypeptide as shown in SEQ ID NO:2 or the LP276 polypeptide analog, LP276S, as shown in SEQ ID NO:6. More preferred polynucleotides are those that encode the LP276L polypeptide as represented by amino acids 1 or about 29 through about 465 of SEQ ID NO:2, or those that encode LP276ATFV or LP276ATFV2 fusion proteins, as shown in SEQ ID NO:4 or 8, respectively. Most preferred polynucleotides are those that encode amino acids 29 through 701 of SEQ ID NO:4 or amino acids 29 through 487 of SEQ ID NO:8.

Similarly, preferred polypeptides for practicing the present invention are the full-length LP276 polypeptide as shown in SEQ ID NO:2 or LP276S polypeptide as shown in SEQ ID NO:6. A more preferred polypeptide is LP276L polypeptide as represented by amino acids 1 or about 29 through about 465 of SEQ ID NO:2, or LP276ATFV or LP276ATFV2 fusion proteins as shown in SEQ ID NO:4 or 8, respectively. Most preferred polypeptides are represented by amino acids 29 through 701 of SEQ ID NO:4 or amino acids 29 through 487 of SEQ ID NO:8.

Other features and advantages of the invention for the treatment and/or prevention of sepsis, gram negative bacteremia, allergic responses, allergic autoimmune diseases, inflammation, type 1 diabetes, Th1-dependent insulitis, inflammation, multiple sclerosis, rheumatoid arthritis, inflammatory bowel disease, liver failure, ARDS, immunodeficiencies, cancers, infectious diseases, and conditions or symptoms related thereto immunodeficiencies, cancers, inflammation, and/or infectious diseases, or at least one condition or symptom related thereto, will be further apparent from the following detailed description, from the drawings and tables, and from the claims.

Applicants have identified cDNA clones that encode novel analogs of LP276 polypeptide, designated herein as LP276L polypeptide and LP276S polypeptide, having sequence similarity with the human B7 family of proteins. Additionally, applicants have identified fusion proteins of LP276L and LP276S polypeptide, designated herein as LP276ATFV and LP276ATFV2, respectively. Applicants have also identified vectors and host cells directed to these polypeptides. Furthermore, applicants have identified novel utility for LP276, LP276L, LP276S, LP276ATFV, LP276ATFV2 polypeptides, agonists, or antagonists encoded by LP276, LP276L, LP276S, LP276ATFV, or LP276ATFV2 polynucleotides or variants thereof.

In one embodiment, novel utility is contemplated for LP276 polypeptides comprising the amino acid sequence of the open reading frame encoded by the polynucleotide sequence as shown in SEQ ID NO:1. The isolated nucleic acid comprises DNA consisting of nucleotides 25 or about 109 through about 1625, inclusive, of SEQ ID NO:1.

TABLE 1
SEQ ID NO:1, LP276 polynucleotide.

| | |
|---|---|
| AGCTGTCAGC CGCCTCACAG GAAG ATG CTG CGT CGG CGG GGC AGC CCT GGC | 51 |
| Met Leu Arg Arg Arg Gly Ser Pro Gly | |
| 1 5 | |
| ATG GGT GTG CAT GTG GGT GCA GCC CTG GGA GCA CTG TGG TTC TGC CTC | 99 |
| Met Gly Val His Val Gly Ala Ala Leu Gly Ala Leu Trp Phe Cys Leu | |
| 10 15 20 25 | |
| ACA GGA GCC CTG GAG GTC CAG GTC CCT GAA GAC CCA GTG GTG GCA CTG | 147 |
| Thr Gly Ala Leu Glu Val Gln Val Pro Glu Asp Pro Val Val Ala Leu | |
| 30 35 40 | |
| GTG CCC ACC GAT GCC ACC CTG TGC TGC TCC TTC TCC CCT GAG CCT GGC | 195 |
| Val Gly Thr Asp Ala Thr Leu Cys Cys Ser Phe Ser Pro Glu Pro Gly | |
| 45 50 55 | |
| TTC AGC CTG GCA CAG CTC AAC CTC ATC TGG CAG CTG ACA GAT ACC AAA | 243 |
| Phe Ser Leu Ala Gln Leu Asn Leu Ile Trp Gln Leu Thr Asp Thr Lys | |
| 60 65 70 | |
| CAG CTG GTG CAC AGC TTT GCT GAG GGC CAG GAC CAG GGC AGC GCC TAT | 291 |
| Gln Leu Val His Ser Phe Ala Glu Gly Gln Asp Gln Gly Ser Ala Tyr | |
| 75 80 85 | |
| GCC AAC CGC ACG GCC CTC TTC CCG GAC CTG CTG GCA CAG GGC AAC GCA | 339 |
| Ala Asn Arg Thr Ala Leu Phe Pro Asp Leu Leu Ala Gln Gly Asn Ala | |
| 90 95 100 105 | |
| TCC CTG AGG CTG CAG CGC GTG CGT GTG GCG GAC GAG GGC AGC TTC ACC | 387 |
| Ser Leu Arg Leu Gln Arg Val Arg Val Ala Asp Glu Gly Ser Phe Thr | |
| 110 115 120 | |
| TGC TTC GTG AGC ATC CGG GAT TTC CCC AGC GCT GCC GTC AGC CTG CAG | 435 |
| Cys Phe Val Ser Ile Arg Asp Phe Gly Ser Ala Ala Val Ser Leu Gln | |
| 125 120 135 | |
| GTG GCC GCT CCC TAC TCG AAG CCC AGC ATG ACC CTG GAG CCC AAC AAG | 483 |
| Val Ala Ala Pro Tyr Ser Lys Pro Ser Met Thr Leu Glu Pro Asn Lys | |
| 140 145 150 | |
| CAC CTG CGG CCA GGG GAC ACG GTG ACC ATC ACG TGC TCC AGC TAC CAG | 531 |
| Asp Leu Arg Pro Gly Asp Thr Val Thr Ile Thr Cys Ser Ser Tyr Gln | |
| 155 160 165 | |
| GGC TAC CCT GAG GCT GAG GTG TTC TGG CAG GAT GGG CAG GGT GTG CCC | 579 |
| Gly Tyr Pro Glu Ala Glu Val Phe Trp Gln Asp Gly Gln Gly Val Pro | |
| 170 175 180 185 | |
| CTG ACT GGC AAC GTG ACC ACG TCG CAG ATG GCC AAC GAG CAG GGC TTG | 627 |
| Leu Thr Gly Asn Val Thr Thr Ser Gln Met Ala Asn Glu Gln Gly Leu | |
| 190 195 200 | |
| TTT GAT GTG CAC AGC ATC CTG CGG GTG GTG CTG GGT GCA AAT GGC ACC | 675 |
| Phe Asp Val His Ser Ile Leu Arg Val Val Leu Gly Ala Asn Gly Thr | |
| 205 210 215 | |
| TAC AGC TGC CTG GTG CGC AAC CCC GTG CTG CAG CAG GAT GCG CAC AGC | 723 |
| Tyr Ser Cys Leu Val Arg Asn Pro Val Leu Gln Gln Asp Ala His Ser | |
| 220 225 230 | |
| TCT GTC ACC ATC ACA CCC CAG AGA AGC CCC ACA GGA GCC GTG GAG GTC | 771 |
| Ser Val Thr Ile Thr Pro Gln Arg Ser Pro Thr Gly Ala Val Glu Val | |
| 235 240 245 | |
| CAG GTC CCT GAG GAC CCG GTG GTG GCC CTA GTG GGC ACC GAT GCC ACC | 819 |
| Gln Val Pro Glu Asp Pro Val Val Ala Leu Val Gly Thr Asp Ala Thr | |
| 250 255 260 265 | |
| CTG CGC TGC TCC TTC TCC CCC GAG CCT GGC TTC AGC CTG GCA CAG CTC | 867 |
| Leu Arg Cys Ser Phe Ser Pro Glu Pro Gly Phe Ser Leu Ala Gln Leu | |
| 270 275 280 | |
| AAC CTC ATC TGG CAG CTG ACA GAC ACC AAA CAG CTG GTG CAC AGT TTC | 915 |
| Asn Leu Ile Trp Gln Leu Thr Asp Thr Lys Gln Leu Val His Ser Phe | |
| 285 290 295 | |
| ACC GAA GGC CGG GAC CAG GGC AGC GCC TAT GCC AAC CGC ACG GCC CTC | 963 |
| Thr Glu Gly Arg Asp Gln Gly Ser Ala Tyr Ala Asn Arg Thr Ala Leu | |
| 300 305 310 | |
| TTC CCG GAC CTG CTG GCA CAA GGC AAT GCA TCC CTG AGG CTG CAG CGC | 1011 |
| Phe Pro Asp Leu Leu Ala Gln Gly Asn Ala Ser Leu Arg Leu Gln Arg | |
| 315 320 325 | |
| GTG CGT GTG GCG GAC GAG GGC AGC TTC ACC TGC TTC GTG AGC ATC CGG | 1059 |
| Val Arg Val Ala Asp Glu Gly Ser Phe Thr Cys Phe Val Ser Ile Arg | |
| 330 335 340 345 | |
| GAT TTC GGC AGC GCT GCC GTC AGC CTG CAG GTG GCC GCT CCC TAC TCG | 1107 |
| Asp Phe Gly Ser Ala Ala Val Ser Leu Gln Val Ala Ala Pro Tyr Ser | |
| 350 355 360 | |
| AAG CCC AGC ATG ACC CTG GAG CCC AAC AAG GAC CTG CGG CCA GGG GAC | 1155 |
| Lys Pro Ser Met Thr Leu Glu Pro Asn Lys Asp Leu Arg Pro Gly Asp | |
| 365 370 375 | |
| ACG GTG ACC ATC ACG TGC TCC AGC TAC CGG GGC TAC CCT GAG GCT GAG | 1203 |
| Thr Val Thr Ile Thr Cys Ser Ser Tyr Arg Gly Tyr Pro Glu Ala Glu | |
| 380 385 390 | |
| GTG TTC TGG CAG GAT GGG CAG GGT GTG CCC CTG ACT GGC AAC GTG ACC | 1251 |
| Val Phe Trp Gln Asp Gly Gln Gly Val Pro Leu Thr Gly Asn Val Thr | |
| 395 400 405 | |
| ACG TCG CAG ATG GCC AAC GAG CAG GGC TTG TTT GAT GTG CAC AGC GTC | 1299 |
| Thr Ser Gln Met Ala Asn Glu Gln Gly Leu Phe Asp Val His Ser Val | |
| 410 415 420 425 | |
| CTG CGG GTG GTG CTG GGT GCG AAT GGC ACC TAC AGC TGC CTG GTG CGC | 1347 |
| Leu Arg Val Val Leu Gly Ala Asn Gly Thr Tyr Ser Cys Leu Val Arg | |
| 430 435 440 | |
| AAC CCC GTG CTG CAG CAG GAT GCG CAC GGC TCT GTC ACC ATC ACA GGG | 1395 |
| Asn Pro Val Leu Gln Gln Asp Ala His Gly Ser Val Thr Ile Thr Gly | |
| 445 450 455 | |
| CAG CCT ATG ACA TTC CCC CCA GAG GCC CTG TGG GTG ACC GTG GGG CTG | 1443 |
| Gln Pro Met Thr Phe Pro Pro Glu Ala Leu Trp Val Thr Val Gly Leu | |
| 460 465 470 | |
| TCT GTC TGT CTC ATT GCA CTG CTG GTG GCC CTG GCT TTC GTG TGC TGG | 1491 |
| Ser Val Cys Leu Ile Ala Leu Leu Val Ala Leu Ala Phe Val Cys Trp | |
| 475 480 485 | |
| AGA AAG ATC AAA CAG AGC TGT GAG GAG GAG AAT GCA GGA GCT GAG GAC | 1539 |
| Arg Lys Ile Lys Gln Ser Cys Glu Glu Glu Asn Ala Gly Ala Glu Asp | |
| 490 495 500 505 | |
| CAG GAT GGG GAG GGA GAA GGC TCC AAG ACA GCC CTG GAG CCT CTG AAA | 1587 |
| Gln Asp Gly Glu Gly Glu Gly Ser Lys Thr Ala Leu Gln Pro Leu Lys | |
| 510 515 520 | |
| CAC TCT GAC AGC AAA GAA GAT GAT GGA CAA GAA ATA GCC TGA | 1629 |
| His Ser Asp Ser Lys Glu Asp Asp Gly Gln Glu Ile Ala | |
| 525 530 534 | |
| CCATGAGGAC CAGGGAGCTG CTACCCCTCC CTACAGCTCC TACCCTCTGG CTGC | 1683 |

In another embodiment, the invention provides novel utility for isolated nucleic acid molecules comprising DNA encoding LP276 polypeptides. In another aspect, the invention provides novel utility for isolated nucleic acid molecules comprising DNA that encodes LP276 having amino acid residues from 1 or about 29 to about 534, inclusive, of SEQ ID NO:2, or that are complementary to such encoding nucleic acid sequences, and remain stably bound to them under at least moderate, and optionally, high stringency conditions. Specifically, polypeptides used in the present invention comprise the amino acid sequence as shown in SEQ ID NO:2, as well as fragments, variants, and derivatives thereof.

TABLE 2
SEQ ID NO:2, LP276 polypeptide.

```
Met Leu Arg Arg Arg Gly Ser Pro Gly Met Gly Val His Val Gly Ala
    1 5 10 15
Ala Leu Gly Ala Leu Trp Phe Cys Leu Thr Gly Ala Leu Glu Val Gln
                          20 25 30
Val Pro Glu Asp Pro Val Val Ala Leu Val Gly Thr Asp Ala Thr Leu
                  35 40 45
Cys Cys Ser Phe Ser Pro Glu Pro Gly Phe Ser Leu Ala Gln Leu Asn
          50 55 60
Leu Ile Trp Gln Leu Thr Asp Thr Lys Gln Leu Val His Ser Phe Ala
  65 70 75 80
Glu Gly Gln Asp Gln Gly Ser Ala Tyr Ala Asn Arg Thr Ala Leu Phe
                                  85 90 95
Pro Asp Leu Leu Ala Gln Gly Asn Ala Ser Leu Arg Leu Gln Arg Val
                        100 105 110
Arg Val Ala Asp Glu Gly Ser Phe Thr Cys Phe Val Ser Ile Arg Asp
                115 120 125
Phe Gly Ser Ala Ala Val Ser Leu Gln Val Ala Ala Pro Tyr Ser Lys
        130 135 140
Pro Ser Met Thr Leu Glu Pro Asn Lys Asp Leu Arg Pro Gly Asp Thr
145 150 155 160
Val Thr Ile Thr Cys Ser Ser Tyr Gln Gly Tyr Pro Glu Ala Glu Val
                                  165 170 175
Phe Trp Gln Asp Gly Gln Gly Val Pro Leu Thr Gly Asn Val Thr Thr
                        180 185 190
Ser Gln Met Ala Asn Glu Gln Gly Leu Phe Asp Val His Ser Ile Leu
                195 200 205
Arg Val Val Leu Gly Ala Asn Gly Thr Tyr Ser Cys Leu Val Arg Asn
        210 215 220
Pro Val Leu Gln Gln Asp Ala His Ser Ser Val Thr Ile Thr Pro Gln
225 230 235 240
Arg Ser Pro Thr Gly Ala Val Glu Val Gln Val Pro Glu Asp Pro Val
                                245 250 255
Val Ala Leu Val Gly Thr Asp Ala Thr Leu Arg Cys Ser Phe Ser Pro
                        260 265 270
Glu Pro Gly Phe Ser Leu Ala Gln Leu Asn Leu Ile Trp Gln Leu Thr
                275 280 285
Asp Thr Lys Gln Leu Val His Ser Phe Thr Glu Gly Arg Asp Gln Gly
        290 295 300
Ser Ala Tyr Ala Asn Arg Thr Ala Leu Phe Pro Asp Leu Leu Ala Gln
305 310 315 320
Gly Asn Ala Ser Leu Arg Leu Gln Arg Val Arg Val Ala Asp Glu Gly
                                325 330 335
Ser Phe Thr Cys Phe Val Ser Ile Arg Asp Phe Gly Ser Ala Ala Val
                        340 345 350
Ser Leu Gln Val Ala Ala Pro Tyr Ser Lys Pro Ser Met Thr Leu Glu
                355 360 365
Pro Asn Lys Asp Leu Arg Pro Gly Asp Thr Val Thr Ile Thr Cys Ser
        370 375 380
Ser Tyr Arg Gly Tyr Pro Glu Ala Glu Val Phe Trp Gln Asp Gly Gln
385 390 395 400
Gly Val Pro Leu Thr Gly Asn Val Thr Thr Ser Gln Met Ala Asn Glu
                                405 410 415
Gln Gly Leu Phe Asp Val His Ser Val Leu Arg Val Val Leu Gly Ala
                        420 425 430
Asn Gly Thr Tyr Ser Cys Leu Val Ary Asn Pro Val Leu Gln Gln Asp
                435 440 445
Ala His Gly Ser Val Thr Ile Thr Gly Gln Pro Met Thr Phe Pro Pro
        450 455 460
Glu Ala Leu Trp Val Thr Val Gly Leu Ser Val Cys Leu Ile Ala Leu
465 470 475 480
Leu Val Ala Leu Ala Phe Val Cys Trp Arg Lys Ile Lys Gln Ser Cys
                                485 490 495
Glu Glu Glu Asn Ala Gly Ala Glu Asp Gln Asp Gly Glu Gly Glu Gly
                        500 505 510
Ser Lys Thr Ala Leu Gln Pro Leu Lys His Ser Asp Ser Lys Glu Asp
                515 520 525
Asp Gly Gln Glu Ile Ala
        530 534
```

LP276 polypeptide is a transmembrane protein containing four Ig-like domains in the extracellular domain. The extracellular domain of LP276 polynucleotide comprises nucleotides from about 109 through about 1419, inclusive, of SEQ ID NO:1. The four Ig-like domains of the extracellular domain comprise nucleotides from about 151 through about 396, inclusive, of SEQ ID NO:1 (Ig-like domain 1); nucleotides from about 496 through about 690, inclusive, of SEQ ID NO:1 (Ig-like domain 2); nucleotides from about 805 through about 1050, inclusive, of SEQ ID NO:1 (Ig-like domain 3); and nucleotides from about 1150 through about 1344, inclusive, of SEQ ID NO:1 (Ig-like domain 4).

Correspondingly, the extracellular domain of LP276 polypeptide comprises amino acid residues of about 29 to about 465, inclusive, of SEQ ID NO:2. The Ig-like domains of the extracellular domain comprise amino acids from about 43 through about 124, inclusive, of SEQ ID NO:2 (Ig-like domain 1); amino acids from about 158 through about 222, inclusive, of SEQ ID NO:2 (Ig-like domain 2); amino acids from about 261 through about 342, inclusive, of SEQ ID NO:2 (Ig-like domain 3); and amino acids from about 376 through about 440, inclusive, of SEQ ID NO:2 (Ig-like domain 4).

Also contemplated by the present invention is LP276L, an active fragment of the native LP276 polypeptide, comprising the amino acid sequence encoded by nucleotides from 25 or about 109 through about 1419, inclusive, of SEQ ID NO:1. In another embodiment, the invention provides novel isolated nucleic acid molecules comprising DNA encoding the LP276L polypeptide. In another aspect, the invention provides novel isolated nucleic acids comprising DNA that encodes the LP276L polypeptide having amino acid residues from about 1 or about 29 to about 465, inclusive, of SEQ ID NO:2, or that are complementary to such encoding nucleic acid sequences, and remain stably bound to them under at least moderate, and optionally, high stringency conditions.

In another embodiment, fusion proteins of LP276 polypeptide are contemplated. In a preferred embodiment, fusion proteins of LP276L polypeptide are contemplated comprising the amino acid sequence encoded by nucleotides from 25 or about 109 through about 1419, inclusive, of SEQ ID NO:1 fused to another polypeptide. In another embodiment, the invention provides novel isolated nucleic acid molecules encoding an LP276L fusion protein comprising DNA encoding the LP276L polypeptide fused to a heterologous polypeptide. In yet another embodiment, the invention provides novel isolated nucleic acids encoding an LP276L fusion protein comprising DNA that encodes the LP276L polypeptide having amino acid residues from about 1 or about 29 to about 465, inclusive, of SEQ ID NO:2 fused to a heterologous polypeptide.

In a most preferred embodiment, Fc fusion proteins of LP276L polypeptide, herein designated as LP276ATFV, are contemplated comprising the amino acid sequence encoded by nucleotides from 1 through about 2103 or about 2151, inclusive, of SEQ ID NO:3.

TABLE 3
SEQ ID NO:3, LP276ATFV Fusion Polynucleotide.

| | |
|---|---|
| atg ctg cgt cgg cgg ggc agc cct ggc atg ggt gtg cat gtg ggt gca | 48 |
| Met Leu Arg Arg Arg Gly Ser Pro Gly Met Gly Val His Val Gly Ala | |
| 5 10 15 | |
| gcc ctg gga gca ctg tgg ttc tgc ctc aca gga gcc ctg gag gtc cag | 96 |
| Ala Leu Gly Ala Leu Trp Phe Cys Leu Thr Gly Ala Leu Glu Val Gln | |
| 20 25 30 | |
| gtc cct gaa gac cca gtg gtg gca ctg gtg ggc acc gat gcc acc ctg | 144 |
| Val Pro Glu Asp Pro Val Val Ala Leu Val Gly Thr Asp Ala Thr Leu | |
| 35 40 45 | |
| tgc tgc tcc ttc tcc cct gag cct ggc ttc agc ctg gca cag ctc aac | 192 |
| Cys Cys Ser Phe Ser Pro Glu Pro Gly Phe Ser Leu Ala Gln Leu Asn | |
| 50 55 60 | |
| ctc atc tgg cag ctg aca gat acc aaa cag ctg gtg cac agc ttt gct | 240 |
| Leu Ile Trp Gln Leu Thr Asp Thr Lys Gln Leu Val His Ser Phe Ala | |
| 65 70 75 80 | |
| gag ggc cag gac cag ggc agc gcc tat gcc aac cgc acg gcc ctc ttc | 288 |
| Glu Gly Gln Asp Gln Gly Ser Ala Tyr Ala Asn Arg Thr Ala Leu Phe | |
| 85 90 95 | |
| ccg gac ctg ctg gca cag ggc aac gca tcc ctg agg ctg cag cgc gtg | 336 |
| Pro Asp Leu Leu Ala Gln Gly Asn Ala Ser Leu Arg Leu Gln Arg Val | |
| 100 105 110 | |
| cgt gtg gcg gac gag ggc agc ttc acc tgc ttc gtg agc atc cgg gat | 384 |
| Arg Val Ala Asp Glu Gly Ser Phe Thr Cys Phe Val Ser Ile Arg Asp | |
| 115 120 125 | |
| ttc ggc agc gct gcc gtc agc ctg cag gtg gcc gct ccc tac tcg aag | 432 |
| Phe Gly Ser Ala Ala Val Ser Leu Gln Val Ala Ala Pro Tyr Ser Lys | |
| 130 135 140 | |
| ccc agc atg acc ctg gag ccc aac aag gac ctg cgg cca ggg gac acg | 480 |
| Pro Ser Met Thr Leu Glu Pro Asn Lys Asp Leu Arg Pro Gly Asp Thr | |
| 145 150 155 160 | |
| gtg acc atc acg tgc tcc agc tac cag ggc tac cct gag gct gag gtg | 528 |
| Val Thr Ile Thr Cys Ser Ser Tyr Gln Gly Tyr Pro Glu Ala Glu Val | |
| 165 170 175 | |
| ttc tgg cag gat ggg cag ggt gtg ccc ctg act ggc aac gtg acc acg | 576 |
| Phe Trp Gln Asp Gly Gln Gly Val Pro Leu Thr Gly Asn Val Thr Thr | |
| 180 185 190 | |
| tcg cag atg gcc aac gag cag ggc ttg ttt gat gtg cac agc atc ctg | 624 |
| Ser Gln Met Ala Asn Glu Gln Gly Leu Phe Asp Val His Ser Ile Leu | |
| 195 200 205 | |
| cgg gtg gtg ctg ggt gca aat ggc acc tac agc tgc ctg gtg cgc aac | 672 |
| Arg Val Val Leu Gly Ala Asn Gly Thr Tyr Ser Cys Leu Val Arg Asn | |
| 210 215 220 | |
| ccc gtg ctg cag cag gat gcg cac agc tct gtc acc atc aca ccc cag | 720 |
| Pro Val Leu Gln Gln Asp Ala His Ser Ser Val Thr Ile Thr Pro Gln | |
| 225 230 235 240 | |
| aga agc ccc aca gga gcc gtg gag gtc cag gtc cct gag gac ccg gtg | 768 |
| Arg Ser Pro Thr Gly Ala Val Glu Val Gln Val Pro Glu Asp Pro Val | |
| 245 250 255 | |
| gtg gcc cta gtg ggc acc gat gcc acc ctg cgc tgc tcc ttc tcc ccc | 816 |
| Val Ala Leu Val Gly Thr Asp Ala Thr Leu Arg Cys Ser Phe Ser Pro | |
| 260 265 270 | |
| gag cct ggc ttc agc ctg gca cag ctc aac ctc atc tgg cag ctg aca | 864 |
| Glu Pro Gly Phe Ser Leu Ala Gln Leu Asn Leu Ile Trp Gln Leu Thr | |
| 275 280 285 | |
| gac acc aaa cag ctg gtg cac agt ttc acc gaa ggc cgg gac cag ggc | 912 |
| Asp Thr Lys Gln Leu Val His Ser Phe Thr Glu Gly Arg Asp Gln Gly | |
| 290 295 300 | |
| agc gcc tat gcc aac cgc acg gcc ctc ttc ccg gac ctg ctg gca caa | 960 |
| Ser Ala Tyr Ala Asn Arg Thr Ala Leu Phe Pro Asp Leu Leu Ala Gln | |
| 305 310 315 320 | |
| ggc aat gca tcc ctg agg ctg cag cgc gtg cgt gtg gcg gac gag ggc | 1008 |
| Gly Asn Ala Ser Leu Arg Leu Gln Arg Val Arg Val Ala Asp Glu Gly | |
| 325 330 335 | |
| agc tta acc tgc ttc gtg agc atc cgg gat ttc ggc agc gct gcc gtc | 1056 |
| Ser Phe Thr Cys Phe Val Ser Ile Arg Asp Phe Gly Ser Ala Ala Val | |
| 340 345 350 | |
| agc ctg cag gtg gcc gct ccc tac tcg aag ccc agc atg acc ctg gag | 1104 |
| Ser Leu Gln Val Ala Ala Pro Tyr Ser Lys Pro Ser Met Thr Leu Glu | |
| 355 360 365 | |
| ccc aac aag gac ctg cgg cca ggg gac acg gtg acc atc acg tgc tcc | 1152 |
| Pro Asn Lys Asp Leu Arg Pro Gly Asp Thr Val Thr Ile Thr Cys Ser | |
| 370 375 380 | |
| agc tac cgg ggc tac cct gag gct gag gtg ttc tgg cag gat ggg cag | 1200 |
| Ser Tyr Arg Gly Tyr Pro Glu Ala Glu Val Phe Trp Gln Asp Gly Gln | |
| 385 390 395 400 | |
| ggt gtg ccc ctg act ggc aac gtg acc acg tcg cag atg gcc aac gag | 1248 |
| Gly Val Pro Leu Thr Gly Asn Val Thr Thr Ser Gln Met Ala Asn Glu | |
| 405 410 415 | |
| cag gyc ttg ttt gat gtg cac agc gtc ctg cgg gtg gtg ctg ggt gcg | 1296 |
| Gln Gly Leu Phe Asp Val His Ser Val Leu Ary Val Val Leu Gly Ala | |
| 420 425 430 | |
| aat ggc acc tac agc tgc ctg gtg cgc aac ccc gtg ctg cag cag gat | 1344 |
| Asn Gly Thr Tyr Ser Cys Leu Val Arg Asn Pro Val Leu Gln Gln Asp | |
| 435 440 445 | |
| gcg cac ggc tct gcc acc atc aca ggg cag cct atg aca ttc ccc cca | 1392 |
| Ala His Gly Ser Val Thr Ile Thr Gly Gln Pro Met Thr Phe Pro Pro | |
| 450 455 460 | |
| gag gat atc gag ccc aaa tct tgt gac aaa act cac aca tgc cca ccg | 1440 |
| Glu Asp Ile Glu Pro Lys Ser Cys Asp Lys Thr His Thr Cys Pro Pro | |
| 465 470 475 480 | |
| tgc cca gca cct gag ctc ctg ggg gga ccg tca gtc ttc ctc ttc ccc | 1488 |
| Cys Pro Ala Pro Glu Leu Leu Gly Gly Pro Ser Vel Phe Leu Phe Pro | |
| 485 490 495 | |
| cca aaa ccc aag gac acc ctc atg atc tcc cgg acc cct gag gtc aca | 1536 |
| Pro Lys Pro Lys Asp Thr Leu Met Ile Ser Arg Thr Pro Glu Val Thr | |
| 500 505 510 | |
| tgc gtg gtg gtg gac gtg agc cac gaa gac cct gag gtc aag ttc aac | 1584 |
| Cys Val Val Val Asp Val Ser His Glu Asp Pro Glu Val Lys Phe Asn | |
| 515 520 525 | |
| tgg tac gtg gac ggc gtg gag gtg cat aat gcc aag aca aag ccg cgg | 1632 |
| Trp Tyr Val Asp Gly Val Glu Val His Asn Ala Lys Thr Lys Pro Arg | |
| 530 535 540 | |
| gag gag cag tac aac agc acg tac cgt gtg gtc agc gtc ctc acc gtc | 1680 |
| Glu Glu Gln Tyr Asn Ser Thr Tyr Arg Val Val Ser Val Leu Thr Val | |
| 545 550 555 560 | |
| ctg cac cag gac tgg ctg aat ggc aag gag tac aag tgc aag gtc tcc | 1728 |
| Leu His Gln Asp Trp Leu Asn Gly Lys Glu Tyr Lys Cys Lys Val Ser | |
| 565 570 575 | |
| aac aaa gcc ctc cca gcc ccc atc gag aaa acc atc tcc aaa gcc aaa | 1776 |
| Asn Lys Ala Leu Pro Ala Pro Ile Glu Lys Thr Ile Ser Lys Ala Lys | |
| 580 585 590 | |
| ggg cag ccc cga gaa cca cag gag tac acc ctg ccc cca tcc cgg gag | 1824 |
| Gly Gln Pro Arg Glu Pro Gln Glu Tyr Thr Leu Pro Pro Ser Arg Glu | |
| 595 600 605 | |
| gag atg acc aag aac cag gtc agc ctg acc tgc ctg gtc aaa ggc ttc | 1872 |
| Glu Met Thr Lys Asn Gln Val Ser Leu Thr Cys Leu Val Lys Gly Phe | |
| 610 615 620 | |
| tat ccc agc gac atc gcc gtg gag tgg gag agc aat ggg cag ccg gag | 1920 |
| Tyr Pro Ser Asp Ile Ala Val Glu Trp Glu Ser Asn Gly Gln Pro Glu | |
| 625 630 635 640 | |
| aac aac tac aag acc acg cct ccc gtg ctg gac tcc gac ggc tcc ttc | 1968 |
| Asn Asn Tyr Lys Thr Thr Pro Pro Val Leu Asp Ser Asp Gly Ser Phe | |
| 645 650 655 | |
| ttc ctc tat agc aag ctc acc gtg gac aag agc agg tgg cag cag ggg | 2016 |
| Phe Leu Tyr Ser Lys Leu Thr Val Asp Lys Ser Arg Trp Gln Gln Gly | |
| 660 665 670 | |
| aac gtc ttc tca cgc tcc gtg atg cat gag gct ctg cac aac cac tac | 2064 |
| Asn Val Phe Ser Cys Ser Val Met His Glu Ala Leu His Asn His Tyr | |
| 675 680 685 | |
| acg cag aag agc ctc tcc ctg tct ccg ggt aaa agg atc gac tac aag | 2112 |
| Thr Gln Lys Ser Leu Ser Leu Ser Pro Gly Lys Arg Ile Asp Tyr Lys | |
| 690 695 700 | |
| gat gac gac gac aag cac gtg cat cac cat cac cat cac | 2151 |
| Asp Asp Asp Asp Lys His Val His His His His His His | |
| 705 710 715 717 | |

In another most preferred embodiment, the invention provides novel isolated nucleic acid molecules comprising DNA encoding the LP276ATFV polypeptide. In yet another embodiment, the invention provides novel isolated nucleic acids comprising DNA that encodes the LP276ATFV polypeptide having amino acid residues from about 1 to about 701 or about 717, inclusive, of SEQ ID NO:4.

TABLE 4
SEQ ID NO:4, LP276ATFV Fusion Polypeptide.

```
Met Leu Arg Arg Arg Gly Ser Pro Gly Met Gly Val His Val Gly Ala
    1 5 10 15
Ala Leu Gly Ala Leu Trp Phe Cys Leu Thr Gly Ala Leu Glu Val Gln
                          20 25 30
Val Pro Glu Asp Pro Val Val Ala Leu Val Gly Thr Asp Ala Thr Leu
                  35 40 45
Cys Cys Ser Phe Ser Pro Glu Pro Gly Phe Ser Leu Ala Gln Leu Asn
          50 55 60
Leu Ile Trp Gln Leu Thr Asp Thr Lys Gln Leu Val His Ser Phe Ala
  65 70 75 80
Glu Gly Gln Asp Gln Gly Ser Ala Tyr Ala Asn Arg Thr Ala Leu Phe
                                  85 90 95
Pro Asp Leu Leu Ala Gln Gly Asn Ala Ser Leu Arg Leu Gln Arg Val
                        100 105 110
Arg Val Ala Asp Glu Gly Ser Phe Thr Cys Phe Val Ser Ile Arg Asp
                115 120 125
Phe Gly Ser Ala Ala Val Ser Leu Gln Val Ala Ala Pro Tyr Ser Lys
        130 135 140
Pro Ser Met Thr Leu Glu Pro Asn Lys Asp Leu Arg Pro Gly Asp Thr
145 150 155 160
Val Thr Ile Thr Cys Ser Ser Tyr Gln Gly Tyr Pro Glu Ala Glu Val
                                165 170 175
Phe Trp Gln Asp Gly Gln Gly Val Pro Leu Thr Gly Asn Val Thr Thr
                        180 185 190
Ser Gln Met Ala Asn Glu Gln Gly Leu Phe Asp Val His Ser Ile Leu
                195 200 205
Arg Val Val Leu Gly Ala Asn Gly Thr Tyr Ser Cys Leu Val Arg Asn
        210 215 220
Pro Val Leu Gln Gln Asp Ala His Ser Ser Val Thr Ile Thr Pro Gln
225 230 235 240
Arg Ser Pro Thr Gly Ala Val Glu Val Gln Val Pro Glu Asp Pro Val
                                245 250 255
Val Ala Leu Val Gly Thr Asp Ala Thr Leu Arg Cys Ser Phe Ser Pro
                        260 265 270
Glu Pro Gly Phe Ser Leu Ala Gln Leu Asn Leu Ile Trp Gln Leu Thr
                275 280 285
Asp Thr Lys Gln Leu Val His Ser Phe Thr Glu Gly Arg Asp Gln Gly
        290 295 300
Ser Ala Tyr Ala Asn Arg Thr Ala Leu Phe Pro Asp Leu Leu Ala Gln
305 310 315 320
Gly Asn Ala Ser Leu Arg Leu Gln Arg Val Arg Val Ala Asp Glu Gly
                                325 330 335
Ser Phe Thr Cys Phe Val Ser Ile Arg Asp Phe Gly Ser Ala Ala Val
                        340 345 350
Ser Leu Gln Val Ala Ala Pro Tyr Ser Lys Pro Ser Met Thr Leu Glu
                355 360 365
Pro Asn Lys Asp Leu Arg Pro Gly Asp Thr Val Thr Ile Thr Cys Ser
        370 375 380
Ser Tyr Arg Gly Tyr Pro Glu Ala Glu Val Phe Trp Gln Asp Gly Gln
385 390 395 400
Gly Val Pro Leu Thr Gly Asn Val Thr Thr Ser Gln Met Ala Asn Glu
                                405 410 415
Gln Gly Leu Phe Asp Val His Ser Val Leu Arg Val Val Leu Gly Ala
                        420 425 430
Asn Gly Thr Tyr Ser Cys Leu Val Arg Asn Pro Val Leu Gln Gln Asp
                435 440 445
Ala His Gly Ser Val Thr Ile Thr Gly Gln Pro Met Thr Phe Pro Pro
        450 455 460
Glu Asp Ile Glu Pro Lys Ser Cys Asp Lys Thr His Thr Cys Pro Pro
465 470 475 480
Cys Pro Ala Pro Glu Leu Leu Gly Gly Pro Ser Val Phe Leu Phe Pro
                                485 490 495
Pro Lys Pro Lys Asp Thr Leu Met Ile Ser Arg Thr Pro Glu Val Thr
                        500 505 510
Cys Val Val Val Asp Val Ser His Glu Asp Pro Glu Val Lys Phe Asn
                515 520 525
Trp Tyr Val Asp Gly Val Glu Val His Asn Ala Lys Thr Lys Pro Arg
        530 535 540
Glu Glu Gln Tyr Asn Ser Thr Tyr Arg Val Val Ser Val Leu Thr Val
545 550 555 560
Leu His Gln Asp Trp Leu Asn Gly Lys Glu Tyr Lys Cys Lys Val Ser
                                565 570 575
Asn Lys Ala Leu Pro Ala Pro Ile Glu Lys Thr Ile Ser Lys Ala Lys
                        580 585 590
Gly Gln Pro Arg Glu Pro Gln Glu Tyr Thr Leu Pro Pro Ser Arg Glu
                595 600 605
Glu Met Thr Lys Asn Gln Val Ser Leu Thr Cys Leu Val Lys Gly Phe
        610 615 620
Tyr Pro Ser Asp Ile Ala Val Glu Trp Glu Ser Asn Gly Gln Pro Glu
625 630 635 640
Asn Asn Tyr Lys Thr Thr Pro Pro Val Leu Asp Ser Asp Gly Ser Phe
                                645 650 655
Phe Leu Tyr Ser Lys Leu Thr Val Asp Lys Ser Arg Trp Gln Gln Gly
                        660 665 670
Asn Val Phe Ser Cys Ser Val Met His Glu Ala Leu His Asn His Tyr
                675 680 685
Thr Gln Lys Ser Leu Ser Leu Ser Pro Gly Lys Arg Ile Asp Tyr Lys
        690 695 700
Asp Asp Asp Asp Lys His Val His His His His His His
705 710 715 717
```

LP276 has sequence similarity to the human B7 family of proteins, a group of costimulatory molecules belonging to the immunoglobulin (Ig) superfamily. Chapoval, et al., Nature Immunol. 2(3):269-74 (2001). This family is characterized by four conserved cysteines, common to each family member. Additionally, each molecule has repeating motifs known as Ig-like domains, with most family members containing two Ig-like domains. Although LP276 contains four Ig-like domains as opposed to two, LP276 exhibits high sequence similarity to other B7 family members, as shown in Table 5.

TABLE 5
Comparison of LP276 to B7 family members.

| | |
|---|---|
| LP276 | MLRRRGSPGMGVHVGAA---LGALWFCLTGALEVQVPED--PVVALV |
| hB7-H3 | MLRRRGSPGMGVHVGAA---LGALWFCLTGALEVQVPED--PVVALV |
| hB7-H2 | MRLGS------PGLLFLLPS-SLRADTQEK--EVRAMV |
| hB7-H1 | MRIFAVFT---FMTYWHLLN-AFTVTVPKD--LYVVEY |
| hB7-2 | MGLSN--------ILFVMAF-LLSGAAPLK---IQAYF |
| hB7-1 | MGHTRRQGTSPSKCPYLNFFQLLVLAGLSHFCSGVIHVTKEV |
| LP276 | GTDATLCCSFSPEPGFSLAQLNLIWQLTD--TKQLVHSFAE--GQDQ |
| hB7-H3 | GTDATLCCSFSPEPCFSLAQLNLIWQLTD--TKQLVHSFAE--GQDQ |
| hB7-H2 | GSDVELSCACPEGSRFDLNDVYVYWQTSESKTVVTYHIPQNSSLENV |
| hB7-H1 | GSNMTIECKFPVEKQLDLAALIVYWEMEDKNIIQFVHGEED--LKVQ |
| hB7-2 | NETADLPCQPANSQNQSLSELVVFWQDQENLVLNEVYLGKEK-FDSV |
| hB7-1 | KEVATLSCGHNVS-VEELAQTRIYWQKEK--KMVLTMMSGD---MNI |
| LP276 | GSAYANRTALFPDLLAQGNASLRLQRVRVADEGSFTCFVS-IRDFGS |
| hB7-H3 | GSAYANRTALFPDLLAQGNASLRLQRVRVADEGSFTCFVS-IRDFGS |
| hB7-H2 | DSRYRNRALMSPAGMLRGDFSLRLFNVTPQDEQKFHCLVL-SQSLGF |
| hB7-H1 | HSSYRQRARLLKDQLSLGNAALQITDVKLQDAGVYRCMIS-YGGADY |
| hB7-2 | HSKYMGRTSFD-----SDSWTLRLHNLQIKDKGLYQCIIHHKKPTGM |
| hB7-1 | WPEYKNRTIFD----ITNNLSIVILALRPSDEGTYECVVLKYEKDAF |
| LP276 | AAVS-LQVAAPYSKPSMTLEPNKDLRPGD--TVTITCSSYQGYPEAE |
| hB7-H3 | AAVS-LQVAAPYSKPSMTLEPNKDLRPGD--TVTITCSSYQGYPEAE |
| hB7-H2 | QEVLSVEVTLHVAANFSVPVVSAPHSPSQD-ELTFTCTSINGYPRPN |
| hB7-H1 | KRIT-VKVNAPYN---KINQRILVVDPVTS-EHELTCQA-EGYPKAE |
| hB7-2 | IRIHQMNSELSVLANFSQPEIVPISNITENVYINLTCSSIHGYPEPK |
| hB7-1 | KREHLAEVTLSVKADFPTPSISDFEIPTSN-IRRIICSTSGGFPEPH |
| LP276 | ---VFWQDGQGVPLTGNVTTSQMANE--QGLFDVHSILRVVL---GA |
| hB7-H3 | ---VFWQDGQGVPLTGNVTTSQMANE--QGLFDVHSVLRVVL---GA |
| hB7-H2 | ---VYWINKTDNSLLDQALQNDTVFLNMRGLYDVVSVLRIAR---TP |
| hB7-H1 | ---VIWTSSDHQVLSGKTTTTNSKRE--EKLFNVTSTLRINT---TT |
| hB7-2 | KMSVLLRTKNSTIEYDGIMQKSQDNV--TELYDVSISLSVSFPDVTS |
| hB7-1 | ---LSWLE NGEELNAINTTVSQDPE--TELYAVSSKLDPNM---TT |
| LP276 | NGTYSCLVRNPVLQQ---------DAHSS-VTITPQRSPTGAVEVQV |
| hB7-H3 | NGTYSCLVRNPVLQQ---------DAHGS-VTITGQPMTFPPEALWV |
| hB7-H2 | SVNIGCCIENVLLQQNLTVGSQTGNDIGERDKITENPVSTGEKN-AA |
| hB7-H1 | NEIFYCTFRRLDPEE---------NHTAE-LVIPELPLAHPPNERTH |
| hB7-2 | NMTIFCILETDKTRLL--------SSPFS--IELEDPQPPPDHIPWI |
| hB7-1 | NHSFMCLIKYGHLRV---------NQTFN---WNTTKQEHFPDNLLP |
| LP276 | PEDPVVALVGTDATLRCSFSPEPGFSLAQLNLIWQLTDTKQLVHSFT |
| hB7-H3 | ----------------------------------------------- |
| hB7-H2 | ----------------------------------------------- |
| hB7-H1 | ----------------------------------------------- |
| hB7-2 | ----------------------------------------------- |
| hB7-1 | ----------------------------------------------- |
| LP276 | EGRDQGSAYANRTALFPDLLAQGNASLRLQRVRVADEGSFTCPVSIR |
| hB7-H3 | ----------------------------------------------- |
| hB7-H2 | ----------------------------------------------- |
| hB7-H1 | ----------------------------------------------- |
| hB7-2 | ----------------------------------------------- |
| hB7-1 | ----------------------------------------------- |
| LP276 | DFGSAAVSLQVAAPYSKPSMTLEPNKDLRPGDTVTITCSSYRGYPEA |
| hB7-H3 | ----------------------------------------------- |
| hB7-H2 | ----------------------------------------------- |
| hB7-H1 | ----------------------------------------------- |
| hB7-2 | ----------------------------------------------- |
| hB7-1 | ----------------------------------------------- |
| LP276 | EVFWQDGQGVPLTGNVTTSQMANEQGLFDVHSVLRVVLGANGTYSCL |
| hB7-H3 | ----------------------------------------------- |
| hB7-H2 | ----------------------------------------------- |
| hB7-H1 | ----------------------------------------------- |
| hB7-2 | ----------------------------------------------- |
| hB7-1 | ----------------------------------------------- |
| LP276 | VRNPVLQQDAHGSVTITGQPMTFPPEALWVTVGLSVCLIALLVALAF |
| hB7-H3 | ------------------------------TVGLSVCLIALLVALAF |
| hB7-H2 | ------------------------------TWSILAVLCLLVVVAVA |
| hB7-H1 | ------------------------------LVILGAILLCLGVALTF |
| hB7-2 | ------------------------------TAVLPTVIICVMVFCLI |
| hB7-1 | ------------------------------SWAITLISVNGIFVICC |
| LP276 | VC-WRKIKQSCEEENAGAEDQDGEGEGSKTALQPLKHSDSKEDDGQE |
| hB7-H3 | VC-WRKIKQSCEEENAGAEDQDGEGEGSKTALQPLKHSDSKEDDGQE |
| hB7-H2 | IG-WVCRDRCLQHSYAGAWAVSPET-------ELTGHV· |
| hB7-H1 | IF-RLRKGRMMDVKKCGIQDTNSKK-------QSDTHLEET· |
| hB7-2 | LWKWKKKKRPRNSYKCGTNTMEREES-EQTKKREKIHIPERSDEAQR |
| hB7-1 | LT-YCFAPRCRERR--RNERLRRES-------VRPV· |
| LP276 | IA· |
| hB7-H3 | IA· |
| hB7-H2 | |
| hB7-H1 | |
| hB7-2 | VFKSSKTSSCDKSDTCF· |
| hB7-1 | |

In contrast to family members containing two Ig-like domains, LP276 polypeptide is not costimulatory for T cell activation and IFN-gamma production. Rather, LP276 polypeptide, LP276L polypeptide, and LP276ATFV fusion protein, each containing four Ig-like domains, inhibit proliferation of T cells and IFN-gamma. Accordingly, compositions comprising LP276, LP276L, or LP276ATFV polypeptides, or polynucleotides are useful for the diagnosis, treatment, and intervention of sepsis, gram negative bacteremia, inflammation, allergic autoimmune diseases, allergic responses, infectious diseases, immunodeficiencies, type 1 diabetes, Th1-dependent insulitis, pancreatitis, aberrant apoptosis, cancers, rheumatoid arthritis, eczema, psoriasis, atopy, asthma, fibrosing lung disease, acute respiratory distress syndrome (ARDS), inflammatory bowel disease, multiple sclerosis, Hashimoto's thyroiditis, Graves' disease, systemic lupus erythematosis, vasculitis, autoimmune gastritis, HIV, HIV-induced lymphoma, fulminant viral hepatitis B, fulminant viral hepatitis C, chronic hepatitis, chronic cirrhosis, liver failure, chronic glomerulonephritis, thrombotic thrombocytopenic purpura (TTP), hemolytic uremic syndrome (HUS), aplastic anemia, myelodysplasia, transplant rejection, H. pylori associated ulceration, cytoprotection during cancer treatment, recuperation during chemotherapy, recuperation from irradiation therapy, and multiple organ dysfunction syndrome (MODS).

The present invention provides another LP276 analog, LP276S, comprising the amino acid sequence of the open reading frame encoded by the polynucleotide sequence as shown in SEQ ID NO:5. The isolated nucleic acid comprises DNA consisting of nucleotides 25 or about 109 through about 969, inclusive, of SEQ ID NO:5.

TABLE 6
SEQ ID NO:5, LP276S polynucleotide.

| | |
|---|---|
| AGCTGTCAGC CGCCTCACAG GAAG | 24 |
| ATG CTG CGT CGG CGG GGC AGC CCT GGC ATG GGT GTG CAT GTG GGT GCA | 72 |
| Met Leu Arg Arg Arg Gly Ser Pro Gly Met Gly Val His Val Gly Ala | |
| 1 5 10 15 | |
| GCC CTG GGA GCA CTG TGG TTC TGC CTC ACA GGA GCC CTG GAG GTC CAG | 120 |
| Ala Leu Gly Ala Leu Trp Phe Cys Leu Thr Gly Ala Leu Glu Val Gln | |
| 20 25 30 | |
| GTC CCT GAA GAC CCA GTG GTG GCA CTG GTG GGC ACC GAT GCC ACC CTG | 168 |
| Val Pro Glu Asp Pro Val Val Ala Leu Val Gly Thr Asp Ala Thr Leu | |
| 35 40 45 | |
| TGC TGC TCC TTC TCC CCT GAG CCT GGC TTC AGC CTG GCA CAG CTC AAC | 216 |
| Cys Cys Ser Phe Ser Pro Glu Pro Gly Phe Ser Leu Ala Gln Leu Asn | |
| 50 55 60 | |
| CTC ATC TGG CAG CTG ACA GAT ACC AAA CAG CTG GTG CAC AGC TTT GCT | 264 |
| Leu Ile Trp Gln Leu Thr Asp Thr Lys Gln Leu Val His Ser Phe Ala | |
| 65 70 75 80 | |
| GAG GGC CAG GAC CAG GGC AGC GCC TAT GCC AAC CGC ACG GCC CTC TTC | 312 |
| Glu Gly Gln Asp Gln Gly Ser Ala Tyr Ala Asn Arg Thr Ala Leu Phe | |
| 85 90 95 | |
| CCG GAC CTG CTG GCA CAG GGC AAC GCA TCC CTG AGG CTG CAG CGC GTG | 360 |
| Pro Asp Leu Leu Ala Gln Gly Asn Ala Ser Leu Arg Leu Gln Ary Val | |
| 100 105 110 | |
| CGT GTG GCG GAC GAG GGC AGC TTC ACC TGC TTC GTG AGC ATC CGG GAT | 408 |
| Arg Val Ala Asp Glu Gly Ser Phe Thr Cys Phe Val Ser Ile Arg Asp | |
| 115 120 125 | |
| TTC GGC AGC GCT GCC GTC AGC CTG CAG GTG GCC GCT CCC TAC TCG AAG | 456 |
| Phe Gly Ser Ala Ala Val Ser Leu Gln Val Ala Ala Pro Tyr Ser Lys | |
| 130 135 140 | |
| CCC AGC ATG ACC CTG GAG CCC AAC AAG GAC CTG CGG CCA GGG GAC ACG | 504 |
| Pro Ser Met Thr Leu Glu Pro Asn Lys Asp Leu Arg Pro Gly Asp Thr | |
| 145 150 155 160 | |
| GTG ACC ATC ACG TGC TCC AGC TAC CAG GGC TAC CCT GAG GCT GAG GTG | 552 |
| Val Thr Ile Thr Cys Ser Ser Tyr Gln Gly Tyr Pro Glu Ala Glu Val | |
| 165 170 175 | |
| TTC TGG CAG GAT GGG CAG GGT GTG CCC CTG ACT GGC AAC GTG ACC ACG | 600 |
| Phe Trp Gln Asp Gly Gln Gly Val Pro Leu Thr Gly Asn Val Thr Thr | |
| 180 185 190 | |
| TCG CAG ATG GCC AAC GAG CAG GGC TTG TTT GAT GTG CAC AGC ATC CTG | 648 |
| Ser Gln Met Ala Asn Glu Gln Gly Leu Phe Asp Val His Ser Ile Leu | |
| 195 200 205 | |
| CGG GTG GTG CTG GGT GCA AAT GGC ACC TAC AGC TGC CTG GTG CGC AAC | 696 |
| Arg Val Val Leu Gly Ala Asn Gly Thr Tyr Ser Cys Leu Val Arg Asn | |
| 210 215 220 | |
| CCC GTG CTG CAG CAG GAT GCG CAC AGC TCT GTC ACC ATC ACA CCC CAG | 744 |
| Pro Val Leu Gln Gln Asp Ala His Ser Ser Val Thr Ile Thr Pro Gln | |
| 225 230 235 240 | |
| AGA AGC CCC ACA GGA GCC GTG GAG GTC CAG GTC GTG GGG CTG TCT GTC | 792 |
| Arg Ser Pro Thr Gly Ala Val Glu Val Gln Val Val Gly Leu Ser Val | |
| 245 250 255 | |
| TGT CTC ATT GCA CTG CTG GTG GCC CTG GCT TTC GTG TGC TGG AGA AAG | 840 |
| Cys Leu Ile Ala Leu Leu Val Ala Leu Ala Phe Val Cys Trp Arg Lys | |
| 260 265 270 | |
| ATC AAA CAG AGC TGT GAG GAG GAG AAT GCA GGA GCT GAG GAC CAG GAT | 888 |
| Ile Lys Gln Ser Cys Glu Glu Glu Asn Ala Gly Ala Glu Asp Gln Asp | |
| 275 280 285 | |
| GGG GAG GGA GAA GGC TCC AAG ACA GCC CTG CAG CCT CTG AAA CAC TCT | 936 |
| Gly Glu Gly Glu Gly Ser Lys Thr Ala Leu Gln Pro Leu Lys His Ser | |
| 290 295 300 | |
| GAC AGC AAA GAA GAT GAT GGA CAA GAA ATA GCC TGA | 972 |
| Asp Ser Lys Glu Asp Asp Gly Gln Glu Ile Ala | |
| 305 310 315 | |
| CCATGAGGAC CAGGGAGCTG CTACCCCTCC CTACAGCTCC TACCCTCTGG CTGC | 1026 |

In one embodiment, the invention provides isolated nucleic acid molecules comprising DNA encoding LP276S polypeptides. In another embodiment, the invention provides isolated nucleic acid molecules comprising DNA that encodes LP276S having amino acid residues from 1 or about 29 to about 315, inclusive, of SEQ ID NO:6, or that are complementary to such encoding nucleic acid sequences, and remain stably bound to them under at least moderate, and optionally, high stringency conditions. Specifically, polypeptides used in the present invention comprise the amino acid sequence as shown in SEQ ID NO:6, as well as fragments, variants, and derivatives thereof.

TABLE 7
SEQ ID NO:6, LP276S polypeptide.

```
Met Leu Arg Arg Arg Gly Ser Pro Gly Met Gly Val His Val Gly Ala
    1 5 10 15
Ala Leu Gly Ala Leu Trp Phe Cys Leu Thr Gly Ala Leu Glu Val Gln
                          20 25 30
Val Pro Glu Asp Pro Val Val Ala Leu Val Gly Thr Asp Ala Thr Leu
                  35 40 45
Cys Cys Ser Phe Ser Pro Glu Pro Gly Phe Ser Leu Ala Gln Leu Asn
          50 55 60
Leu Ile Trp Gln Leu Thr Asp Thr Lys Gln Leu Val His Ser Phe Ala
  65 70 75 80
Glu Gly Gln Asp Gln Gly Ser Ala Tyr Ala Asn Arg Thr Ala Leu Phe
                                  85 90 95
Pro Asp Leu Leu Ala Gln Gly Asn Ala Ser Leu Arg Leu Gln Arg Val
                                100 105 110
Arg Val Ala Asp Glu Gly Ser Phe Thr Cys Phe Val Ser Ile Arg Asp
                115 120 125
Phe Gly Ser Ala Ala Val Ser Leu Gln Val Ala Ala Pro Tyr Ser Lys
        130 135 140
Pro Ser Met Thr Leu Glu Pro Asn Lys Asp Leu Arg Pro Gly Asp Thr
145 150 155 160
Val Thr Ile Thr Cys Ser Ser Tyr Gln Gly Tyr Pro Glu Ala Glu Val
                                165 170 175
Phe Trp Gln Asp Gly Gln Gly Val Pro Leu Thr Gly Asn Val Thr Thr
                        180 185 190
Ser Gln Met Ala Asn Glu Gln Gly Leu Phe Asp Val His Ser Ile Leu
                195 200 205
Arg Val Val Leu Gly Ala Asn Gly Thr Tyr Ser Cys Leu Val Arg Asn
        210 215 220
Pro Val Leu Gln Gln Asp Ala His Ser Ser Val Thr Ile Thr Pro Gln
225 230 235 240
Arg Ser Pro Thr Gly Ala Val Glu Val Gln Val Val Gly Leu Ser Val
                                245 250 255
Cys Leu Ile Ala Leu Leu Val Ala Leu Ala Phe Val Cys Trp Arg Lys
                        260 265 270
Ile Lys Gln Ser Cys Glu Glu Glu Asn Ala Gly Ala Glu Asp Gln Asp
                275 280 285
Gly Glu Gly Glu Gly Ser Lys Thr Ala Leu Gln Pro Leu Lys His Ser
        290 295 300
Asp Ser Lys Glu Asp Asp Gly Gln Glu Ile Ala
305 310 315
```

In another preferred embodiment, fusion proteins of LP276S polypeptide are contemplated comprising the amino acid sequence encoded by nucleotides from 25 through about 969, inclusive, of SEQ ID NO:5 fused to another polypeptide. In another embodiment, the invention provides novel isolated nucleic acid molecules encoding an LP276S fusion protein comprising DNA encoding the LP276S polypeptide fused to another polypeptide. In yet another embodiment, the invention provides novel isolated nucleic acids encoding an LP276S fusion protein comprising DNA that encodes the LP276S polypeptide having amino acid residues from about 1 to about 315, inclusive, of SEQ ID NO:6 fused to another polypeptide.

In a most preferred embodiment, Fc fusion proteins of LP276S polypeptide, herein designated as LP276ATFV2, are contemplated comprising the amino acid sequence encoded by nucleotides from 1 through about 1469 or about 1509, inclusive, of SEQ ID NO:7.

TABLE 8
SEQ ID NO:7, LP276ATFV2 Fusion Polynucleotide.

| | |
|---|---|
| atg ctg cgt cgg cgg ggc agc cct ggc atg ggt gtg cat gtg ggt gca | 48 |
| Met Leu Arg Arg Arg Gly Ser Pro Gly Met Gly Val His Val Gly Ala | |
| 1 5 10 15 | |
| gcc ctg gga gca ctg tgg ttc tgc ctc aca gga gcc ctg gag gtc cag | 96 |
| Ala Leu Gly Ala Leu Trp Phe Cys Leu Thr Gly Ala Leu Glu Val Gln | |
| 20 25 30 | |
| gtc cct gaa gac cca gtg gtg gca ctg gtg ggc acc gat gcc acc ctg | 144 |
| Val Pro Glu Asp Pro Val Val Ala Leu Val Gly Thr Asp Ala Thr Leu | |
| 35 40 45 | |
| tgc tgc tcc ttc tcc cct gag cct ggc ttc agc ctg gca cag ctc aac | 192 |
| Cys Cys Ser Phe Ser Pro Glu Pro Gly Phe Ser Leu Ala Gln Leu Asn | |
| 50 55 60 | |
| ctc atc tgg cag ctg aca gat acc aaa cag ctg gtg cac agc ttt gct | 240 |
| Leu Ile Trp Gln Leu Thr Asp Thr Lys Gln Leu Val His Ser Phe Ala | |
| 65 70 75 80 | |
| gag ggc cag gac cag ggc agc gcc tat gcc aac cgc acg gcc ctc ttc | 288 |
| Glu Gly Gln Asp Gln Gly Ser Ala Tyr Ala Asn Arg Thr Ala Leu Phe | |
| 85 90 95 | |
| ccg gac ctg ctg gca cag ggc aac gca tcc ctg agg ctg cag cgc gtg | 336 |
| Pro Asp Leu Leu Ala Gln Gly Asn Ala Ser Leu Arg Leu Gln Arg Val | |
| 100 105 110 | |
| cgt gtg gcg gac gag ggc agc ttc acc tgc ttc gtg agc atc cgg gat | 384 |
| Arg Val Ala Asp Gln Gly Ser Phe Thr Cys Phe Vel Ser Ile Arg Asp | |
| 115 120 125 | |
| ttc ggc agc gct gcc gtc agc ctg cag gtg gcc gct ccc tac tcg aag | 432 |
| Phe Gly Ser Ala Ala Val Ser Leu Gln Val Ala Ala Pro Tyr Ser Lys | |
| 130 135 140 | |
| ccc agc atg acc ctg gag ccc aac aag gac ctg cgg cca ggg gac acg | 480 |
| Pro Ser Met Thr Leu Glu Pro Asn Lys Asp Leu Arg Pro Gly Asp Thr | |
| 145 150 155 160 | |
| gtg acc atc acg tgc tcc agc tac cag ggc tac cct gag gct gag gtg | 528 |
| Val Thr Ile Thr Cys Ser Ser Tyr Gln Gly Tyr Pro Glu Ala Glu Val | |
| 165 170 175 | |
| ttc tgg cag gat ggg cag ggt gtg ccc ctg act ggc aac gtg acc acg | 576 |
| Phe Trp Gln Asp Gly Gln Gly Val Pro Leu Thr Gly Asn Val Thr Thr | |
| 180 185 190 | |
| tcg cag atg gcc aac gag cag ggc ttg ttt gat gtg cac agc atc ctg | 624 |
| Ser Gln Met Ala Asn Glu Gln Gly Leu Phe Asp Val His Ser Ile Leu | |
| 195 200 205 | |
| cgg gtg gtg ctg ggt gca aat ggc acc tac agc tgc ctg gtg cgc aac | 672 |
| Arg Val Val Leu Gly Ala Asn Gly Thr Tyr Ser Cys Leu Val Arg Asn | |
| 210 215 220 | |
| ccc gtg ctg cag cag gat gcg cac agc tct gtc acc atc aca ccc cag | 720 |
| Pro Val Leu Gln Gln Asp Ala His Ser Ser Val Thr Ile Thr Pro Gln | |
| 225 230 235 240 | |
| aga agc ccc aca gga gcc gtg gag gtc cag gtc gat atc gag ccc aaa | 768 |
| Arg Ser Pro Thr Gly Ala Val Glu Val Gln Val Asp Ile Glu Pro Lys | |
| 245 250 255 | |
| tct tgt gac aaa act cac aca tgc cca ccg tgc cca gca cct gag ctc | 816 |
| Ser Cys Asp Lys Thr His Thr Cys Pro Pro Cys Pro Ala Pro Glu Leu | |
| 260 265 270 | |
| ctg ggg gga ccg tca gtc ttc ctc ttc ccc caa aaa ccc aag gac acc | 864 |
| Leu Gly Gly Pro Ser Val Phe Leu Phe Pro Pro Lys Pro Lys Asp Thr | |
| 275 280 285 | |
| ctc atg atc tcc cgg acc cct gag gtc aca tgc gtg gtg gtg gac gtg | 912 |
| Leu Met Ile Ser Arg Thr Pro Glu Val Thr Cys Val Val Val Asp Val | |
| 290 295 300 | |
| agc cac gaa gac cct gag gtc aag ttc aac tgg tac gtg gac ggc gtg | 960 |
| Ser His Glu Asp Pro Glu Val Lys Phe Asn Trp Tyr Val Asp Gly Val | |
| 305 310 315 320 | |
| gag gtg cat aat gcc aag aca aag ccg cgg gag gag cag tac aac agc | 1008 |
| Glu Val His Asn Ala Lys Thr Lys Pro Arg Glu Glu Gln Tyr Asn Ser | |
| 325 330 335 | |
| acg tac cgt gtg gtc agc gtc ctc acc gtc ctg cac cag gac tgg ctg | 1056 |
| Thr Tyr Arg Val Val Ser Val Leu Thr Val Leu His Gln Asp Trp Leu | |
| 340 345 350 | |
| aat ggc aag gag tac aag tgc aag gtc tcc aac aaa gcc ctc cca gcc | 1104 |
| Asn Gly Lys Glu Tyr Lys Cys Lys Val Ser Asn Lys Ala Leu Pro Ala | |
| 355 360 365 | |
| ccc atc gag aaa acc atc tcc aaa gcc aaa ggg cag ccc cga gaa cca | 1152 |
| Pro Ile Glu Lys Thr Ile Ser Lys Ala Lys Gly Gln Pro Arg Glu Pro | |
| 370 375 380 | |
| cag gag tac acc atg ccc cca tcc cgg gag gag atg acc aag aac cag | 1200 |
| Gln Glu Tyr Thr Leu Pro Pro Ser Arg Gln Glu Met Thr Lys Asn Gln | |
| 385 390 395 400 | |
| gtc agc ctg acc tgc ctg gtc aaa ggc ttc tat ccc agc gac atc gcc | 1248 |
| Val Ser Leu Thr Cys Leu Val Lys Gly Phe Tyr Pro Ser Asp Ile Ala | |
| 405 410 425 | |
| gtg gag tgg gag agc aat ggg cag ccg gag aac aac tac cct aag acc | 1296 |
| Val Glu Trp Glu Ser Asn Gly Gln Pro Gln Asn Asn Tyr Lys Thr Thr | |
| 420 425 430 | |
| acg ccc gtg ctg gac tcc gac ggc tcc ttc ttc ctc tat agc aag ctc | 1344 |
| Pro Pro Val Leu Asp Ser Asp Gly Ser Phe Phe Leu Tyr Ser Lys Leu | |
| 435 440 445 | |
| acc gtg gac aag agc agg tgg cag cag ggg aac gtc ttc tca tgc tcc | i392 |
| Thr Val Asp Lys Ser Arg Trp Gln Gln Gly Asn Val Phe Ser Cys Ser | |
| 450 455 480 | |
| gtg atg cat gag gct ctg cac aac cac tac acg cag aag agc ctc tcc | 1440 |
| Val Met His Glu Ala Leu His Asn His Tyr Thr Gln Lys Ser Leu Ser | |
| 465 470 475 480 | |
| ctg tct ccg ggt aaa agg atc gac tac aag gat gac gac gac aag cac | 1488 |
| Leu Ser Pro Gly Lys Arg Ile Asp Tyr Lys Asp Asp Asp Asp Lys His | |
| 485 490 495 | |
| gtg cat cac cat cac cat cac | 1509 |
| Val His His His His His His | |
| 500 503 | |

In another most preferred embodiment, the invention provides novel isolated nucleic acid molecules comprising DNA encoding the LP276ATFV2 polypeptide. In yet another embodiment, the invention provides novel isolated nucleic acids comprising DNA that encodes the LP276ATFV2 polypeptide having amino acid residues from about 1 to about 487 or about 503, inclusive, of SEQ ID NO:8.

TABLE 9
SEQ ID NO:8, LP276ATFV2 Fusion Polypeptide.

```
Met Leu Arg Arg Arg Gly Ser Pro Gly Met Gly Val His Val Gly Ala
    1 5 10 15
Ala Leu Gly Ala Leu Trp Phe Cys Leu Thr Gly Ala Leu Glu Val Gln
                          20 25 30
Val Pro Glu Asp Pro Val Val Ala Leu Val Gly Thr Asp Ala Thr Leu
                  35 40 45
Cys Cys Ser Phe Ser Pro Glu Pro Gly Phe Ser Leu Ala Gln Leu Asn
          50 55 60
Leu Ile Trp Gln Leu Thr Asp Thr Lys Gln Leu Val His Ser Phe Ala
  65 70 75 80
Glu Gly Gln Asp Gln Gly Ser Ala Tyr Ala Asn Arg Thr Ala Leu Phe
                                  85 90 95
Pro Asp Leu Leu Ala Gln Gly Asn Ala Ser Leu Arg Leu Gln Arg Val
                        100 105 110
Arg Val Ala Asp Glu Gly Ser Phe Thr Cys Phe Val Ser Ile Arg Asp
                115 120 125
Phe Gly Ser Ala Ala Val Ser Leu Gln Val Ala Ala Pro Tyr Ser Lys
        130 135 140
Pro Ser Met Thr Leu Glu Pro Asn Lys Asp Leu Arg Pro Gly Asp Thr
145 150 155 160
Val Thr Ile Thr Cys Ser Ser Tyr Gln Gly Tyr Pro Glu Ala Glu Val
                                165 170 175
Phe Trp Gln Asp Gly Gln Gly Val Pro Lau Thr Gly Asn Val Thr Thr
                        180 185 190
Ser Gln Met Ala Asn Glu Gln Gly Leu Phe Asp Val His Ser Ile Leu
                195 200 205
Arg Val Val Leu Gly Ala Asn Gly Thr Tyr Ser Cys Leu Val Arg Asn
        210 215 220
Pro Val Leu Gln Gln Asp Ala His Ser Ser Val Thr Ile Thr Pro Gln
225 230 235 240
Arg Ser Pro Thr Gly Ala Val Glu Val Gln Val Asp Ile Glu Pro Lys
                                245 250 255
Ser Cys Asp Lys Thr His Thr Cys Pro Pro Cys Pro Ala Pro Glu Leu
                        260 265 270
Leu Gly Gly Pro Ser Val Phe Leu Phe Pro Pro Lys Pro Lys Asp Thr
                275 280 285
Leu Met Ile Ser Arg Thr Pro Glu Val Thr Cys Val Val Val Asp Val
        290 295 300
Ser His Glu Asp Pro Glu Val Lys Phe Asn Trp Tyr Val Asp Gly Val
305 310 315 320
Glu Val His Asn Ala Lys Thr Lys Pro Arg Glu Glu Gln Tyr Asn Ser
                                325 330 335
Thr Tyr Arg Val Val Ser Val Leu Thr Val Leu His Gln Asp Trp Leu
                        340 345 350
Asn Gly Lys Glu Tyr Lys Cys Lys Val Ser Asn Lys Ala Leu Pro Ala
                355 360 365
Pro Ile Glu Lys Thr Ile Ser Lys Ala Lys Gly Gln Pro Arg Glu Pro
        370 375 380
Gln Glu Tyr Thr Leu Pro Pro Ser Arg Glu Glu Met Thr Lys Asn Gln
385 390 395 400
Val Ser Leu Thr Cys Leu Val Lys Gly Phe Tyr Pro Ser Asp Ile Ala
                                405 410 415
Val Glu Trp Glu Ser Asn Gly Gln Pro Glu Asn Asn Tyr Lys Thr Thr
                        420 425 430
Pro Pro Val Leu Asp Ser Asp Gly Ser Phe Phe Leu Tyr Ser Lys Leu
                435 440 445
Thr Val Asp Lys Ser Arg Trp Gln Gln Gly Asn Val Phe Ser Cys Ser
        450 455 460
Val Met His Glu Ala Leu His Asn His Tyr Thr Gln Lys Ser Leu Ser
465 470 475 480
Leu Ser Pro Gly Lys Arg Ile Asp Tyr Lys Asp Asp Asp Asp Lys His
                                485 490 495
Val His His His His His His
                        500 503
```

LP276S and LP276ATFV2, its Fc fusion, share sequence similarity to LP276 polypeptide and other members of the human B7 family of proteins. LP276S is an analog of LP276 consisting of only two Ig-like domains. As such, LP276S and LP276ATFV2 modulate cytokines involved in inflammation and autoimmune related disorders. Accordingly, compositions comprising LP276S or LP276ATFV2 polypeptides or polynucleotides are useful for the diagnosis, treatment, and intervention of sepsis, gram negative bacteremia, inflammation, allergic autoimmune diseases, allergic responses, infectious diseases, immunodeficiencies, type 1 diabetes, Th1-dependent insulitis, pancreatitis, aberrant apoptosis, cancers, rheumatoid arthritis, eczema, psoriasis, atopy, asthma, fibrosing lung disease, acute respiratory distress syndrome (ARDS), inflammatory bowel disease, multiple sclerosis, Hashimoto's thyroiditis, Graves' disease, systemic lupus erythematosis, vasculitis, autoimmune gastritis, HIV, HIV-induced lymphoma, fulminant viral hepatitis B, fulminant viral hepatitis C, chronic hepatitis, chronic cirrhosis, liver failure, chronic glomerulonephritis, thrombotic thrombocytopenic purpura (TTP), hemolytic uremic syndrome (HUS), aplastic anemia, myelodysplasia, transplant rejection, H. pylori associated ulceration, cytoprotection during cancer treatment, recuperation during chemotherapy, recuperation from irradiation therapy, and multiple organ dysfunction syndrome (MODS).

In a most preferred embodiment, the invention provides a polypeptide comprising amino acids 29 through 701 of SEQ ID NO:4 or amino acids 29 through 487 of SEQ ID NO:8.

Definitions

The following definitions of terms are intended to correspond to those well known in the art. They are therefore not limited to the definitions given but are used according to the state of the art, as demonstrated by cited and/or contemporary publications or patents.

“Active” or “activity” for the purposes herein refers to forms of LP276 which retain at least one of the biologic and/or immunologic activities of LP276 polypeptide. Elaborating further, “biological” activity refers to a biological function (either inhibitory or stimulatory) caused by a native or naturally occurring LP276 polypeptide other than the ability to induce the production of an antibody against an antigenic epitope possessed by a native or naturally occurring LP276 polypeptide. An “immunological” activity refers only to the ability to induce the production of an antibody against an antigenic epitope possessed by a native or naturally occurring LP276 polypeptide. A preferred biological activity includes, for example, the ability to treat sepsis, gram negative bacteremia, allergic responses, allergic autoimmune diseases, type 1 diabetes, Th1-dependent insulitis, inflammation, multiple sclerosis, rheumatoid arthritis, inflammatory bowel disease, liver failure, ARDS, immunodeficiencies, cancers, or infectious diseases.

The term “amino acid” is used herein in its broadest sense and includes naturally occurring amino acids as well as non-naturally occurring amino acids, including amino acid analogs and derivatives. The latter includes molecules containing an amino acid moiety. One skilled in the art will recognize, in view of this broad definition, that reference herein to an amino acid includes, for example, naturally occurring proteogenic L-amino acids; D-amino acids; chemically modified amino acids, such as amino acid analogs and derivatives; naturally occurring non-proteogenic amino acids such as norleucine, beta-alanine, ornithine, etc.; and chemically synthesized compounds having properties known in the art to be characteristic of amino acids. As used herein, the term “proteogenic” indicates that the amino acid can be incorporated into a peptide, polypeptide, or protein in a cell through a metabolic pathway.

The term “antagonist” is used in the broadest sense and includes any molecule that partially or fully blocks, inhibits, or neutralizes biological activity of a native LP276 polypeptide disclosed herein. In a similar manner, the term “agonist” is used in the broadest sense and includes any molecule that mimics a biological activity of a native LP276 polypeptide disclosed herein. Suitable agonist or antagonist molecules specifically include agonist or antagonist antibodies or antibody fragments, fragments or amino acid sequence variants of native polypeptides, peptides, ribozymes, antisense nucleic acids, small organic molecules, etc. Methods for identifying agonists or antagonists of an LP276 polypeptide may comprise contacting an LP276 polypeptide with a candidate agonist or antagonist molecule and measuring a detectable change in one or more biological activities normally associated with the LP276 polypeptide.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules that lack antigen specificity. Polypeptides of the latter kind are, for example, produced at low levels by the lymph system and at increased levels by myelomas. The term “antibody” is used in the broadest sense and specifically covers, without limitation, intact monoclonal antibodies (MAbs), polyclonal antibodies, modified antibodies as known in the art (e.g., chimeric, humanized, recombinant, veneered, resurfaced, or CDR-grafted), anti-idiotypic (anti-id) antibodies, and antibody fragments, so long as they exhibit the desired biological activity.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), such as IgG1, IgG2, IgG3, IgG4, IgA and IgA2.

“Antibody fragments” comprise a portion of an intact antibody, preferably the antigen binding or variable region of the intact antibody. Examples of antibody fragments include Fab, Fab′, F(ab′)₂ and Fv fragments; diabodies; linear antibodies [Zapata, et al., Protein Engin. 8 (10):1057-62 (1995)]; single-chain antibody molecules; and multispecific antibodies (e.g., bispecific antibodies) formed from antibody fragments.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight polypeptides (less than about 10 residues); proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONIC®.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption but, rather, is cyclic in nature.

“Conservative substitution” or “conservative amino acid substitution” refers to a replacement of one or more amino acid residue(s) in a protein or peptide. Conservative substitutions of interest are shown in Table 10 along with preferred substitutions. If such substitutions maintain or improve the desired function, then more substantial changes, listed as exemplary substitutions in Table 10, or as further described below in reference to amino acid classes, are introduced and the products screened.

TABLE 10
Conservative Substitutions

| Original | Example | Preferred |
|---|---|---|
| Residue | Substitutions | Substitutions |
| Ala (A) | val, leu, ile | val |
| Arg (R) | lys, gln, asn | lys |
| Asn (N) | gln | gln |
| Asp (D) | glu | glu |
| Cys (C) | ser | ser |
| Gln (Q) | asn | asn |
| Glu (E) | asp | asp |
| Gly (G) | pro, ala | ala |
| His (H) | asn, gln, lys, arg | arg |
| Ile (I) | leu, val, met, ala, phe, norleucine | leu |
| Leu (L) | norleucine, ile, val, met, ala, phe | ile |
| Lys (K) | arg, gln, asn | arg |
| Met (M) | leu, phe, ile | leu |
| Phe (F) | leu, val, ile, ala, tyr | leu |
| Pro (P) | ala | ala |
| Ser (S) | thr | thr |
| Thr (T) | ser | ser |
| Trp (W) | tyr, phe | tyr |
| Tyr (Y) | trp, phe, thr, ser | phe |
| Val (V) | ile, leu, met, phe, ala, norleucine | leu |

Naturally occurring residues are divided into groups based on common side-chain properties:

(1) hydrophobic: cys, ser, thr;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404 097, WO 93/11161, and Holliger, et al., Proc. Natl. Acad. Sci. USA 90(14):6444-8 (1993).

The term “epitope tagged,” where used herein, refers to a chimeric polypeptide comprising an LP polypeptide or domain sequence thereof, fused to a “tag polypeptide.” The tag polypeptide has enough residues to provide an epitope against which an antibody may be made, or which can be identified by some other agent, yet is short enough such that it does not interfere with the activity of the LP polypeptide. The tag polypeptide preferably is also fairly unique so that the antibody does not substantially cross-react with other epitopes. Suitable tag polypeptides generally have at least six amino acid residues and usually between about eight to about fifty amino acid residues, preferably, between about ten to about twenty residues.

Papain digestion of antibodies produces two identical antigen-binding fragments, called “Fab” fragments, each with a single antigen-binding site, and a residual “Fc” fragment, a designation reflecting the ability to crystallize readily. Papain digestion provides one means of obtaining an immunoglobulin constant domain.

The Fab fragment also contains the constant domain of the light chain and the “first constant domain” (CH1) of the heavy chain. Fab fragments differ from Fv fragments by the addition of a few residues at the carboxy terminus of the heavy chain CH1 domain including one or more cystines from the antibody hinge region. Fab′-SH is the designation herein for Fab′ in which the cystine residue(s) of the constant domains bear a free thiol group. F(ab′)₂ antibody fragments were originally produced as pairs of Fab′ fragments which have hinge cystines between them. Other chemical couplings of antibody fragments are also known.

The term “fragment thereof” as used herein refers to a fragment, piece, or sub-region of a nucleic acid or protein molecule whose sequence is disclosed herein, such that the fragment comprises 5, 10, 15, 20 or more amino acids, or 15, 30, 45, 60 or more nucleotides that are contiguous in the parent protein or nucleic acid compound. When referring to a nucleic acid compound, “fragment thereof” refers to 15, 30, 45, 60 or more contiguous nucleotides, derived from the parent nucleic acid, and also, owing to the genetic code, to the complementary sequence. For example, if the fragment entails the sequence 5′-AGCTAG-3′, then “fragment thereof” would also include the complementary sequence, 3′-TCGATC-5′.

“Functional fragment” or “functionally equivalent fragment,” as used herein, refers to a region or fragment of a full-length protein or sequence of amino acids that are capable of competing with the endogenous or native LP276 polypeptide for binding to a natural or recombinantly expressed LP276 polypeptide receptor. The present invention also provides for the use of fragments of the LP276 polypeptides disclosed herein wherein said fragments retain ability to bind a natural ligand. As used herein, “functional fragments” includes fragments, whether or not fused to additional sequences, that retain and exhibit, under appropriate conditions, measurable bioactivity, for example, protection against LPS challenge in vivo. Functional fragments of the proteins disclosed herein may be produced as described herein, preferably using cloning techniques to engineer smaller versions of the functioning LP276 polypeptide, lacking sequence from the 5′ end, the 3′ end, from both ends, or from an internal site.

Functional analogs of the LP276 protein may be generated by deletion, insertion, or substitution of one or more amino acid residues. The present invention includes methods of using LP276 proteins as well as any related functional analogs that retain the ability to be employed therapeutically according to the present invention. Modifications of the amino acid sequence can generally be made in accordance with the substitutions provided in Table 10.

The term “fusion protein” denotes a hybrid protein molecule not found in nature comprising a translational fusion or enzymatic fusion in which two or more different protein segments not naturally found in a contiguous sequence are covalently linked together, generally on a single peptide chain.

“Fv” is the minimum antibody fragment that contains a complete antigen-recognition and binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDR specific for an antigen) has the ability to recognize and bind an antigen, although at a lower affinity than the entire binding site.

“HA tag,” as used herein, corresponds to an epitope derived from the influenza hemagglutinin polypeptide [Wilson, et al., Cell 37(3):767-78 (1984)]. The fusion of the HA tag to the target LP polypeptide allows easy detection and recovery of the recombinant polypeptide with an antibody that recognizes the HA epitope.

The term “HIS tag,” where used herein, refers to the LP polypeptide sequence fused to a highly-rich histidine polypeptide sequence. The HIS tag has enough histidine residues to provide a unique purification means to select for the properties of the repeated histidine residues, yet is short enough such that it does not interfere with the activity of the extracellular domain sequence of the LP polypeptide. Suitable tag polypeptides generally have at least six amino acid residues and usually between about four to about twenty amino acid residues (preferably, between about four to about ten residues, and most preferably six, such as HHHHHH). These tags consist of several codons encoding the HA or HIS tag [see, e.g., Ausubel, et al., ed., Current Protocols in Molecular Biology, John Wiley and Sons, NY (1987-1999)], followed by a termination codon and polyadenylation.

The term “homolog” or “homologous” describes the relationship between different nucleic acid compounds or amino acid sequences in which said sequences or molecules are related by partial identity or similarity at one or more blocks or regions within said molecules or sequences.

The term “host cell” as used herein refers to any eukaryotic or prokaryotic cell that is suitable for propagating and/or expressing a cloned gene contained on a vector that is introduced into said host cell by, for example, transformation or transfection, or the like.

The term “hybridization” refers to a process in which a single-stranded nucleic acid compound joins with a complementary strand through nucleotide base pairing. The degree of hybridization depends upon, for example, the degree of sequence similarity, the stringency of hybridization, and the length of hybridizing strands. “Selective hybridization” refers to hybridization under conditions of high stringency.

The term “immunoadhesin,” also referred to as an Fc fusion, designates antibody-like molecules that combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand.

Administration “in combination with” one or more additional therapeutic agents includes simultaneous (concurrent) and consecutive administration in any order.

“Isolated,” when used to describe the various polypeptides or polynucleotides disclosed herein, means a polypeptide or polynucleotide that has been identified and separated and/or recovered from a component of its natural environment. Preferably, the isolated polypeptide or polynucleotide is free of association with all components with which it is naturally associated. Contaminant components of its natural environment are materials that would typically interfere with diagnostic, prophylactic, or therapeutic uses for the polypeptide or polynucleotide and may include enzymes, hormones, and other proteinaceous or non-proteinaceous solutes. In preferred embodiments, the polypeptide or polynucleotide will be purified (1) to greater than 95% purity by weight of polypeptide or polynucleotide as determined by the Lowry method, and most preferably more than 99% by weight, (2) to a degree sufficient to obtain at least fifteen residues of N-terminal or internal amino acid sequence by use of a spinning cup sequenator, or (3) to homogeneity by SDS-PAGE under reducing or nonreducing conditions using Coomassie blue, or preferably, silver stain. Isolated polypeptide or polynucleotide includes polypeptide or polynucleotide in situ within recombinant cells, since at least one component of the LP polypeptide or polynucleotide's natural environment will not be present. Ordinarily, however, isolated polypeptide or polynucleotide will be prepared by at least one purification step.

A “liposome” is a small vesicle, composed of various types of lipids, phospholipids and/or surfactants, which is useful for delivery of a drug (such as an LP276 polypeptide) to a mammal. The components of the liposome are commonly arranged in a bilayer formation, similar to the lipid arrangement of biological membranes.

The polynucleotides of the present utility invention are designated herein as “LP polynucleotide(s)” or “LP polypeptide-encoding polynucleotide(s).” The polypeptides of the present invention are designated herein as “LP polypeptide(s)” or “LP protein(s).” When immediately followed by a numerical designation (e.g., LP276), the term “LP” refers to a specific group of molecules as defined herein. A complete designation, wherein the term “LP” is immediately followed by a numerical designation plus a molecule type (e.g., LP276 polypeptide or LP276L polynucleotide), refers to a specific type of molecule within the designated group of molecules as defined herein.

The LP molecules described herein may be isolated from a variety of sources including, but not limited to, human tissue types, or prepared by recombinant or synthetic methods.

The LP polynucleotide can be composed of any polyribonucleotide or polydeoxyribonucleotide, which may be unmodified RNA or DNA or modified RNA or DNA. For example, the LP polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the LP polynucleotides can be composed of triple-stranded regions comprising RNA or DNA or both RNA and DNA. LP polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The term “LP276 polypeptide” specifically encompasses truncated or secreted forms of an LP276 polypeptide (e.g., soluble forms containing, for instance, an extracellular domain sequence), variant forms (e.g., alternatively spliced forms), and allelic variants of an LP276 polypeptide.

In one embodiment, the native sequence LP276 polypeptide is a full-length or mature LP276 polypeptide comprising amino acids 1 or about 29 through 534 of SEQ ID NO:2. Also, while the LP276 polypeptides disclosed herein are shown to begin with a methionine residue designated as amino acid position 1, it is conceivable and possible that another methionine residue located either upstream or downstream from amino acid position 1 may be employed as the starting amino acid residue.

In a preferred embodiment, “LP276ATFV polypeptide” or “LP276ATFV protein” is an Fc fusion of the LP276L polypeptide with IgG1, a Flag epitope tag, and a HIS tag, comprising amino acids 1 or about 29 through about 717, inclusive, of SEQ ID NO:4.

In another preferred embodiment, “LP276ATFV2 polypeptide” or “LP276ATFV2 protein” is an Fc fusion of the LP276S polypeptide with IgG1, a Flag epitope tag, and a HIS tag, comprising amino acids 1 or about 29 through about 503, inclusive, of SEQ ID NO:8.

In a most preferred embodiment, “LP276ATFV” or “LP276ATFV2” is an Fc fusion without the Flag epitope tag or HIS tag, comprising amino acids 1 or about 29 through about 701 of SEQ ID NO:4, or amino acids 1 or about 29 through about 487 of SEQ ID NO:8, respectively.

In another preferred embodiment, “LP276L polypeptide” is a fragment of the native LP276 polypeptide, comprising amino acids 1 or about 29 through about 465, inclusive, of SEQ ID NO:2.

In another embodiment, “LP276S polypeptide” is an analog of the native LP276 polypeptide, comprising amino acids 1 or about 29 through about 315, inclusive, of SEQ ID NO:4.

LP sequences can be isolated from nature or can be produced by recombinant or synthetic means. LP polypeptides include, but are not limited to, deglycosylated, unglycosylated, and modified glycosylated forms of LP276 polypeptides, as well as sufficiently homologous forms having conservative substitutions, additions, or deletions of the amino acid sequence, as well as portions thereof such that the molecule retains LP276-like functionality and bioactivity.

The terms “LP276 polypeptide(s),” “LP276L polypeptide(s),” “LP276S polypeptide(s),” “LP276ATFV polypeptide(s),” or “LP276ATFV2 polypeptide(s)” are also meant to encompass polypeptides containing pro-, or prepro-sequences, that when processed result in the production of the respective LP polypeptide.

An “LP variant polynucleotide” or “LP variant nucleic acid sequence” means an active LP polypeptide-encoding nucleic acid molecule as defined below, having at least about 75% nucleic acid sequence identity with SEQ ID NO:1, 3, 5, or 7. Ordinarily, an LP variant polynucleotide will have at least about 75% nucleic acid sequence identity, more preferably at least about 80% nucleic acid sequence identity, yet more preferably at least about 81% nucleic acid sequence identity, yet more preferably at least about 82% nucleic acid sequence identity, yet more preferably at least about 83% nucleic acid sequence identity, yet more preferably at least about 84% nucleic acid sequence identity, yet more preferably at least about 85% nucleic acid sequence identity, yet more preferably at least about 86% nucleic acid sequence identity, yet more preferably at least about 87% nucleic acid sequence identity, yet more preferably at least about 88% nucleic acid sequence identity, yet more preferably at least about 89% nucleic acid sequence identity, yet more preferably at least about 90% nucleic acid sequence identity, yet more preferably at least about 91% nucleic acid sequence identity, yet more preferably at least about 92% nucleic acid sequence identity, yet more preferably at least about 93% nucleic acid sequence identity, yet more preferably at least about 94% nucleic acid sequence identity, yet more preferably at least about 95% nucleic acid sequence identity, yet more preferably at least about 96% nucleic acid sequence identity, yet more preferably at least about 97% nucleic acid sequence identity, yet more preferably at least about 98% nucleic acid sequence identity, yet more preferably at least about 99% nucleic acid sequence identity with the nucleic acid sequences shown in SEQ ID NO:1, 3, 5, or 7. Variants specifically exclude or do not encompass the native nucleotide sequence, as well as those prior art sequences that share 100% identity with the nucleotide sequences of the invention.

“LP variant polypeptide” or “LP variant” means an “active” LP polypeptide or fragment thereof as defined herein, having at least about 90% amino acid sequence identity with the LP polypeptides having the deduced amino acid sequences as shown in SEQ ID NO:2, 4, 6, or 8. Such LP polypeptide variants include, for instance, LP276 polypeptides wherein one or more amino acid residues are added, substituted or deleted, at the N- or C-terminus or within the sequence of SEQ ID NO:2. Ordinarily, an LP polypeptide variant will have at least about 90% amino acid sequence identity, preferably at least about 91% sequence identity, yet more preferably at least about 92% sequence identity, yet more preferably at least about 93% sequence identity, yet more preferably at least about 94% sequence identity, yet more preferably at least about 95% sequence identity, yet more preferably at least about 96% sequence identity, yet more preferably at least about 97% sequence identity, yet more preferably at least about 98% sequence identity, yet more preferably at least about 99% amino acid sequence identity with the amino acid sequence described, with or without the signal peptide.

Similarly, LP polynucleotides or polypeptides useful to practice the present invention may additionally contain other non-LP polynucleotide or polypeptide sequences, respectively, provided that the polypeptide encoded thereby still retains a functional activity. More specifically, LP polypeptides useful in practicing the present invention also include chimeric protein molecules not found in nature comprising a translational fusion, or in some cases an enzymatic fusion, in which two or more different proteins or fragments thereof are covalently linked on a single polypeptide chain. A preferred LP polypeptide for practicing the present invention comprises at least one functional fragment of the full-length LP276 polypeptide as shown in SEQ ID NO:2 and at least one effector function of an immunoglobulin constant domain. The fusion molecules are a subclass of chimeric polypeptide fusions of LP276 polypeptides that additionally contain a portion of an immunoglobulin sequence (herein referred to as “LP-Ig”). The chimeric LP-Ig fusions may also comprise forms in monomeric, homo- or heteromultimeric, and particularly homo- or heterodimeric, or homo- or heterotetrameric forms. Optionally, the chimeras may be in dimeric forms or homodimeric heavy chain forms. Tetrameric forms containing a four chain structural unit are the natural forms in which IgG, IgD, and IgE occur. A four-chain structure may also be repeated. Different chimeric forms containing a native immunoglobulin are known in the art (WO 98/25967). The mature human protein of Example 8 is exemplary of an “LP276-Ig.” As used herein, the term “LP276-Ig” designates antibody-like molecules that combine at least one LP276 domain with the effector functions of immunoglobulin constant domain. The immunoglobulin constant domain sequence may be obtained from any immunoglobulin, such as IgG1, IgG2, IgG3 or IgG4 subtypes, IgA (including IgA1 and IgA2), IgE, IgD or IgM. Preferred fusions contain the LP276 fragment fused to the carboxyl terminus of the Ig region. However, fusions of an LP276 polypeptide or fragment thereof to the N-terminus of the Ig region are also contemplated. LP276 fusion polypeptides can also comprise additional amino acid residues, such as affinity tags that aid in the purification or identification of the molecule or provide sites of attachment to a natural ligand.

The term “mature protein” or “mature polypeptide” as used herein refers to the form(s) of the protein produced by expression in a mammalian cell. It is generally hypothesized that once export of a growing protein chain across the rough endoplasmic reticulum has been initiated, proteins secreted by mammalian cells have a signal sequence which is cleaved from the complete polypeptide to produce a “mature” form of the protein. Oftentimes, cleavage of a secreted protein is not uniform and may result in more than one species of mature protein. The cleavage site of a secreted protein is determined by the primary amino acid sequence of the complete protein and generally cannot be predicted with complete accuracy. Methods for predicting whether a protein has a signal peptide sequence, as well as the cleavage point for that sequence, are available. A cleavage point may exist within the N-terminal domain between amino acid 10 and amino acid 35. More specifically the cleavage point is likely to exist after amino acid 15 but before amino acid 30, more likely after amino acid 27. As one of ordinary skill would appreciate, however, cleavage sites sometimes vary from organism to organism and cannot be predicted with absolute certainty. Optimally, cleavage sites for a secreted protein are determined experimentally by N-terminal sequencing of the one or more species of mature proteins found within a purified preparation of the protein.

The term “modulate” means to affect (e.g., either upregulate, downregulate, or otherwise control) the level of a signaling pathway. Cellular processes under the control of signal transduction include, but are not limited to, transcription of specific genes, normal cellular functions, such as metabolism, proliferation, differentiation, adhesion, apoptosis and survival, as well as abnormal processes, such as transformation, blocking of differentiation and metastasis.

A “nucleic acid probe” or “probe” as used herein is a labeled nucleic acid compound that hybridizes with another nucleic acid compound. “Nucleic acid probe” means a single stranded nucleic acid sequence that will combine with a complementary or partially complementary single stranded target nucleic acid sequence to form a double-stranded molecule. A nucleic acid probe may be an oligonucleotide or a nucleotide polymer. A probe will usually contain a detectable moiety that may be attached to the end(s) of the probe or be internal to the sequence of the probe,

Nucleic acid is “operably linked” when it is placed into a functional relationship with another nucleic acid sequence. For example, DNA for a presequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “patient” as used herein refers to any mammal, including humans, domestic and farm animals, zoo, sports or pet animals, such as cattle (e.g., cows), horses, dogs, sheep, pigs, rabbits, goats, cats, and non-domesticated animals like mice and rats. In a preferred embodiment of the invention, the mammal is a human or mouse.

“Percent (%) amino acid sequence identity” with respect to the LP amino acid sequences identified herein is defined as the percentage of amino acid residues in a candidate sequence that are identical with the amino acid residues in an LP polypeptide sequence, after aligning the sequences and introducing gaps, if necessary, to achieve the maximum percent sequence identity, and not considering any conservative amino acid substitutions as part of the sequence identity. Alignment for purposes of determining percent amino acid sequence identity can be achieved in various ways that are within the skill in the art, for instance, using publicly available computer software such as ALIGN, ALIGN-2, Megalign (DNASTAR) or BLAST (e.g., Blast, Blast-2, WU-Blast-2) software. Those skilled in the art can determine appropriate parameters for measuring alignment, including any algorithms needed to achieve maximal alignment over the full length of the sequences being compared. The percent identity values used herein are generated using WU-BLAST-2 [Altschul and Gish, Meth. Enzymol. 266: 460-80 (1996)]. Most of the WU-BLAST-2 search parameters are set to the default values. Those not set to default values, i.e., the adjustable parameters, are set with the following values: overlap span=1; overlap fraction=0.125; word threshold (T)=11; and scoring matrix=BLOSUM 62. For purposes herein, a percent amino acid sequence identity value is determined by dividing (a) the number of matching identical amino acid residues between the amino acid sequence of the LP polypeptide of interest and the comparison amino acid sequence of interest (i.e., the sequence against which the LP polypeptide of interest is being compared) as determined by WU-BLAST-2, by (b) the total number of amino acid residues of the LP polypeptide of interest, respectively.

“Percent (%) nucleic acid sequence identity” with respect to the LP polynucleotide sequences identified herein is defined as the percentage of nucleotides in a candidate sequence that are identical with the nucleotides in the LP polynucleotide sequence after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as ALIGN, Align-2, Megalign (DNASTAR), or BLAST (e.g., Blast, Blast-2) software. Those skilled in the art can determine appropriate parameters for measuring alignment, including any algorithms needed to achieve maximal alignment over the full length of the sequences being compared. For purposes herein, however, percent nucleic acid identity values are generated using the WU-BLAST-2 (BlastN module) program [Altschul and Gish, Meth. Enzymol. 266: 460-80 (1996)]. Most of the WU-BLAST-2 search parameters are set to the default values. Those not set default values, i.e., the adjustable parameters, are set with the following values: overlap span=1; overlap fraction=0.125; word threshold (T)=11; and scoring matrix=BLOSUM62. For purposes herein, a percent nucleic acid sequence identity value is determined by dividing (a) the number of matching identical nucleotides between the nucleic acid sequence of the LP polypeptide-encoding nucleic acid molecule of interest and the comparison nucleic acid molecule of interest (i.e., the sequence against which the LP polypeptide-encoding nucleic acid molecule of interest is being compared) as determined by WU-BLAST-2, by (b) the total number of nucleotides of the LP polypeptide-encoding nucleic acid molecule of interest.

“Pharmaceutically acceptable salt” includes, but is not limited to, salts prepared with inorganic acids, such as chloride, sulfate, phosphate, diphosphate, hydrobromide, and nitrate salts, or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

The term “plasmid” refers to an extrachromosomal genetic element. The plasmids disclosed herein are commercially available, publicly available on an unrestricted basis, or can be constructed from readily available plasmids in accordance with published procedures.

The term “positives,” in the context of sequence comparison performed as described above, includes residues in the sequences compared that are not identical but have similar properties (e.g., as a result of conservative substitutions). The percent identity value of positives is determined by the fraction of residues scoring a positive value in the BLOSUM 62 matrix. This value is determined by dividing (a) the number of amino acid residues scoring a positive value in the BLOSUM62 matrix of WU-BLAST-2 between the LP polypeptide amino acid sequence of interest and the comparison amino acid sequence (i.e., the amino acid sequence against which the LP polypeptide sequence is being compared) as determined by WU-BLAST-2, by (b) the total number of amino acid residues of the LP polypeptide of interest.

A “portion” of an LP polypeptide sequence is at least about 20, 25, 30, 35, 40, 45, 50, 55, 60, 65, 70, 75, 80, 85, 90, 95, or 100 contiguous amino acid residues in length.

A “primer” is a nucleic acid fragment which functions as an initiating substrate for enzymatic or synthetic elongation of, for example, a nucleic acid compound.

The term “promoter” refers to a nucleic acid sequence that directs transcription, for example, of DNA to RNA. An inducible promoter is one that is regulatable by environmental signals, such as carbon source, heat, or metal ions, for example. A constitutive promoter generally operates at a constant level and is not regulatable.

The term “recombinant DNA expression vector” or “expression vector” as used herein refers to any recombinant DNA cloning vector (such as a plasmid or phage), in which a promoter and other regulatory elements are present, thereby enabling transcription of an inserted DNA, which may encode a polypeptide.

“Single-chain Fv” or “sFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domain, which enables the sFv to form the desired structure for antigen binding. For a review of sFv, see Pluckthun, The Pharmacology of Monoclonal Antibodies, vol. 113, Rosenburg and Moore, eds., Springer-Verlag, New York, pp. 269-315 (1994).

A “small molecule” is defined herein to have a molecular weight below about 500 daltons.

“Stringency” of hybridization reactions is readily determinable by one of ordinary skill in the art, and generally is an empirical calculation dependent upon probe length, washing temperature, and salt concentration. In general, longer nucleic acid probes required higher temperatures for proper annealing, while shorter nucleic acid probes need lower temperatures. Hybridization generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reactions more stringent, while lower temperatures less so. For additional details and explanation of stringency of hybridization reactions, see Ausubel, et al., Current Protocols in Molecular Biology, Wiley Interscience Publishers, 1995.

“Stringent conditions” or “high stringency conditions”, as defined herein, may be identified by those that (1) employ low ionic strength and high temperature for washing, for example, 15 mM sodium chloride/1.5 mM sodium citrate/0.1% sodium dodecyl sulfate at 50 degrees C.; (2) employ during hybridization a denaturing agent, such as formamide, for example, 50% (v/v) formamide with 0.1% bovine serum albumin/0.1% ficoll/0.1% polyvinylpyrrolidone/50 mM sodium phosphate buffer at pH 6.5 with 750 mM sodium chloride/75 mM sodium citrate at 42 degrees C.; or (3) employ 50% formamide, 5× SSC (750 mM sodium chloride, 75 mM sodium citrate), 50 mM sodium phosphate (pH 6.8), 0.1% sodium pyrophosphate, 5× Denhardt's solution, sonicated salmon sperm DNA (50 μg/mL), 0.1% SDS, and 10% dextran sulfate at 42 degrees C. with washes at 42 degrees C. in 0.2× SSC (30 mM sodium chloride/3 mM sodium citrate) and 50% formamide at 55 degrees C., followed by a high-stringency wash consisting of 0.1× SSC containing EDTA at 55 degrees C. “Moderately stringent conditions” may be identified as described by Sambrook, et al. [Molecular Cloning: A Laboratory Manual, New York: Cold Spring Harbor Press, (1989)], and include the use of washing solution and hybridization conditions (e.g., temperature, ionic strength and % SDS) less stringent than those described above. An example of moderately stringent conditions is overnight incubation at 37 degrees C. in a solution comprising: 20% formamide, 5× SSC (750 mM sodium chloride, 75 mM sodium citrate), 50 mM sodium phosphate at pH 7.6, 5× Denhardt's solution, 10% dextran sulfate, and 20 mg/mL denatured sheared salmon sperm DNA, followed by washing the filters in 1× SSC at about 37 to 50 degrees C. The skilled artisan will recognize how to adjust the temperature, ionic strength, etc., as necessary to accommodate factors such as probe length and the like.

“Substantially pure,” when used in reference to an LP polynucleotide or polypeptide means that said “LP276,” “LP276L,” “LP276S,” “LP276ATFV,” or “LP276ATFV2” is separated from other cellular and noncellular molecules, including other proteins, lipids, carbohydrates or other materials with which it is naturally associated when produced recombinantly or synthesized without any general purifying steps. A “substantially pure” LP polypeptide described herein could be prepared by a variety of techniques well known to the skilled artisan, including, for example, the described methods of LP polypeptide purification referred to or described herein. In preferred embodiments, the LP polypeptide will be purified (1) to greater than 95% purity by weight of the LP polypeptide to the weight of total protein as determined by the Lowry method, and most preferably more than 99% by weight to the weight of total protein, (2) to a degree sufficient to obtain at least fifteen residues of N-terminal or internal amino acid sequence by use of a spinning cup sequenator, or (3) to apparent homogeneity by SDS-PAGE under reducing or nonreducing conditions using Coomassie Blue, or preferably, silver stain, such that the major band constitutes at least 95%, and more preferably 99%, of the stained protein observed on the gel.

The term “symptom” in reference to sepsis, gram negative bacteremia, allergic responses, allergic autoimmune diseases, type 1 diabetes, Th1-dependent insulitis, immunodeficiencies, cancers, inflammation, or infectious diseases. Infectious disease is meant to include, but not limited to, one or more of the following: chills, profuse sweating, fever, weakness, hypotension, leukopenia, intravascular coagulation, shock, respiratory distress, organ failure, prostration, ruffled fur, diarrhea, eye exudate, and death, alone or in combination. This list is not meant to be exclusive, but may be supplemented with symptoms or combinations of symptoms that a person of ordinary skill would recognize as associated with sepsis, gram negative bacteremia, allergic responses, allergic autoimmune diseases, type 1 diabetes, Th1-dependent insulitis, immunodeficiencies, cancers, inflammation, or infectious diseases. Symptoms associated with sepsis, gram negative bacteremia, allergic responses, allergic autoimmune diseases, type 1 diabetes, Th1-dependent insulitis, immunodeficiencies, cancers, inflammation, or infectious diseases that are treatable with LP polypeptides are within the scope of this invention. A symptom associated with sepsis, gram negative bacteremia, allergic responses, allergic autoimmune diseases, type 1 diabetes, Th1-dependent insulitis, immunodeficiencies, cancers, inflammation, or infectious diseases may also be associated with another condition.

A “therapeutically-effective amount” is the minimal amount of active agent (e.g., an LP276 polypeptide, antagonist or agonist thereof) which is necessary to impart therapeutic benefit or desired biological effect to a patient. For example, a “therapeutically-effective amount” to a mammal suffering from sepsis is such an amount which induces, ameliorates or otherwise causes an improvement in the pathological symptoms, disease progression, physiological conditions associated with, or resistance to succumbing to a disorder principally characterized by immunodeficiency, cancer, inflammation, and/or infectious disease when the LP polypeptide is administered. The precise amount of LP polypeptide administered to a particular patient will depend upon numerous factors, e.g., such as the specific binding activity of the molecule, the delivery device employed, physical characteristics, its intended use, and patient considerations, and can readily be determined by one skilled in the art, based upon the information provided herein and that which is known in the art.

The terms “treating,” “treatment,” and “therapy” as used herein refer to curative therapy, prophylactic therapy, and preventive therapy. An example of “preventive therapy” is the prevention or lessening of a targeted disease or related condition thereto. Those in need of treatment include those already with the disease or condition as well as those prone to have the disease or condition is to be prevented. The terms “treating”, “treatment”, and “therapy” as used herein also describe the management and care of a patient for the purpose of combating a disease or related condition, and includes the administration of LP276, LP276L, LP276S, LP276ATFV, or LP276ATFV2 to alleviate the symptoms or complications of said disease or condition.

The term “vector” as used herein refers to a nucleic acid compound used for introducing exogenous or endogenous DNA into host cells. A vector comprises a nucleotide sequence that may encode one or more protein molecules. Plasmids, cosmids, viruses, and bacteriophages, in the natural state or which have undergone recombinant engineering, are examples of commonly used vectors.

The various restriction enzymes disclosed and described herein are commercially available and the manner of use of said enzymes including reaction conditions, cofactors, and other requirements for activity are well known to one of ordinary skill in the art. Reaction conditions for particular enzymes are carried out according to the manufacturer's recommendation.

Protein Synthesis

Skilled artisans will recognize that the LP polypeptides utilized in the embodiments of the present invention can be synthesized by a number of different methods, such as chemical methods well known in the art, including solid phase peptide synthesis or recombinant methods. Both methods are described in U.S. Pat. No. 4,617,149, incorporated herein by reference.

The principles of solid phase chemical synthesis of polypeptides are well known in the art and may be found in general texts in the area. See, e.g., Dugas and Penney, Bioorganic Chemistry, Springer-Verlag, NY, 54-92 (1981). For example, peptides may be synthesized by solid-phase methodology utilizing an Applied Biosystems 430A peptide synthesizer (Applied Biosystems, Foster City, Calif.) and synthesis cycles supplied by Applied Biosystems.

The proteins utilized in the present invention can also be produced by recombinant DNA methods using the LP polynucleotide sequences provided herein. Recombinant methods are preferred if a high yield is desired. Expression of the LP polypeptide can be carried out in a variety of suitable host cells, well known to those skilled in the art. For this purpose, the LP polynucleotide constructs are introduced into a host cell by any suitable means, well known to those skilled in the art. Chromosomal integration of LP276 expression vectors are within the scope of the present invention, as well as suitable extra-chromosomally maintained expression vectors so that the coding region of the LP polynucleotide is operably-linked to a constitutive or inducible promoter.

The basic steps in the recombinant production of LP proteins are:

a) constructing a recombinant, synthetic or semi-synthetic DNA encoding an LP protein;

b) integrating said DNA into an expression vector in a manner suitable for expressing the LP protein;

c) transforming or otherwise introducing said vector into an appropriate eukaryotic or prokaryotic host cell forming a recombinant host cell;

d) culturing said recombinant host cell in a manner to express the LP protein; and

e) recovering and substantially purifying the LP protein by any suitable means well known to those skilled in the art.

Production of LP proteins also include routes where direct chemical synthetic procedures are employed as well as products produced by recombinant techniques from a eukaryotic host, including, for example, yeast, higher plant, insect and mammalian cells. Depending upon the host employed in a recombinant production procedure, the polypeptides of the present invention can be glycosylated or non-glycosylated. Additionally, the amino acid sequence of an LP polypeptide may optionally include a conservative substitution. Preferred LP molecules are glycosylated as would occur in eukaryotic hosts. In addition, the LP polypeptides of the invention can also include an initial modified methionine residue, in some cases as a result of host-mediated processes. Such methods are described in many standard laboratory manuals, such as Sambrook, et al., Molecular Cloning: A Laboratory Manual, Cold Spring Laboratory Press, Cold Spring Harbor, N.Y. (1989), Chapters 17.37-17.42; Ausubel, supra, Chapters 10, 12, 13, 16, 18 and 20, entirely incorporated herein by reference.

Those skilled in the art will recognize that owing to the degeneracy of the genetic code (i.e., sixty-four codons which encode twenty amino acids), numerous “silent” substitutions of nucleotide base pairs could be introduced into an LP polynucleotide sequence without altering the identity of the encoded amino acid(s) or protein product. Use of all such substituted LP molecules is intended to be within the scope of the present invention.

Fragments of the proteins disclosed herein may be generated by any number of suitable techniques, including chemical synthesis. For instance, constant regions of immunoglobulins can be obtained by papain digestion of antibodies. Alternatively, recombinant DNA mutagenesis techniques can provide LP molecules [see, e.g., Struhl, “Reverse biochemistry: Methods and applications for synthesizing yeast proteins in vitro,” Meth. Enzymol. 194:520-35]. For example, a nested set of deletion mutations are introduced into an LP polypeptide-encoding polynucleotide such that varying amounts of the protein coding region are deleted, either from the amino terminal end, or from the carboxyl end of the protein molecule. Further, additional changes or additions to the molecule can be made. This method can also be used to create internal fragments of the intact protein in which both the carboxyl and/or amino terminal ends are removed. Several appropriate nucleases can be used to create such deletions, for example Bal31, or in the case of a single stranded nucleic acid compound, mung bean nuclease. For simplicity, it is preferred that the intact LP gene be cloned into a single-stranded cloning vector, such as bacteriophage M13 or equivalent. If desired, the resulting gene deletion fragments can be subcloned into any suitable vector for propagation and expression of said fragments in any suitable host cell.

LP polypeptide can additionally be fused to a marker protein or an epitope tag. Such fusions include, but are not limited to, fusions to an enzyme, fluorescent protein, or luminescent protein which provides a marker function; or fusions to any amino acid sequence which can be employed for purification of the polypeptide or a proprotein sequence.

Methods of constructing fusion proteins (chimeras) composed of the binding domain of one protein and the constant region of an immunoglobulin (herein designated as “LP-Ig”) are generally known in the art. For example, chimeras containing the Fc region of human IgG and the binding region of other protein receptors are known in the art for chimeric antibodies. LP-Ig structures of the present invention can be constructed using methods similar to the construction of chimeric antibodies. In chimeric antibody construction, the variable domain of one antibody of one species is substituted for the variable domain of another species [see EP 0 125 023; EP 173 494; Munro, Nature 312(5995):597 (1984); Neuberger, et al., Nature 312(5995):604-8 (1984); Sharon, et al., Nature 309(5966):364-7 (1984); Morrison and Oi, Annu. Rev. Immunol. 2:239-56 (1984); Morrison, Science 229(4719):1202-7 (1985); Boulianne, et al., Nature 312(5995):643-6 (1984); Capon, et al., Nature 337(6207):525-31(1989); Traunecker, et al., Nature 339(6219):68-70 (1989)]. Here, a functional domain of the LP276 polypeptide is substituted for the variable domain of the recipient antibody structure.

Generally, methods for constructing LP fusion proteins include use of recombinant DNA technology. The DNA encoding a functional domain can optionally be fused with additional domains or segments of the LP polypeptide or with an Ig constant region. A polynucleotide encoding any domain of an LP polypeptide can be obtained by PCR or by restriction enzyme cleavage. This DNA fragment is readily inserted proximal to DNA encoding an immunoglobulin light or heavy chain constant region and, if necessary, the resulting construct is tailored by mutagenesis, to insert, delete, or change the codon sequence. Preferably, the selected immunoglobulin region is a human immunoglobulin region when the chimeric molecule is intended for in vivo therapy for humans. Most preferably, the selected immunoglobulin region is an IgG region. DNA encoding immunoglobulin light or heavy chain constant regions are known or readily available from cDNA libraries or can be synthesized. See, for example, Adams, et al., Biochemistry 19(12):2711-9 (1980); Gough, et al., Biochemistry 19(12):2702-10 (1980); Dolby, et al., Proc. Natl. Acad. Sci. USA 77(10):6027-31 (1980); Rice and Baltimore, Proc. Natl. Acad. Sci. USA 79(24):7862-5 (1982); Falkner and Zachau, et al., Nature 298(5871):286-8 (1982); and Morrison and Oi, Annu. Res. Immunol. 2:239-56 (1984). Other teachings of preparing chimeric molecules are known from the preparation of immunoadhesin chimeras, such as CD4-Ig [Capon, et al., Nature 337(6207):525-31 (1989); Byrn, et al., Nature, 344(6267):667-70 (1990)] and TNFR chimeras, such as TNFR-IgG [Ashkenazi, et al., Proc. Natl. Acad. Sci. 88(23):10535-9 (1991); Peppel, et al., J. Cell. Biochem. Supp. 15F-P439:118 (1991)].

Protein Purification

Generally, LP polypeptides are produced recombinantly. Once expressed, they can be isolated from the cells by applying standard protein isolation techniques to the lysates or purified from the media. The monitoring of the purification process can be accomplished by using standard Western blot techniques or radioimmunoassays or other standard immunoassay techniques.

LP polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, size exclusion chromatography, and lectin chromatography. Preferably, high performance liquid chromatography (“HPLC”), cation exchange chromatography, affinity chromatography, size exclusion chromatography, or combinations thereof, are employed for purification. Particular methods of using protein A or protein G chromatography for purification are known in the art and are particularly applicable where the LP polypeptide contains an immunoglobulin Fc region. Protein A and protein G binds the Fc regions of IgG antibodies and, therefore, makes a convenient tool for the purification of LP polypeptides containing the IgG region. LP polypeptide purification is meant to include purified parts of the chimera (the extracellular region and the immunoglobulin constant region) that are purified separately and then combined by disulfide bonding, cross-linking or the like.

The purification of LP polypeptides can be accomplished by a number of special techniques known in the art that take particular advantage of structural and functional features of these molecules. See, e.g., Kwon, et al., J. Biol. Chem. 272(22):14272-6 (1997); Emery, et al., J. Biol. Chem. 273(42):14363-7 (1998); Harrop, et al., J. Biol. Chem. 273(42):27548-56 (1998); Harrop, et al., J. Immunol. 161(4): 1786-94 (1998). Further, a number of advantageous protein sequences can be incorporated into the LP polypeptide produced, such as factor Xa cleavage sites, a HIS tag sequence, or the incorporation of specific epitopes, as is known in the art.

Expressing Recombinant LP Proteins in Host Cells

Prokaryotes may be employed in the production of recombinant LP proteins. For example, the Escherichia coli K12 strain 294 (ATCC 31446) is particularly useful for the prokaryotic expression of foreign proteins. Other strains of E. coli, bacilli such as Bacillus subtilis, enterobacteriaceae such as Salmonella typhimurium or Serratia marcescens, various Pseudomonas species and other bacteria, such as Streptomyces, may also be employed as host cells in the cloning and expression of the recombinant proteins of this invention.

Promoter sequences suitable for driving the expression of genes in prokaryotes include beta-lactamase (e.g., vector pGX2907, ATCC 39344, contains a replicon and beta-lactamase gene), lactose systems [Chang, et al., Nature (London) 275:615 (1978); Goeddel, et al., Nature (London) 281:544 (1979)], alkaline phosphatase, and the tryptophan (trp) promoter system (vector pATH1, ATCC 37695), which is designed to facilitate expression of an open reading frame as a trpE fusion protein under the control of the trp promoter. Hybrid promoters such as the tac promoter (isolatable from plasmid pDR540, ATCC 37282) are also suitable. Still other bacterial promoters, whose nucleotide sequences are generally known, may be ligated to DNA encoding the protein of the instant invention, using linkers or adapters to supply any required restriction sites. Promoters for use in bacterial systems also will contain a Shine-Dalgarno sequence operably linked to the DNA encoding the desired polypeptides. These examples are illustrative rather than limiting.

The LP proteins required to practice the present invention may be synthesized either by direct expression or as a fusion protein comprising the protein of interest as a translational fusion with another protein or peptide that may be removed by enzymatic or chemical cleavage. It is often observed in the production of certain peptides in recombinant systems that expression as containing other desired sequences prolongs the lifespan, increases the yield of the desired peptide, or provides a convenient means of isolating the protein. This is particularly relevant when expressing mammalian proteins in prokaryotic hosts. A variety of peptidases (e.g., enterokinase and thrombin) which cleave a polypeptide at specific sites or digest the peptides from the amino or carboxy termini (e.g., diaminopeptidase) of the peptide chain are known. Furthermore, particular chemicals (e.g., cyanogen bromide) will cleave a polypeptide chain at specific sites. The skilled artisan will appreciate the modifications necessary to the amino acid sequence (and synthetic or semi-synthetic coding sequence if recombinant means are employed) to incorporate site-specific internal cleavage sites. See e.g., Carter, “Site Specific Proteolysis of Fusion Proteins”, Chapter 13, in Protein Purification: From Molecular Mechanisms to Large Scale Processes, American Chemical Society, Washington, DC (1990).

In addition to prokaryotes, a variety of amphibian expression systems, such as frog oocytes, and mammalian cell systems can be used. The choice of a particular host cell depends to some extent on the particular expression vector used. Exemplary mammalian host cells suitable for use in the present invention include 293 (e.g., ATCC CCL 1573), HepG-2 (ATCC HB 8065), CV-1 (ATCC CCL 70), LC-MK2 (ATCC CCL 7.1), 3T3 (ATCC CCL 92), CHO-K1 (ATCC CCL 61), HeLa (ATCC CCL 2), RPMI8226 (ATCC CCL 155), H4IIEC3 (ATCC CCL 1600), C127I (ATCC CCL 1616), HS-Sultan (ATCC CCL 1484), and BHK-21 (ATCC CCL 10), for example.

A wide variety of vectors are suitable for transforming mammalian host cells. For example, the pSV2-type vectors comprise segments of the simian virus 40 (SV40) genome required for transcription and polyadenylation. A large number of plasmid pSV2-type vectors have been constructed, such as pSV2-gpt, pSV2-neo, pSV2-dhfr, pSV2-hyg, and pSV2-beta-globin, in which the SV40 promoter drives transcription of an inserted gene. These vectors are widely available from sources such as the American Type Culture Collection (ATCC), Rockville, Md., or the National Center for Agricultural Utilization Research, Peoria, Ill.

Promoters suitable for expression in mammalian cells include the SV40 late promoter, promoters from eukaryotic genes, such as, for example, the estrogen-inducible chicken ovalbumin gene, the interferon genes, the glucocorticoid-inducible tyrosine aminotransferase gene, the thymidine kinase gene promoter, and the promoters of the major early and late adenovirus genes.

Plasmid pRSVcat (ATCC 37152) comprises portions of a long terminal repeat of the Rous Sarcoma virus, a virus known to infect chickens and other host cells. This long terminal repeat contains a promoter that is suitable for this use. [Gorman, et al., Proc. Nat. Acad. Sci. USA 79(22):6777-81 (1982)]. The plasmid pMSVi (NRRL B-15929) comprises the long terminal repeats of the Murine Sarcoma virus, a virus known to infect mouse and other host cells. The mouse metallothionein promoter has also been well characterized for use in eukaryotic host cells and is suitable for use in the present invention. This promoter is present in the plasmid pdBPV-MMTneo (ATCC 37224) which can serve as the starting material for the construction of other expression plasmids that would also be useful in producing LP276 polypeptides.

Transfection of mammalian cells with vectors can be performed by a plurality of well-known processes including, but not limited to, protoplast fusion, calcium phosphate co-precipitation, electroporation and the like. See, e.g., Maniatis, et al., supra.

Some viruses also make appropriate vectors. Examples include the adenoviruses, the adeno-associated viruses, the vaccinia virus, the herpes viruses, the baculoviruses, and the Rous Sarcoma virus, as described in U.S. Pat. No. 4,775,624, incorporated herein by reference.

Eukaryotic microorganisms such as yeast and other fungi are also suitable host cells. The yeast Saccharomyces cerevisiae is the preferred eukaryotic microorganism. Other yeasts such as Kluyveromyces lactis and Pichia pastoris are also suitable. For expression in Saccharomyces, the plasmid YRp7 (ATCC 40053), for example, may be used. See, e.g., Stinchcomb, et al., Nature 282(5734): 39-43 (1979); Kingsman, et al., Gene 7(2): 141-52 (1979); Tschumper and Carbon, Gene 10(2): 157-66 (1980). Plasmid YRp7 contains the TRP1 gene that provides a selectable marker for use in a trp1 auxotrophic mutant.

Production of Antibodies

The methods of the present invention may also rely on use of LP-epitope-recognizing antibodies to treat various conditions relating to allergic responses, allergic autoimmune diseases, type 1 diabetes, Th1-dependent insulitis, immunodeficiencies, cancers, inflammation, or infectious diseases. The production of antibodies, including both monoclonal and polyclonal, in animals, especially mice, is well known in the art. See, e.g., Milstein, Handbook of Experimental Immunology, Blackwell Scientific Pub. (1986); Goding, Monoclonal Antibodies: Principles and Practice, Academic Press (1983). For the production of monoclonal antibodies, the basic process begins with injecting a mouse, or other suitable animal, with an immunogen. The mouse is subsequently sacrificed and cells taken from its spleen are fused with myeloma cells, resulting in a hybridoma that reproduces in vitro. The population of hybridomas is screened to isolate individual clones, each of which secretes a single antibody species, specific for the immunogen. Each antibody obtained in this way is the clonal product of a single B cell.

Chimeric antibodies are described in U.S. Pat. No. 4,816,567, the entire contents of which are herein incorporated by reference. This reference discloses methods and vectors for the preparation of chimeric antibodies. An alternative approach is provided in U.S. Pat. No. 4,816,397, the entire contents of which are herein incorporated by reference. This patent teaches co-expression of the heavy and light chains of an antibody in the same host cell.

The approach of U.S. Pat. No. 4,816,397 has been further refined in European Patent Publication 0 239 400. The teachings of this European patent publication are a preferred format for genetic engineering of monoclonal antibodies. In this technology the complementarily determining regions (CDRs) of a human antibody are replaced with the CDRs of a murine monoclonal antibody, thereby converting the specificity of the human antibody to the specificity of a murine antibody.

Single chain antibodies and libraries thereof are yet another variety of genetically engineered antibody technology that is well known in the art. (See, e.g, Bird, et al., Science 242(4877):423-6 (1988); WO 88/01649, WO 90/14430, and WO 91/10737). Single chain antibody technology involves covalently joining the binding regions of heavy and light chains to generate a single polypeptide chain. The binding specificity of the intact antibody molecule is thereby reproduced on a single polypeptide chain.

The proteins or suitable fragments thereof required to generate polyclonal or monoclonal antibodies, and various interspecies hybrids, or humanized antibodies, or antibody fragments, or single-chain antibodies are disclosed herein. The techniques for producing antibodies are well known to skilled artisans. See, e.g., Campbell, Monoclonal Antibody Technology: Laboratory Techniques in Biochemistry and Molecular Biology, Elsevier Science Publishers, Amsterdam (1984); Kohler and Milstein, Nature 256(5517):495-7 (1975); Monoclonal Antibodies: Principles & Applications, Eds. Birch and Lennox, Wiley-Liss (1995).

The most preferred method of generating MAbs to the polypeptides and glycopeptides of the present invention comprises producing said MAbs in a transgenic mammal modified in such a way that they are capable of producing fully humanized MAbs upon antigenic challenge. Humanized MAbs and methods for their production are generally known in the art (see, e.g., U.S. Pat. Nos. 4,704,362; 4,816,567; 5,434,340; 5,545,806; 5,530,101; 5,569,825; 5,585,089; 5,625,126; 5,633,425; 5,643,763; 5,693,761; 5,693,762; 5,714,350; 5,874,299; 5,877,397; 5,939,598; 6,023,010; and 6,054,297; and PCT applications WO 96/34096; WO 96/33735; and WO 98/24893).

A protein used as an immunogen may be modified or administered in an adjuvant, by subcutaneous or intraperitoneal injection into, for example, a mouse or a rabbit. For the production of monoclonal antibodies, spleen cells from immunized animals are removed, fused with myeloma cells, such as SP2/0-Ag14 cells, and allowed to become monoclonal antibody producing hybridoma cells in the manner known to the skilled artisan. Hybridomas that secrete a desired antibody molecule can be screened by a variety of well known methods, for example ELISA assay, Western blot analysis, or radioimmunoassay [Lutz, et al., Exp. Cell Res. 175(1):109-24 (1988); Monoclonal Antibodies: Principles & Applications, Eds. Birch and Lennox, Wiley-Liss (1995)].

Nucleic Acids

The synthesis of the LP polynucleotides (such as provided in SEQ ID NO:1, 3, 5, or 7) and related nucleic acids that would encode LP polypeptides as defined herein or fragments thereof is well known in the art. See, e.g., Brown, et al., Meth. Enzymol. 68:109-51 (1979). Fragments of the DNA sequence corresponding to LP sequences could be generated using a conventional DNA synthesizing apparatus, such as the Applied Biosystems Model 380A or 380B DNA synthesizers (Applied Biosystems, Inc., Foster City, Calif.) using phosphoramidite chemistry, thereafter ligating the fragments so as to reconstitute the entire LP sequence. Alternatively, phosphotriester chemistry may be employed to synthesize the nucleic acids of this invention. See, e.g., Gait, ed., Oligonucleotide Synthesis, A Practical Approach (1984).

In an alternative methodology, namely PCR, the DNA sequences disclosed and described herein, comprising, for example, a portion or all of SEQ ID NO:1, can be produced from a plurality of starting materials. For example, starting with a cDNA preparation (e.g., cDNA library) derived from a tissue that expresses the LP276 gene, suitable oligonucleotide primers complementary to regions of SEQ ID NO:1 or to any sub-region therein, are prepared as described in U.S. Pat. No. 4,889,818, hereby incorporated by reference. Other suitable protocols for the PCR are disclosed in PCR Protocols: A Guide to Method and Applications, Innis, et al., Academic Press, Inc. (1990). Using PCR, any region of the LP276 gene can be targeted for amplification such that full or partial length gene sequences containing a functional extracellular domain may be produced.

In certain embodiments, it is advantageous to use oligonucleotide primers. The sequence of such primers is designed using a polynucleotide of the present invention for use in detecting, amplifying, or mutating a defined segment of a gene or polynucleotide that encodes an LP polypeptide using PCR technology.

The ionic strength and incubation temperature under which a probe will be used should also be taken into account. It is known that hybridization will increase as the ionic strength of the reaction mixture increases, and that the thermal stability of molecular hybrids will increase with increasing ionic strength. On the other hand, chemical reagents such as formamide, urea, DMSO and alcohols, which disrupt hydrogen bonds, increase the stringency of hybridization. Destabilization of hydrogen bonds by such reagents can greatly reduce the Tm (melting temperature). In general, optimal hybridization for synthetic oligonucleotide probes of about 10-50 bases in length occurs approximately five degrees C. below the melting temperature for a given duplex. Incubation at temperatures below the optimum may allow mismatched base sequences to hybridize and can therefore result in reduced specificity.

The length of the target nucleic acid sequence and, accordingly, the length of the probe sequence can also be important. In some cases, there may be several sequences from a particular region, varying in location and length, which will yield probes with the desired hybridization characteristics. In other cases, one sequence may be significantly better than another, even though the one sequence differs merely by a single base. Finally, there can be intramolecular and intermolecular hybrids formed within a probe if there is sufficient self-complementarily. Such structures can be avoided through careful probe design. Computer programs are available to search for this type of interaction.

The present disclosure provides exemplary methods for constructing a recombinant host cell capable of expressing proteins comprising LP polypeptides, said method comprising transforming or otherwise introducing into a host cell a recombinant DNA vector that comprises an isolated DNA sequence that encodes polypeptides comprising sequences as shown in SEQ ID NO:2, 4, 6, or 8, or fragments thereof. The preferred host cell is any eukaryotic cell which can accommodate high level expression of an exogenously introduced gene or protein, and that will incorporate said protein into its membrane structure. The skilled artisan understands that choosing the most appropriate cloning vector or expression vector depends upon a number of factors including the availability of restriction enzyme sites, the type of host cell into which the vector is to be transfected or transformed, the purpose of the transfection or transformation (e.g., stable transformation as an extrachromosomal element, or integration into the host chromosome), the presence or absence of readily assayable or selectable markers (e.g., antibiotic resistance and metabolic markers of one type and another), and the number of copies of the gene desired in the host cell.

When preparing an expression vector the skilled artisan understands that there are many variables to be considered, for example, whether to use a constitutive or inducible promoter. The practitioner also understands that the amount of nucleic acid or protein to be produced dictates, in part, the selection of the expression system. Regarding promoter sequences, inducible promoters are preferred because they enable high level, regulatable expression of an operably linked gene. The skilled artisan will recognize a number of suitable promoters that respond to a variety of inducers, for example, carbon source, metal ions, and heat. Other relevant considerations regarding an expression vector include whether to include sequences for directing the localization of a recombinant protein. For example, a sequence encoding a signal peptide preceding the coding region of a gene is useful for directing the extracellular export of a resulting polypeptide. Transformed host cells may be cultured under conditions well known to skilled artisans such that a polypeptide comprising sequence as shown in SEQ ID NO:2 is expressed, thereby producing a recombinant LP276 protein in the recombinant host cell.

Transgenic and Chimeric Non-Human Mammals

Nucleic acids which encode an LP276 polypeptide of the present invention or any of its modified forms can also be used to generate either transgenic animals or “knock out” animals which, in turn, are useful in the development and screening of therapeutically useful reagents. Methods for generating transgenic animals, particularly animals such as mice or rats, have become conventional in the art and are described, for example, in U.S. Pat. Nos. 4,736,866 and 4,870,009. Typically, particular cells would be targeted for an LP transgene incorporation with tissue-specific enhancers. Transgenic animals that include a copy of a transgene introduced into the germ line of the animal at an embryonic stage can be used to examine the effect of increased expression of DNA encoding an LP polypeptide. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologs of LP polynucleotides can be used to construct a “knock out” animal which has a defective or altered gene encoding a particular LP polypeptide as a result of homologous recombination between the endogenous gene encoding the LP polypeptide and the altered genomic DNA introduced into an embryonic cell of the animal. For example, cDNA encoding an LP276 polypeptide can be used to clone genomic DNA encoding that LP276 polypeptide in accordance with established techniques. A portion of the genomic DNA encoding an LP276 polypeptide can be deleted or replaced with another gene, such as a gene encoding a selectable marker that can be used to monitor integration. Typically, several kilobases of unaltered flanking DNA (both at the 5′ and 3′ ends) are included in the vector [see, e.g., Thomas and Capecchi, Cell 51(3):503-12 (1987) for a description of homologous recombination vectors]. The vector is introduced into an embryonic stem cell line (e.g., by electroporation), and cells in which the introduced DNA has homologously recombined with the endogenous DNA are selected [see, e.g., Li, et al., Cell 69(6):915-26 (1992)]. The selected cells are then injected into a blastocyst of an animal (e.g., a mouse or rat) to form aggregation chimeras [see, e.g., Bradley, Teratocarcinomas and Embryonic Stem Cells: A Practical Approach, Robertson, ed. (IRL, Oxford, 1987), pp. 113-52]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized, for instance, for their ability to defend against certain pathological conditions and for their development of pathological conditions due to absence of the native LP276 polypeptide.

Transgenic non-human mammals are useful as animal models in both basic research and drug development endeavors. Transgenic animals expressing at least one LP polypeptide or nucleic acid can be used to test compounds or other treatment modalities which may prevent, suppress, or cure a pathology or disease associated with at least one of the above mentioned activities. Such transgenic animals can also serve as a model for the testing of diagnostic methods for those same diseases. Furthermore, tissues derived from such transgenic non-human mammals are useful as a source of cells for cell culture in efforts to develop in vitro bioassays to identify compounds that modulate LP polypeptide activity or LP polypeptide dependent signaling. Accordingly, another aspect of the present invention contemplates a method of identifying compounds efficacious in the treatment of at least one previously described disease or pathology associated with LP polypeptide associated activity. A non-limiting example of such a method comprises:

a) generating a transgenic non-human animal which expresses an LP276 polypeptide of the present invention and which is, as compared to a wild-type animal, pathologically distinct in some detectable or measurable manner from wild-type version of said non-human mammal;

b) exposing said transgenic animal to a compound, and;

c) determining the progression of the pathology in the treated transgenic animal, wherein an arrest, delay, or reversal in disease progression in transgenic animal treated with said compound as compared to the progression of the pathology in an untreated control animals is indicative that the compound is useful for the treatment of said pathology.

Another embodiment of the present invention provides a method of identifying compounds capable of inhibiting LP polypeptide activity in vivo and/or in vitro wherein said method comprises:

a) administering an experimental compound to an LP polypeptide-expressing transgenic non-human animal or tissues derived therefrom, exhibiting one or more physiological or pathological conditions attributable to the expression of an LP transgene; and

b) observing or assaying said animal and/or animal tissues to detect changes in said physiological or pathological condition or conditions.

Another embodiment of the invention provides a method for identifying compounds capable of overcoming deficiencies in LP polypeptide activity in vivo or in vitro wherein said method comprises:

a) administering an experimental compound to an LP polypeptide-expressing transgenic non-human animal, or tissues derived therefrom, exhibiting one or more physiological or pathological conditions attributable to the disruption of the endogenous LP polypeptide-encoding gene; and

b) observing or assaying said animal and/or animal tissues to detect changes in said physiological or pathological condition or conditions.

Various means for determining a compound's ability to modulate the activity of an LP polypeptide in the body of the transgenic animal are consistent with the invention. Observing the reversal of a pathological condition in the LP polypeptide expressing transgenic animal after administering a compound is one such means. Another more preferred means is to assay for markers of LP activity in the blood of a transgenic animal before and after administering an experimental compound to the animal. The level of skill of an artisan in the relevant arts readily provides the practitioner with numerous methods for assaying physiological changes related to therapeutic modulation of LP activity.

“Gene therapy” includes both conventional gene therapies, where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has been shown that short antisense oligonucleotides can be imported into cells where they act as inhibitors, despite their low intracellular concentrations caused by their restricted uptake by the cell membrane [Zamecnik, et al., Proc. Natl. Acad Sci. USA 83(12):4143-6 (1986)]. The oligonucleotides can be modified to enhance their uptake, e.g., by substituting their negatively charged phosphodiester groups with uncharged groups.

There are a variety of techniques available for introducing nucleic acids into viable cells. The techniques vary depending upon whether the nucleic acid is transferred into cultured cell in vitro or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically, retroviral) vectors and viral coat protein-liposome mediated transfection [Dzau, et al., Trends in Biotechnology 11(5):205-10 (1993)]. In some situations it is desirable to provide the nucleic acid source with an agent that targets the target cells, such as an antibody specific for a cell surface membrane protein or the target cell, a ligand for a receptor on the target cells, etc. Where liposomes are employed, proteins which bind to a cell surface membrane protein associated with endocytosis may by used for targeting and/or to facilitate uptake, e.g., capsid proteins or fragments thereof trophic for a particular cell type, antibodies for proteins which undergo internalization in cycling, proteins that target intracellular localization and enhance intracellular half-life. The technique of receptor-mediated endocytosis is described, for example by Wu, et al., J. Biol. Chem. 262(10):4429-32 (1987); and Wagner, et al., Proc. Natl. Acad. Sci. USA 87(9):3410-4 (1990). For a review of gene marking and gene therapy protocols, see Anderson, Science 256(5058):808-13 (1992).

Methods of Treatment using LP Polypeptides

Data presented in Example 11 show that sepsis, grain-negative bacteremia, acute inflammation, and conditions or symptoms related thereto may be treated or prevented by administration of effective amounts of LP polypeptides. Administration of LP276ATFV inhibited the effects occurring during acute endotoxic shock and prevented death. As characterized generally, the invention also relates to methods preventing or treating conditions caused or exacerbated by chronic inflammation including, but not limited to, allergic responses, allergic autoimmune diseases, type 1 diabetes, Th1-dependent insulitis, inflammation, multiple sclerosis, rheumatoid arthritis, inflammatory bowel disease, liver failure, ARDS, and conditions or symptoms related thereto by administering.

Substantially pure or purified preparations of LP polypeptides can be formulated into a pharmaceutically acceptable composition. Such formulations can be dosed in a fashion consistent with good medical practice, taking into account the clinical condition of the individual patient (especially the side effects of treatment with LP polypeptides alone), the site of delivery of the LP polypeptide compositions, the method of administration, the scheduling of administration, and other factors known to practitioners.

An effective amount of an LP polypeptide will serve to prevent or treat at least one symptom of allergic responses, allergic autoimmune diseases, type 1 diabetes, Th1-dependent insulitis, immunodeficiencies, cancers, inflammation, or infectious diseases, or will serve to modulate the biological activity of at least one natural ligand. An effective amount of an LP polypeptide to prevent or treat at least one symptom may be determined by prevention or amelioration of adverse conditions or symptoms of the diseases being treated. The therapeutically effective amount of an LP polypeptide for purposes herein is thus determined by such considerations. By delivery of graduating levels of LP polypeptide within a pharmaceutical composition, a clinician skilled in the art can determine the therapeutically effective dose of an LP polypeptide for treatment or prevention of sepsis, gram-negative bacteremia, allergic responses, allergic autoimmune diseases, type 1 diabetes, Th1-dependent insulitis, immunodeficiencies, cancers, inflammation, or infectious diseases. Such determinations are well known in the art and within the skill of the clinician in adjusting the therapeutically effective amount of an LP polypeptide in a pharmaceutical composition accordingly. A therapeutically effective amount of an LP polypeptide results in a measurable modulation of the biological activity associated with an LP polypeptide.

As a general proposition, the total therapeutically effective amount of an LP polypeptide administered parentally per dose of a pharmaceutical composition will be in the range of about 1 μg/kg/day to 10 mg/kg/day of patient body weight. However, as noted above, this will be subject to therapeutic discretion. Preferably, this dose is at least 0.001 mg/kg/day, or at least 0.01 mg/kg/day, or at least 0.10 mg/kg/day, or at least 1.0 mg/kg/day.

As a further proposition, if given continuously, an LP polypeptide is typically administered at a dose rate of about 0.1 μg/kg/hour to about 50 μg/kg/hour, either by one to four injections per day or by continuous subcutaneous infusions, for example, using a mini-pump. An intravenous bag solution may also be employed. The length of treatment needed to observe changes and the interval following treatment for responses to occur appear to vary depending on the desired effect.

Pharmaceutical compositions containing an LP polypeptide may be administered using a variety of modes that include, but are not limited to, oral, rectal, intra-cranial, parenteral, intracisternal, intrathecal, intravaginal, intraperitoneal, intratracheal, intrabroncho-pulmonary, topical, transdermal (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include, but are not limited to, intravenous, intramuscular, intraperitoneal, intrasternal, subcutaneous and intraarticular injection and infusion. Implants comprising an LP polypeptide also can be used.

LP polypeptides are also suitably administered by sustained-release systems. Suitable examples of sustained-release compositions include semi-permeable polymer matrices, e.g., films, or microcapsules. Sustained-release matrices include polylactides (U.S. Pat. No. 3,773,919, EP 058 481), copolymers of L-glutamic acid and gamma-ethyl-L-glutamate [Sidman, et al., Biopolymers 22:547-56 (1983)], poly-(2-hydroxyethyl-methacrylate) [Langer, et al., J. Biomed. Matl. Res. 15:167-277 (1981)], ethylene vinyl acetate (Langer, et al., 1982) or poly-D-3-hydroxybutyric acid (EP 133 988).

Sustained-release LP polypeptide compositions also include liposomally entrapped LP276 polypeptides. Liposomes containing LP polypeptides are prepared by methods known per se [DE 3 218 121; Epstein, et al., Proc. Natl. Acad. Sci. USA 82:3688-92 (1985); Hwang, et al., Proc. Natl. Acad. Sci. USA 77:4030-4 (1980); EP 52 322; EP 36 676; EP 88 046; EP 143 949; EP 142 641; Japanese Patent Application 83-118008; U.S. Pat. Nos. 4,485,045 and 4,544,545; and EP 102 324]. Ordinarily, the liposomes are of the small (about 200 to 800 Angstroms) unilamellar type in which the lipid content is greater than about 30 mol percent cholesterol, the selected proportion being adjusted for the optimal LP polypeptide therapy.

For parenteral administration, in one embodiment, an LP polypeptide is formulated generally by mixing at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting an LP polypeptide uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

An LP polypeptide is typically formulated in such vehicles at a concentration of about 0.1 mg/mL to 100 mg/mL, preferably 1 to 10 mg/mL, at a pH of about three to eight. It will be understood that the use of certain of the foregoing excipients, carriers, or stabilizers will result in the formation of LP polypeptide salts. Pharmaceutical compositions comprising LP polypeptides to be used for therapeutic administration must be sterile. Sterility is readily accomplished by filtration through sterile filtration membranes (e.g., 0.2 micron membranes). Pharmaceutical compositions comprising LP polypeptides generally are placed into a container having a sterile access port, for example, an intravenous solution bag or vial having a stopper pierceable by a hypodermic injection needle.

Pharmaceutical compositions comprising LP polypeptides ordinarily will be stored in unit or multi-dose containers, for example, sealed ampoules or vials, as an aqueous solution or as a lyophilized formulation for reconstitution. As an example of a lyophilized formulation, ten-milliliter vials are filled with five milliliters sterile-filtered 1% (w/v) aqueous LP polypeptide solution, and the resulting mixture is lyophilized. The infusion solution is prepared by reconstituting the lyophilized LP polypeptide using bacteriostatic water-for-injection.

The present invention includes methods for the treatment or prevention of sepsis, gram negative bacteremia, allergic responses, allergic autoimmune diseases, type 1 diabetes, Th1-dependent insulitis, immunodeficiencies, cancers, inflammation, infectious diseases, and conditions or symptoms related thereto, comprising administering pharmaceutical compositions comprising LP polypeptides to a patient in need of such therapy wherein said composition further comprises other therapeutic compounds.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present application, including definitions, will control. In addition, the materials, methods and examples described herein are illustrative only and not intended to be limiting.

The following examples more fully describe the present invention.

### EXAMPLES

### Example 1

### Northern Blot and RT-PCR Analysis of LP Expression

Northern blot analysis is carried out to examine LP gene expression in human tissues, using methods described in Ausubel, et al., ed., Current Protocols in Molecular Biology, John Wiley and Sons, NY (1987-1999). A cDNA probe containing the entire nucleotide sequence of LP276 polypeptide is labeled with ³²P using the Random Prime™ DNA labeling system (Amersham Life Science), according to manufacturer's instructions. After labeling, the probe is purified using a CHROMA SPIN-100™ column (Clontech Laboratories, Inc.), according to manufacturer's protocol number PT1200-1. The purified labeled probe is then used to examine various human tissues for LP276 mRNA.

Multiple Tissue Northern (MTN) blots containing various human tissues (H) or human immune system tissues (IM) are obtained from Clontech and are examined with the labeled probe using ExpressHyb hybridization solution (Clontech) according to manufacturer's protocol number PT1190-1. Following hybridization and washing, the blots are mounted and exposed to film at negative 70 degrees C. overnight, and films developed according to standard procedures.

### Example 2

### Cloning and Expression of LP polypeptides in Mammalian Cells

A typical mammalian expression vector contains at least one promoter element, which mediates the initiation of transcription of mRNA, the polypeptide coding sequence, and signals required for the termination of transcription and polyadenylation of the transcript. Additionally, each mammalian expression vector may have enhancers, Kozak sequences and intervening sequences flanked by donor and acceptor sites for RNA splicing.

Highly efficient transcription can be achieved with the early and late promoters from SV40 and the long terminal repeats (LTRS) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pIRES1neo, pRetro-Off, pRetro-On, PLXSN, or pLNCX (Clontech Labs, Palo Alto, Calif.), pcDNA3.1 (+/−), pcDNA/Zeo (+/−) or pcDNA3.1/Hygro (+/−) (Invitrogen), PSVL and PMSG (Pharmacia, Uppsala, Sweden), pRSVcat (ATCC 37152), pSV2dhfr (ATCC 37146) and pBC12MI (ATCC 67109). Mammalian host cells that could be used include human HeLa, 293, H9 and Jurkat cells, mouse NIH3T3 and C127 cells, Cos 1, Cos 7 and CV 1, quail QC1-3 cells, mouse L cells and Chinese hamster ovary (CHO) cells.

Alternatively, the desired LP-encoding DNA sequences can be expressed in stable cell lines that contain the DNA sequences for expressing each subunit integrated into a chromosome(s). The co-transfection with a selectable marker such as DHFR (dihydrofolate reductase), gpt, neomycin, or hygromycin allows the identification and isolation of the transfected cells as known in the art.

The transfected LP polypeptide-encoding DNA sequences can also be amplified to express large amounts of the encoded polypeptide. The DHFR marker is useful to develop cell lines that carry several hundred or even several thousand copies of the DNA sequence of interest. Another useful selection marker is the enzyme glutamine synthase (GS) [Murphy, et al., Biochem. J. 227:277-9 (1991); Bebbington, et al., Bio/Technology 10:169-75 (1992)]. Using these markers, the mammalian cells are grown in selective medium, and the cells with the highest resistance are selected. These cell lines contain the amplified gene(s) integrated into a chromosome. Chinese hamster ovary (CHO) and NSO cells are often used for the production of proteins and polypeptides.

The expression vectors pC1 and pC4 contain the strong promoter (LTR) of the Rous Sarcoma virus [Cullen, et al., Mol. Cell. Biol. 5:438-47 (1985)] plus a fragment of the CMV-enhancer [Boshart, et al., Cell 41:521-30 (1985)]. Multiple cloning sites, e.g., with the restriction enzyme cleavage sites BamHI, XbaI and Asp718, facilitate the cloning of the DNA sequences of interest. The vectors contain, in addition to the 3′ intron, the polyadenylation and termination signal of the rat preproinsulin gene.

293T cells can be transfected with a PvuI linearized expression plasmid using the calcium phosphate co-precipitation method. Neomycin clones can be selected in 400 μg/mL G418 and selected clones expanded. Producing clones can be selected using an enzyme-linked immunosorbent assay with anti-human IgG1 and Northern analysis with ³²P-labeled LP-specific DNA probe. Similarly, clones producing the LP-Fc product can be produced in COS or CHO cells.

### Example 2(a)

### Cloning and Expression of LP Polypeptides in COS Cells

A plasmid for expressing LP polypeptides is made by cloning a cDNA encoding LP polypeptides into the expression vector pcDNAI/Amp or pcDNAIII (Invitrogen, Inc.). The expression vectors pcDNAI/amp and pcDNA III contain: (1) an E. coli origin of replication effective for propagation in E. coli and other prokaryotic cells; (2) an ampicillin resistance gene for selection of plasmid-containing prokaryotic cells; (3) an SV40 origin of replication for propagation in eukaryotic cells; (4) a CMV promoter, a polylinker, an SV40 intron; (5) several codons encoding a hemagglutinin fragment (i.e., an “HA” tag to facilitate purification) or HIS tag [see, e.g., Ausubel, et al., ed., Current Protocols in Molecular Biology, John Wiley and Sons, NY (1987-1999)] followed by a termination codon and polyadenylation signal arranged so that a cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin polypeptide as has been previously described [Wilson, et al., Cell 37(3):767-78 (1984)]. The fusion of the HA tag to LP polypeptide allows easy detection and recovery of the recombinant polypeptide with an antibody that recognizes the HA epitope. pcDNAIII contains, in addition, the selectable neomycin marker.

A DNA fragment encoding an LP polypeptide is separately cloned into the polylinker region of the vector so that recombinant polypeptide expression is directed by the CMV promoter. Insertion into the vector is optionally with or without the HA epitope. The plasmid construction strategy is as follows. An LP polypeptide-encoding DNA can be amplified using primers that contain convenient restriction sites. The PCR amplified LP-encoding DNA fragment and the pcDNAI/Amp vector are digested with suitable restriction enzyme(s), and the LP-encoding DNA fragment is ligated to a digested vector. Each ligation mixture is transformed into E. coli strain SURE (available from Stratagene Cloning Systems, La Jolla, Calif.), and the transformed culture is plated on ampicillin media plates which then are incubated to allow growth of ampicillin-resistant colonies. Plasmid DNA for each subunit is isolated from resistant colonies and examined by restriction analysis or other means for the presence of LP-encoding fragment.

For expression of LP polypeptides, COS cells are co-transfected with an expression vector, as described above, using DEAE-DEXTRAN, as described, for instance, in Sambrook, et al., Molecular Cloning: A Laboratory Manual, Cold Spring Laboratory Press, Cold Spring Harbor, New York (1989). Cells are incubated under conditions suitable for expression of LP polypeptide.

The LP-HA fusion polypeptide is detected by radiolabeling and immunoprecipitation, using methods described in, for example, Harlow, et al., Antibodies: A Laboratory Manual, 2nd Ed., Cold Spring Harbor Laboratory Press, Cold Spring Harbor, N.Y. (1988). To this end, two days after transfection, the cells are labeled by incubation in media containing ³⁵S-cysteine for eight hours. The cells and the media are collected, and the cells are washed and lysed with detergent-containing RIPA buffer: 150 mM sodium chloride, 1% NP-40, 0.1% SDS, 0.5% DOC, 50 mM TRIS, pH 7.5, as described by Wilson, et al., Cell 37(3):767-78 (1984). Proteins are precipitated from the cell lysate and from the culture media using an HA-specific monoclonal antibody. The precipitated protein is then analyzed by SDS-PAGE and autoradiography. An expression product of the expected size is seen in the cell lysate, which is not seen in negative controls.

### Example 2(b)

### Cloning and Expression of LP Polypeptides in CHO Cells

The vector pC4 can be used for expression of LP polypeptide. Plasmid pC4 is a derivative of the plasmid pSV2-dhfr (ATCC 37146). The plasmid contains the mouse DHFR gene under control of the SV40 early promoter. Chinese hamster ovary (dhfr-) or other cells lacking dihydrofolate activity that are co-transfected with LP plasmids can be selected by growing the cells in a selective medium (alpha minus MEM, Life Technologies) supplemented with the chemotherapeutic agent methotrexate (MTX). The amplification of the DHFR genes in cells resistant to methotrexate has been well documented [see, e.g., Alt, et al., J. Biol. Chem. 253:1357-70 (1978); Hamlin and Ma, Biochem. et Biophys. Acta 1097:107-43 (1990); and Page and Sydenham, Biotechnology 9:64-8 (1991)]. Cells grown in increasing concentrations of MTX develop resistance to the drug by overproducing the target enzyme, DHFR, as a result of amplification of the DHFR gene. If DNA sequences are linked to the DHFR gene, they are usually co-amplified and over-expressed. It is known in the art that this approach can be used to develop cell lines carrying more than one thousand copies of the amplified gene(s). Subsequently, when the methotrexate is withdrawn, cell lines are obtained which contain the amplified DNA sequences integrated into one or more chromosome(s) of the host cell.

Plasmid pC4 contains the strong promoter of the long terminal repeat (LTR) of the Rous Sarcoma virus [Cullen, et al., Mol. Cell. Biol. 5:438-47 (1985)] for expression of inserted gene sequences. PC4 additionally contains a fragment isolated from the enhancer of the immediate early gene of human cytomegalovirus (CMV) [Boshart, et al., Cell 41:521-30 (1985)]. Downstream of the promoter are BamHI, XbaI, and Asp718 restriction enzyme cleavage sites that allow integration of the DNA sequences. Behind these cloning sites the plasmid contains the 3′ intron and polyadenylation site of the rat preproinsulin gene. Other high efficiency promoters can also be used for the expression, e.g., the human beta-actin promoter, the SV40 early or late promoters, or the long terminal repeats from other retroviruses, e.g., HIV and HTLVI. Clontech's Tet-Off and Tet-On gene expression systems and similar systems can be used to express LP polypeptide in a regulated way in mammalian cells [Gossen and Bujard, Proc. Natl. Acad. Sci. USA, 89:5547-51 (1982)]. For the polyadenylation of the mRNA, other signals, e.g., from the human growth hormone or globin genes, can be used as well. Stable cell lines carrying the DNA sequences of interest integrated into the chromosomes can also be selected upon co-transfection with a selectable marker such as gpt, G418, or hygromycin. It is advantageous to use more than one selectable marker in the beginning, e.g., G418 plus methotrexate.

The plasmid pC4 is digested with restriction enzymes and then dephosphorylated using calf intestinal phosphatase by procedures known in the art. The vector is then isolated from a 1% agarose gel.

The DNA sequence encoding the complete LP polypeptide is amplified using PCR oligonucleotide primers corresponding to the 5′ and 3′ sequences of the gene. Non-limiting examples include 5′ and 3′ primers having nucleotides corresponding or complementary to a portion of the coding LP sequences according to methods known in the art.

The amplified fragment(s) is digested with suitable endonucleases and then purified again on a 1% agarose gel. The isolated fragment for each subunit and the dephosphorylated vector are then separately ligated with T4 DNA ligase. E. coli HB101 or XL-1 Blue cells are separately transformed, and bacteria are identified that contain the fragment (or fragments, if the vector is adapted for expressing both alpha and beta subunits) inserted into plasmid pC4 using, for instance, restriction enzyme analysis.

Chinese hamster ovary (CHO) cells lacking an active DHFR gene are used for transfection. Five micrograms of the expression plasmid(s) pC4 is cotransfected with 0.5 μg of the plasmid pSV2-neo using lipofectin. The plasmid pSV2-neo contains a dominant selectable marker, the neo gene from Tn5 encoding an enzyme that confers resistance to a group of antibiotics including G418. The cells are seeded in alpha minus MEM supplemented with 1 μg/mL G418. After two days, the cells are trypsinized and seeded in hybridoma cloning plates (Greiner, Germany) in alpha minus MEM supplemented with 10, 25, or 50 ng/mL of methotrexate plus 1 μg/mL G418. After about ten to fourteen days, single clones are trypsinized and then seeded in six-well petri dishes or ten milliter flasks using different concentrations of methotrexate (50 nM, 100 nM, 200 nM, 400 nM, and 800 nM). Clones growing at the highest concentrations of methotrexate are then transferred to new six-well plates containing even higher concentrations of methotrexate (1 mM, 2 mM, 5 mM, 10 mM, and 20 mM). The same procedure is repeated until clones are obtained which grow at a concentration of 100 to 200 mM. Expression of the desired product is analyzed, for instance, by SDS-PAGE and Western blot or by reversed-phase HPLC analysis.

### Example 3

### Prokaryotic Expression and Purification of LP Protein

The bacterial expression vector pQE60 is used for bacterial expression in this example. (QIAGEN, Inc., Chatsworth, Calif.). pQE60 encodes ampicillin antibiotic resistance (“Ampr”) and contains a bacterial origin of replication (“ori”), an IPTG inducible promoter, a ribosome binding site (“RBS”), six codons encoding histidine residues that allow affinity purification using nickel-nitrilo-tri-acetic acid (“Ni-NTA”) affinity resin sold by QIAGEN, Inc., and suitable single restriction enzyme cleavage sites.

These elements are arranged such that a DNA fragment encoding a polypeptide can be inserted in such a way as to produce that polypeptide with the six His residues (i.e., a “6×His tag”) covalently linked to the carboxyl terminus of that polypeptide. However, a polypeptide coding sequence can optionally be inserted such that translation of the six His codons is prevented and, therefore, a polypeptide is produced with no 6×His tag.

The nucleic acid sequence encoding the desired portion of an LP polypeptide lacking the hydrophobic leader sequence is amplified from a cDNA clone using PCR oligonucleotide primers (based on the sequences presented, e.g., as in SEQ ID NO:1), which anneal to the amino terminal encoding DNA sequences of the desired portion of LP polynucleotide and to sequences in the construct 3′ to the cDNA coding sequence. Additional nucleotides containing restriction sites to facilitate cloning in the pQE60 vector are added to the 5′ and 3′ sequences, respectively.

For cloning LP polynucleotides, the 5′ and 3′ primers have nucleotides corresponding or complementary to a portion of the coding sequence of the LP polynucleotide, e.g., as presented in the coding regions of SEQ ID NO:1 or 3, according to known method steps. One of ordinary skill in the art would appreciate, of course, that the point in a polypeptide coding sequence where the 5′ primer begins can be varied to amplify a desired portion of the complete polypeptide shorter or longer than the mature form.

The amplified nucleic acid fragments and the vector pQE60 are digested with appropriate restriction enzymes and the digested DNAs are then ligated together. Insertion of the LP DNA into the restricted pQE60 vector places the LP polypeptide-coding region, including its associated stop codon, downstream from the IPTG-inducible promoter and in-frame with an initiating AUG codon. The associated stop codon prevents translation of the six histidine codons downstream of the insertion point.

The ligation mixture is transformed into competent E. coli cells using standard procedures such as those described in Sambrook, et al., 1989; Ausubel, 1987-1998. E. coli strain M15/rep4, containing multiple copies of the plasmid pREP4, which expresses the lac repressor and confers kanamycin resistance (“Kanr”), is used in carrying out the illustrative example described herein. This strain, which is only one of many that are suitable for expressing LP polypeptide, is available commercially from QIAGEN, Inc. Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Clones containing the desired constructs are grown overnight (“O/N”) in liquid culture in LB media supplemented with both ampicillin (100 μg/mL) and kanamycin (25 μg/mL). The O/N culture is used to inoculate a large culture, at a dilution of approximately 1:25 to 1:250. The cells are grown to an optical density at 600 nm (“OD600”) of between 0.4 and 0.6. Isopropyl-b-D-thiogalactopyranoside (“IPTG”) is then added to a final concentration of 1 mM to induce transcription from the lac repressor sensitive promoter, by inactivating the lacI repressor. Cells subsequently are incubated further for three to four hours. Cells then are harvested by centrifugation.

The cells are then stirred for three to four hours at 4 degrees C. in 6 M guanidine hydrochloride, pH 8. The cell debris is removed by centrifugation, and the supernatant containing LP polypeptide is dialyzed against 50 mM sodium acetate buffer pH 6, supplemented with 200 mM sodium chloride. Alternatively, a polypeptide can be successfully refolded by dialyzing it against 500 mM sodium chloride, 20% glycerol, 25 mM Tris hydrochloride pH 7.4, containing protease inhibitors.

If insoluble protein is generated, the protein is made soluble according to known method steps. After renaturation, the polypeptide is purified by ion exchange, hydrophobic interaction, and/or size exclusion chromatography. Alternatively, an affinity chromatography step such as an antibody column is used to obtain pure LP protein. The purified polypeptide is stored at 4 degrees C. or frozen at negative 40 degrees C. to negative 120 degrees C.

### Example 4

### Cloning and Expression of LP Polypeptide in a Baculovirus Expression System

In this example, the plasmid shuttle vector pA2 GP is used to insert the cloned DNA encoding the LP polypeptide into a baculovirus for expression using a baculovirus leader and standard methods as described in Summers, et al., A Manual of Methods for Baculovirus Vectors and Insect Cell Culture Procedures, Texas Agricultural Experimental Station Bulletin No. 1555 (1987). This expression vector contains the strong polyhedrin promoter of the Autographa californica nuclear polyhedrosis virus (AcMNPV) followed by the secretory signal peptide (leader) of the baculovirus gp67 polypeptide and convenient restriction sites such as BamHI, Xba I, and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate viable virus that expresses the cloned polynucleotide.

Other baculovirus vectors are used in place of the vector above, such as pAc373, pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow, et al., Virology 170: 31-9.

The cDNA sequence encoding the mature LP polypeptide in a clone, lacking the AUG initiation codon and the naturally associated nucleotide-binding site, is amplified using PCR oligonucleotide primers corresponding to the 5′ and 3′ sequences of the gene. Non-limiting examples include 5′ and 3′ primers having nucleotides corresponding or complementary to a portion of the coding sequence of an LP polypeptide, e.g., as presented in SEQ ID NO:2 or 4, according to known method steps.

The amplified fragment is isolated from a 1% agarose gel using a commercially available kit (e.g., “Geneclean,” BIO 101 Inc., La Jolla, Calif.). The fragment then is then digested with the appropriate restriction enzyme and again is purified on a 1% agarose gel. This fragment is designated herein “F1.”

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.). This vector DNA is designated herein “V1.”

Fragment F1 and the dephosphorylated plasmid V1 are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria are identified that contain the plasmid bearing the human LP gene using the PCR method, in which one of the primers that is used to amplify the gene and the second primer is from well within the vector so that only those bacterial colonies containing the LP gene fragment will show amplification of the DNA. The sequence of the cloned fragment is confirmed by DNA sequencing. This plasmid is designated herein pBacLP.

Five μg of the plasmid pBacLP is co-transfected with 1.0 μg of a commercially available linearized baculovirus DNA (“BaculoGold® baculovirus DNA”, PharMingen, San Diego, Calif.), using the lipofection method described by Felgner, et al., Proc. Natl. Acad. Sci. USA 84: 7413-7 (1987). One microgram of BaculoGold® virus DNA and 5 μg of the plasmid pBacLP are mixed in a sterile well of a microtiter plate containing 50 μL of serum-free Grace's medium (Life Technologies, Inc., Rockville, Md.). Afterwards, 10 μL Lipofectin plus 90 μL Grace's medium are added, mixed and incubated for fifteen minutes at room temperature. Then, the transfection mixture is added drop-wise to Sf9 insect cells (ATCC CRL 1711) seeded in a 35 mm tissue culture plate with 1 mL Grace's medium without serum. The plate is rocked back and forth to mix the newly added solution. The plate is then incubated for five hours at 27 degrees C. After five hours, the transfection solution is removed from the plate, and 1 mL of Grace's insect medium supplemented with 10% fetal calf serum is added. The plate is put back into an incubator and cultivation is continued at 27 degrees C. for four days.

After four days, the supernatant is collected, and a plaque assay is performed. An agarose gel with “Blue Gal” (Life Technologies, Inc., Rockville, Md.) is used to allow easy identification and isolation of gal-expressing clones, which produce blue-stained plaques. (A detailed description of a “plaque assay” of this type can also be found in the user's guide for insect cell culture and baculovirology distributed by Life Technologies, Inc., Rockville, Md., pages 9-10). After appropriate incubation, blue stained plaques are picked with a micropipettor tip (e.g., Eppendorf). The agar containing the recombinant viruses is then resuspended in a microcentrifuge tube containing 200 μL of Grace's medium and the suspension containing the recombinant baculovirus is used to infect Sf9 cells seeded in 35 mm dishes. Four days later, the supernatants of these culture dishes are harvested and then stored at 4 degrees C. The recombinant virus is called V-LP.

To verify the expression of LP polypeptide, Sf9 cells are grown in Grace's medium supplemented with 10% heat-inactivated FBS. The cells are infected with the recombinant baculovirus V-LP at a multiplicity of infection (“MOI”) of about two. Six hours later, the medium is removed and replaced with SF900 II medium minus methionine and cysteine (available, e.g., from Life Technologies, Inc., Rockville, Md.). If radiolabeled polypeptides are desired, 42 hours later, 5 mCi of ³⁵S-methionine and 5 mCi ³⁵S-cysteine (available from Amersham, Piscataway, N.J.) are added. The cells are further incubated for sixteen hours and then harvested by centrifugation. The polypeptides in the supernatant as well as the intracellular polypeptides are analyzed by SDS-PAGE, followed by autoradiography (if radiolabeled). Microsequencing of the amino acid sequence of the amino terminus of purified polypeptide can be used to determine the amino terminal sequence of the mature polypeptide and, thus, the cleavage point and length of the secretory signal peptide.

### Example 5

### Production of an Antibody to LP Polypeptides or Fragments Thereof

A substantially pure LP276 polypeptide or fragment thereof is isolated from transfected or transformed cells using any of the methods well known in the art or by a method specifically disclosed herein. Concentration of protein in a final preparation is adjusted, for example, by filtration through an Amicon filter device such that the level is about 1 to 5 μg/mL. Monoclonal or polyclonal antibody can be prepared as follows.

Monoclonal antibodies can be prepared from murine hybridomas according to the method of Kohler and Milstein [Nature 256(5517):495-7 (1975)] or a modified method thereof. Briefly, a mouse is repetitively inoculated using a few micrograms of the peptide, polypeptide, or fusion polypeptide, over a period of a few weeks. The mouse is then sacrificed, and the antibody producing cells of the spleen isolated. The spleen cells are fused by means of polyethylene glycol with mouse myeloma cells. Fused cells that produce antibody are identified by any suitable immunoassay, for example, ELISA, as described in Engvall, Meth. Enzymol. 70(A):419-23 (1980).

Polyclonal antiserum can be prepared by well-known methods [e.g., Vaitukaitis, et al., J. Clin. Endocrinol. Metab. 33(6):988-91 (1971)] that involve immunizing suitable animals with one or more of the LP276 polypeptides, or fragments thereof, disclosed herein. Small doses (e.g., nanograms) of antigen administered at multiple intradermal sites appear to be the most reliable method.

### Example 6

### Construction of an LP-Flag Expression Vector

To facilitate confirmation of LP polypeptide expression (without the use of LP epitope-recognizing antibodies), a bicistronic expression vector (pIG1-LPF) is constructed by insertion of an “internal ribosome entry site”/enhanced green fluorescent protein (IRES/eGFP) PCR fragment into the mammalian expression vector pGTD [Gerlitz, et al., Biochem. J. 295(Part 1):131-40 (1993)]. This new vector, designated pIG1, contains the following sequence landmarks: the E1a-responsive GBMT promoter [Berg, et al., Biotechniques 14(6):972-8 (1993); Berg, et al., Nucleic Acids Res. 20(20):5485-6 (1992)]; a unique BclI cDNA cloning site; the IRES sequence from encephalomyocarditis virus (EMCV); the eGFP (Clontech) coding sequence [Cormack, et al. Gene 173(1 Spec No):33-8 (1996)]; the SV40 small “t” antigen splice site/poly-adenylation sequences; the SV40 early promoter and origin of replication; the murine dihydrofolate reductase (dhfr) coding sequence; and the pBR322 ampicillin resistance marker/origin of replication.

A pair of primers containing the DNA sequence cleaved by the restriction enzyme BclI at their 5′ termini are synthesized so that when used to amplify the LP polypeptide encoding DNA they incorporate the DNA sequence encoding the eight amino acid Flag epitope in-frame with the DNA sequences encoding the LP polypeptide at the 3′ terminus of the amplified product [Miceli, et al., J. Immunol. Methods 167(1-2):279-87 (1994)]. These primers are used to PCR amplify the LP polypeptide-encoding DNA. The resultant PCR product (LPF) is then digested with BclI (restriction sites incorporated into the primers) and ligated into the unique BclI site of pIG1 to generate the plasmid pIG1-LPF. The human LP cDNA orientation and nucleotide sequence is confirmed by restriction digest and double stranded sequencing of the insert.

### Example 7

### Construction of a Non-Flag LP Expression Vector

In order to generate a non-Flagged expression vector (pIG1-LP), the 24-base DNA sequence encoding the eight amino acid FLAG epitope is deleted from the pIG1-LP construct using the Quik Change mutagenesis kit (Stratagene). A 35-base primer, and its complement, with identity to the 19-base sequences flanking the FLAG sequence is synthesized and used to prime PCR using the plasmid as template. The PCR product is digested with Dpn1 restriction endonuclease to eliminate the parental DNA, and the digested product is transformed into Epicurean XLI-Blue E. coli cells. Ampicillin-resistant transformants are picked and the plasmid DNA is analyzed by restriction digestion. Precise deletion of the 24-base sequence is confirmed by DNA sequencing of pIG1-LP.

### Example 8

### Construction of LP-Immunoglobulin Fusion Proteins

A. Expression of an LP-Fc Fusion

LP276 polypeptide or a fragment thereof is prepared as a fusion protein coupled to an immunoglobulin constant region. The immunoglobulin constant region may contain genetic modifications including those that reduce or eliminate effector activity inherent in the immunoglobulin structure. (See, e.g., PCT Publication WO 88/07089, published Sep. 22, 1988). Briefly, PCR overlap extension is applied to join DNA encoding the LP276 polypeptide or a fragment thereof to DNA encoding the hinge, CH2, and CH3 regions of human IgG1. This is accomplished as described in the following subsections.

A DNA fragment corresponding to the DNA sequences encoding full-length or a fragment of LP276 is prepared by polymerase chain reaction (PCR) using primer pairs designed to amplify sequences encoding LP276, having a DNA sequence 5′ to the ATG that is added to incorporate a HindIII site and an EcoRV site. A cDNA encoding full-length LP276 polypeptide serves as the template for amplifying LP276. PCR amplification with these primers generates a DNA fragment that encodes a full-length or fragment of LP276. The sequence of human IgG1 is obtained through Genbank [accession: HUMIGCC4; Takahashi, et al., Cell 29(2):671-9 (1982)]. This is compiled into exons and a region upstream of the natural hinge region chosen as the fusion site. The 5′ primer is designed to include an overlap for the LP amplicon. The 3′ primer is complementary to the Fc region and incorporates the translation stop codon.

PCR reactions are prepared in one hundred microliters final volume composed of Pfu polymerase and buffer (Stratagene) containing primers (1 μM each), dNTPs (200 μM each), and one nanogram of template DNA.

The resulting fragment is then cleaved with HindIII and EcoRV, which recognize the unique sites incorporated into the forward PCR primer and the reverse PCR primer, respectively. The digested fragment is cloned into an expression vector, XenoFLIS-Fc, that has also been treated with the same restriction enzymes.

This cloning procedure yields clones that contain the LP-Fc fusion. The sequence is confirmed by DNA sequences.

B. Isolation of Stable Clones

First, 293T cells are grown and transient transfected utilizing lipofectamine (Gibco-BRL). Characterization of the supernatant reveals a protein approximately the size of a dimer of the LP-Fc fusion, thereby confirming the integrity of the construct. The expression of the protein is confirmed by a Western blot utilizing an antibody to human IgG1.

### Example 9

### Large Scale Purification of LP Polypeptides

Large-scale production of LP polypeptides is effected by first growing stable LP-expressing clones in several ten-liter spinners. After reaching confluency, cells are further incubated for two to three more days to secrete maximum amount of LP into the media. Media containing LP polypeptide is adjusted to 0.1% CHAPS and concentrated in an Amicon ProFlux M12 tangential filtration system to 350 mL. The concentrated media is centrifuged at 19,000 rpm (43,000 g) for fifteen minutes and passed over an SP-5PW TSK-GEL column (21.5 mm×15 cm; TosoHaas) at a flow rate of 8 mL/min. The column is washed with buffer A (20 mM MOPS, 0.1% CHAPS, pH 6.5) until the absorbance (280 nm) returns to baseline, and the bound proteins are eluted with a linear gradient from 0.1 M to 0.3 M sodium chloride (in buffer A) developed over eighty-five minutes. Fractions containing the LP polypeptide are pooled and passed over a Heparin-5PW TSK-GEL column (7.5 mm×7.5 cm) equilibrated in buffer B (50 mM Tris, 0.1% CHAPS, 0.3 M sodium chloride, pH 7.0). The bound protein is eluted with a linear gradient from 0.3 M to 1.0 M sodium chloride (in buffer B) developed over sixty minutes. Fractions containing the LP polypeptide are pooled and passed over a 1 cm×15 cm Vydac C4 column equilibrated with 0.1% TFA in water. The bound LP polypeptide is eluted with a linear gradient from 0 to 100% acetonitrile with 0.1% TFA. Fractions containing the LP polypeptide are analyzed by SDS-PAGE and found to be greater than 95% pure. These fractions are dialyzed against 8 mM sodium phosphate, 0.5 M sodium chloride, 10% glycerol, pH 7.4.

### Example 10

### Production of Transgenic Mice Expressing LP Polypeptide

The LP gene fragment is excised from a DHFR vector by AscI and SalI digestion and gel-purified. This fragment is then ligated into the MluI and XhoI sites of plasmid pLIV.7 (provided by John Taylor, The J. David Gladstone Institutes) generating plasmid pLIV7-LP. Plasmid pLIV.7 is described by Fan, et al., where it is used to create plasmid pLivhHL1 [Proc. Natl. Acad. Sci. USA 91(18):8724-8 (1994)]. pLiv.7 is identical to pLivhHL 1 with the HL (hepatic lipase) sequence removed and contains the Apo E gene promoter/5′ flanking region and a hepatic enhancer sequence referred to as the hepatic control region (HCR). For microinjection into embryos, a 6.5 kb DNA fragment encompassing the Apo E gene promoter-LP48-hepatic control region (HCR) fusion gene is excised from plasmid pLIV7-LP48 by digestion with SalI and SpeI and purified by gel electrophoresis and glass bead extraction.

Transgenic mice are generated using established techniques [Hogan, et al., Manipulating the Mouse Embryo: A Laboratory Manual, Cold Spring Harbor Laboratory, NY (1986)] as modified by Fox and Solter [Mol. Cell. Biol. 8(12):5470-6 (1988)]. Briefly, the 6.5 kb DNA fragment encompassing the Apo E gene promoter-LP-HCR fusion gene Is microinjected into the male pronuclei of newly fertilized one-cell-stage embryos (zygotes) of the FVB/N strain. The embryos are cultured in vitro overnight to allow development to the two-cell-stage. Two-cell embryos are then transplanted into the oviducts of pseudopregnant CD-1 strain mice to allow development to term. To test for the presence of the transgene in the newborn mice, a small piece of toe is removed from each animal and digested with proteinase K to release the nucleic acids. A sample of the toe extract is subsequently subjected to PCR analysis using human LP-specific primers to identify transgene-containing “founder” mice. Founder transgenic mice are bred to produce stable lines of transgenics.

### Example 11

### LP Polypeptide Protects against LPS-Induced Septic Shock in Mice

This example demonstrates that LP polypeptides can protect against LPS-induced septic shock in mice. These data indicate that LP polypeptides are useful in preventing and treating such conditions.

Eight to ten-week old BALB/c mice (Harlan, Indianapolis) are given 20 μg/mouse of human LP276-Ig by intravenous injection (lateral tail vein). Twenty-four hours later, 200 μg LPS are injected intravenously into each mouse to induce sepsis. The mice are monitored three times per day for 72 hours to determine survival. LP276L polypeptide demonstrates a 100% survival rate in one experiment and 20% in another experiment.

### Example 12

### Identification of LP-Binding Proteins Including Natural LP Receptors

LP polypeptides may be used to screen for molecules that bind to LP receptors or molecules to which LP polypeptides bind. The molecules that bind to LP polypeptides may be agonists or antagonists of LP polypeptide. They may include antibodies, oligonucleotides, protein (receptor), or small molecules.

For instance, flag-tagged-LP polypeptide are incubated with cell lysates of cells suspected of expressing LP receptors in buffer containing of 10 mM Tris, 150 mM sodium chloride, 2 mM EDTA, 0.5% NP-40 and proteinase inhibitors (one pill per 50 mL buffer, Boehringer Mannheim), at 4 degrees C. for 4 hours. Anti-Flag beads (Sigma) are added, and the mixture is incubated for an additional four hours. Complexes are recovered by centrifugation, washed with twenty times bead volume of binding buffer, and eluted in fractions with tris-glycine buffer (pH 2.5). An aliquot of each fraction is separated by electrophoresis on a polyacrylamide gel. The gel is silver stained according to manufacturer's instructions (silver staining kit from Novex; San Diego, Calif.). Pools having positive bands are pooled together and concentrated. The pooled samples are again separated on a denaturing polyacrylamide gel and transferred to a PVDF membrane. Proteins that bind to LP polypeptides specifically are then identified by microsequencing, according to methods known to those skilled in the art.

### Example 13

### Use of LP Polypeptides to Treat Type I Diabetes

Female NOD/Bom mice are purchased from the Jackson Laboratory (Maine) at nine weeks of age and maintained in an animal facility under conventional conditions with standard diet. To accelerate development of diabetes, mice are treated with cyclophosphamide (250 mg/kg i.p.) at seventy days of age. One group of mice receives 50 μg/mouse/day LP polypeptide by subcutaneous injection, and another group of mice receives 50 μg/mouse/day BSA. Urinary glucose is analyzed daily, and hyperglycemia is detected by blood glucose determinations. Animals are generally regarded as diabetic when blood glucose levels are found to be above 16.7 mmol/liter as determined by hexokinase method. BSA treated mice are diagnosed with diabetes ten to eleven days after cyclophosphamide injection. Following cyclophosphoamide injection, mice are sacrificed on days one, three, six, and nine for pancreas and spleen analysis.

### Example 14

### Use of LP Polypeptides to Treat Liver Disease

It is well established that treating mice with LPS and D-galactosamine leads to clinical symptoms of severe hepatitis and shock. BALB/c mice (Harlan, Indianapolis) are divided into experimental groups of six or twelve animals, and the lateral tail vein of each animal is intravenously injected with 6 mg of D-(+)-galactosamine (Sigma, 39F-0539) in 100 μL of PBS (GibcoBRL) and 3 μg of lipopolysaccharide beta from E. Coli 026:B6 (LPS) (Difco, 3920-25-2) in 100 μL of PBS. One hour later, the animals are given intravenous injections of either (1) LP polypeptide (50 μg) or (2) BSA (50 μg) control, respectively. The survival rates of the mice variously treated mice are determined twenty-four hours after LPS injection.

### Example 15

### Use of LP276 Polypeptides to Treat Rheumatoid Arthritis (RA)

Using to the collagen-induced arthritis (CIA) model of RA, DBA/1 mice are immunized with bovine type II collagen in adjuvant and treated daily after disease onset with either (1) recombinant human LP polypeptide or (2) saline. Mice are monitored for paw swelling and clinically scored. Histology analysis is also performed. Treatment of established CIA with LP polypeptides may be effective in inhibiting paw swelling as well as disease progression as defined by clinical scoring cartilage.

### Example 16

### Use of LP Polypeptides to Treat Autoimmune Diseases Including Multiple Sclerosis

Rats are immunized by subcutaneous injection in the hind footpads with 0.1 mL of MBP epitope in PBS (1.5 mg/mL) and emulsified with an equal volume of CFA. One group of animals receives 2 mg/kg/day LP polypeptide by subcutaneous injection, and another group receives control BSA. Rats are then monitored daily for clinical signs by an observer who is blind to the treatment protocol. EAE is scored as follows: 0, clinically normal; 1, flaccid tail; 2, hind limb paralysis; and 3, front and hind limb paralysis.

### Example 17

### Use of LP Polypeptides to Treat Inflammatory Bowel Diseases

Specific pathogen-free, five to six week-old male SJL/J mice are purchased from Jackson Laboratory (Maine). Trinitrobenzene sulfonic acid (TNBS) is obtained from Sigma-Aldrich. TNBS colitis is induced as described previously [Neurath, et al., J. Exp. Med. 182(5):1281-90 (1995); Kitani, et al., J. Exp. Med. 192(l):41-52 (2000)]. In this model, 1.5 to 2.0 mg of TNBS dissolved in 50% ethanol is administrated per rectum. Seven days later, mice lose weight continuously and show other clinical features of chronic colitis. For the study of prevention of induction of TNBS colitis, LP polypeptide is administrated by subcutaneous injection at 2 mg/kg/day seven days later for one week. A control group consists of mice receiving ethanol without TNBS. The weight of each mouse is monitored every twenty-four hours, and mice are sacrificed at multiple time points for assessment of histologic findings and cytokine production.

### Example 18

### In vivo Testing of LP Polypeptide for Treatment or Prevention of ARDS

Runaway apoptosis and inflammation may lead to acute respiratory distress syndrome (ARDS) or, if multiple organs are involved, sepsis. ARDS is most often encountered with other serious illnesses. Thirty-eight percent (38%) of ARDS cases occur in sepsis patients. ARDS research efforts have focused on pro-inflammatory cytokines, specifically TNF-alpha, IL-1, IL-6, and IL-8, some of which are elevated during ARDS. Experimental treatments revolving around cytokine antagonism have included prostaglandin E1, anti-TNF, antioxidants, and antiproteases. Experimental therapies include administration of corticosteroids, ventilator therapy (PEEP), surfactant replacement therapy, and inhaled nitric oxide therapy. Unfortunately, little or no benefit has been observed clinically from any experimental treatments to date. At the present time there is no FDA-approved pharmacological treatment for ARDS.

ARDS and sepsis are characterized by an overactivation of cytokine pathways where there is massive apoptosis and/or inflammation of cells in lungs and multiple organs, respectively. Rabbits exposed to hyperoxia (100% oxygen) for sixty-four hours develop clinical symptoms that are very similar to human ARDS. For example, one molecular endpoint of the hyperoxia model is increased alveolar permeability to solute, which can be quantified. Therefore, to determine the usefulness of LP polypeptides as a prophylactic (before challenge) and/or treatment (before, during and/or after challenge), rabbits are challenged with hyperoxia to induce the ARDS symptomology, treated accordingly with LP polypeptides, followed by measurement of solute permeability across the alveolar epithelium. LP polypeptides of varying concentrations are generally given at different times. Thus, according to one embodiment of the present invention, LP polypeptides may be useful in improving lung function in sepsis and/or ARDS patients and measures of lung function may include, but are not limited to, fluid transport across the alveoli.

### Example 19

### Mixed Lymphocyte Reaction (MLR) and Cytotoxic Lymphocyte Assay (CTL)

P815 mouse mastocytoma cells are retrovirally transduced to express mouse B7.1, human LP276 or both mouse B7.1 and human LP276. Expression is confirmed by FACS staining.

B57B1/6 mice are immunized with 2×10⁶ P815 cells per injection on fourteen days and seven days before sacrificing. For the MLR assay, mice are immunized with P815 cells. For the CTL assay, mice are immunized with one of the following: P815 cells, (transduced with vector), P815 cells expressing B7.1, P815 expressing LP276, or P815 cells expressing both B7.1 and LP276.

On Day 0, mice are sacrificed and spleens harvested. A single cell suspension is made from the spleens and CD8+ T cells are negatively selected from the cell mixture. The P815 cell lines are irradiated with 5000 rads.

For the MLR, 8×10⁵ CD8+ T cells are mixed with 5×10⁴ of each of the irradiated P815 cells per well of a 96 well tissue culture plate. Plates are incubated at 37° C. with 5% carbon dioxide. On Day 4, the cells are labeled with 1 μCi/well of ³H-thymidine, incubated overnight, and harvested on Day 5.

Results for the MLR are shown in Table 11. Data indicate that P815 cells expressing B7.1 induce more proliferation than vector transduced P815 cells. P815 cells expressing both B7.1 and LP276 induce proliferation similar to levels of vector transduced P815 cell. These data suggest that the LP276 is inhibiting the B7.1 co-stimulation of proliferation.

TABLE 11
³H-thymidine incorporation on Day 4/5.

| P815 cell type | Vector | B7.1 | LP276 | LP276 and B7.1 |
|---|---|---|---|---|
| mean CPM | 11603 | 32988 | 9337 | 11915 |
| standard deviation | 1138 | 5468 | 3172 | 3499 |

For the CTL assay, the mouse CD8+ T cells are re-stimulated with irradiated P815 cells. The P815 cell type used is matched to the one used to immunize the C57B1/6 mice (i.e., the CD8+ T cells from the mice immunized with P815 cells expressing B7.1 are re-stimulated with the P815 cells expressing B7.1.)

On Day 5, these cultured cells are resuspended and put over a Ficoll gradient to remove the dead cells. The activated CD8+ T cells are washed, counted, and resuspended in media to 2×10⁷/mL. The cells are added to a 96-well, round bottom plate and two-fold dilutions are performed. This provides cells at 2×10⁶, 10⁶, 5×10⁵, 2.5×10⁵, and 1.25×10⁵. To these wells, 2×10⁴ P815 cells are added. The ratio of effector cells to target cells is 100:1, 50:1, 25:1, 12.5:1 and 6:1. The plate is centrifuged briefly to bring the cells in contact with each other and incubated at 37° C. with 5% carbon dioxide for four hours. Supernatants are harvested and the LDH release is measured using the Promega CytoTox 96® Non-Radioactive Cytotoxicity Assay (Cat. # G1780) following manufacturer's instructions.

Results for the CTL are shown in Table 12. The results show that CD8+ T cells immunized and re-stimulated with P815 expressing B7.1 have an increase in LDH release compared to CD8+ T cells immunized and re-stimulated with P815 cells transduced with the vector. These data indicate that the B7.1 is a co-stimulator of CD8+ T cell activation. The CD8+ T cells immunized with the P815 cells expressing both LP276 and B7.1 have a lower percentage of LDH, suggesting that the co-expression of LP276 inhibits the action of B7.1.

TABLE 12
Cytotoxic T Lymphocyte Assay of CD8+ T cells against P815 cells.

| | | | |
|---|---|---|---|
| | | | LP276 |
| Vector | B7.1 | LP276 | and B7.1 |

Percentage of maximum LDH release

| | | | | |
|---|---|---|---|---|
| 100:1 | 47.3 | 61.7 | 44.0 | 49.2 |
| 50:1 | 31.5 | 51.9 | 25.9 | 24.2 |
| 25:1 | 21.4 | 36.8 | 18.2 | 12.8 |
| 12.5:1 | 9.7 | 29.4 | 2.9 | 4.1 |
| 6:1 | 2.9 | 20.3 | 3.6 | 0.8 |

Standard Deviation of maximum release

| | | | | |
|---|---|---|---|---|
| 100:1 | 6.9 | 1.6 | 3.0 | 4.2 |
| 50:1 | 1.6 | 3.0 | 4.7 | 4.2 |
| 25:1 | 6.6 | 6.2 | 3.6 | 3.0 |
| 12.5:1 | 5.7 | 1.6 | 3.8 | 4.8 |
| 6:1 | 5.8 | 5.3 | 3.6 | 4.4 |

### Example 20

### Assays for Macrophage Proliferation and Activity

Functional activity of LP polypeptides can be measured in an appropriately modified macrophage proliferation and cytokine secretion assay. Essentially purified macrophages from the peritoneum, spleen, or liver (1×10⁵) in 200 μL RPMI and 10% FBS media are seeded in triplicate in 96-well plates with different concentrations of LPS in the presence or absence of LP. After 24, 48, or 72 hours, supernatants are collected for cytokine analysis and cells are pulsed for twelve hours with one microcurie of tritiated thymidine. Thymidine incorporation is quantified using a scintillation counter.

### Example 21

### Effect of LP Polypeptides on T Cell Priming and Cytokine Production in Wildtype and LP Transgenic Mice

Functional activity of LP polypeptides can be measured in an appropriately modified T cell priming and cytokine production assay. Essentially, six-week-old WT and LP transgenic mice (four mice in each group) are immunized with 100 μg KLH or HEL in complete Freunds adjuvant (CFA) in the hind footpads. CD4+ T cells are purified from the draining lymph nodes and cultured in the presence of antigen presenting cells (APC), different concentrations of KLH or HEL, and in the presence or absence of LP. APCs are isolated from the spleens of six-week-old WT and LP transgenic mice and irradiated (3,000 rads). T cell recall responses are thereby tested. Cytokine secretion by CD4+ T cells from KLH immunized mice are assayed by ELISA.

To determine if the mechanism of response seen with LP treatment occurs at the level of the APC or intrinsic hyperproliferative response of CD4+ T cells, KLH-primed CD4+ T cells purified from LP transgenic and wild type mice are examined for their recall response in the presence of wild type or LP transgenic APC.

The culture supernatants of T cell recall responses are assayed by ELISA for cytokine production. The production levels of Th1 and Th2 cytokines are observed in order to determine if LP is critically involved in both the Th1 and Th2 differentiation during recall responses.

We claim:

1. Isolated nucleic acid comprising DNA having at least 95% sequence identity to a polynucleotide selected from the group consisting of:
(a) a polynucleotide having a nucleotide sequence as shown in SEQ ID NO:3, 5, or 7;
(b) a polynucleotide having a nucleotide sequence selected from the group consisting of nucleotides 25 or about 109 through about 1419, inclusive, of SEQ ID NO:1, 1 or about 85 through 2103 of SEQ ID NO:3, 25 or about 109 through about 969 of SEQ ID NO:5, and 1 or about 85 through 1461 of SEQ ID NO:7;
(c) a polynucleotide encoding a polypeptide having an amino acid sequence as shown in SEQ ID NO:4, 6, or 8;
(d) a polynucleotide encoding a polypeptide having the amino acid sequence selected from the group consisting of amino acid residues from 1 or about 29 through about 465 of SEQ ID NO:2, 1 or about 29 through 701 of SEQ ID NO:4, and 1 or about 29 through 487 of SEQ ID NO:8;
(e) a polynucleotide fragment of a polynucleotide as in (a), (b), (c), or (d); and
(f) a polynucleotide having a nucleotide sequence which is complementary to the nucleotide sequence of a polynucleotide as in (a), (b), (c), (d), or (e).

2. An isolated nucleic acid molecule encoding a polypeptide comprising DNA that hybridizes to the complement of the nucleic acid sequence that encodes LP276L, LP276ATFV, LP276S, LP276ATFV2, or any fragment or variant thereof.

3. The isolated nucleic acid molecule of claim 2, wherein hybridization occurs under stringent hybridization and wash conditions.

4. A vector comprising the nucleic acid molecule of claim 1.

5. The vector of claim 4, wherein said nucleic acid molecule is operably linked to control sequences recognized by a host cell transformed with the vector.

6. A host cell comprising the vector of claim 5.

7. A process for producing an LP polypeptide comprising culturing the host cell of claim 6 under conditions suitable for expression of said LP polypeptide and recovering said LP polypeptide from the cell culture.

8. An isolated polypeptide comprising an amino acid sequence comprising about 90% sequence identity to a sequence of amino acid residues comprising LP276ATFV, LP276S, LP276ATFV2, as shown in SEQ ID NO: 4, 6, or 8, respectively.

9. An isolated polypeptide comprising an amino acid sequence comprising about 90% sequence identity to a sequence of amino acid residues comprising LP276L, as shown in amino acid residues 29 through about 465 of SEQ ID NO:2.

10. An isolated polypeptide comprising a sequence of amino acid residues selected from the group consisting of:
(a) SEQ ID NO: 4, 6, or 8;
(b) amino acid residues 29 through about 465 of SEQ ID NO:2;
(c) fragments of (a) or (b) sufficient to provide a binding site for an LP polypeptide antibody; and
(d) variants of (a), (b), or (c).

11. An isolated polypeptide produced by the method of claim 7.

12. A chimeric molecule comprising an LP polypeptide fused to a heterologous amino acid sequence.

13. The chimeric molecule of claim 12, wherein said heterologous amino acid sequence is an epitope tag sequence.

14. The chimeric molecule of claim 13, wherein said heterologous amino acid sequence is an Fc region of an immunoglobulin.

15. The chimeric molecule of claim 14 comprising amino acid residues comprising LP276ATFV or LP276ATFV2, as shown in SEQ ID NO:4 or 8, respectively.

16. An antibody which specifically binds to LP276 polypeptide.

17. The antibody of claim 16, wherein said antibody is a monoclonal antibody.

18. The antibody of claim 17, wherein said antibody is selected from the group consisting of a humanized antibody and a human antibody.

19. A composition comprising a therapeutically effective amount of an active agent selected from the group consisting of:
(a) an LP polypeptide;
(b) an agonist to an LP polypeptide;
(c) an antagonist to an LP polypeptide;
(d) an LP polypeptide antibody;
(e) an anti-LP polypeptide-encoding mRNA specific ribozyme; and
(f) a polynucleotide as in claim 1, in combination with a pharmaceutically acceptable carrier.

20. An article of manufacture comprising a container, label and therapeutically effective amount of the composition of claim 19.

21. A method of preventing or treating a disease or pathological condition comprising administering to a patient in need thereof a pharmacologically effective amount of an LP polypeptide.

22. The method of claim 21 wherein the LP polypeptide is selected from the group consisting of LP276 polypeptide, an LP276 analog, a biologically active LP276 polypeptide fragment, an LP276 polypeptide fusion protein, and an LP276 polypeptide isoform.

23. The method of claim 22 wherein the LP276 polypeptide comprises a polypeptide sequence as shown in SEQ ID NO:2 or any biologically active fragment or fusion thereof.

24. The method of claim 21 further comprising administering anti-inflammatory drugs or steroids.

25. The method of claim 21 wherein the LP polypeptide is administered in a single dose.

26. The method of claim 21 wherein the LP polypeptide is administered in multiple doses.

27. The method of claim 21 wherein said disease or pathological condition is selected from the group consisting of sepsis, gram negative bacteremia, inflammation, allergic autoimmune diseases, allergic responses, infectious diseases, immunodeficiencies, type 1 diabetes, Th1-dependent insulitis, pancreatitis, aberrant apoptosis, cancers, rheumatoid arthritis, eczema, psoriasis, atopy, asthma, fibrosing lung disease, acute respiratory distress syndrome (ARDS), inflammatory bowel disease, multiple sclerosis, Hashimoto's thyroiditis, Graves' disease, systemic lupus erythematosis, vasculitis, autoimmune gastritis, HIV, HIV-induced lymphoma, fulminant viral hepatitis B, fulminant viral hepatitis C, chronic hepatitis, chronic cirrhosis, liver failure, chronic glomerulonephritis, thrombotic thrombocytopenic purpura (TTP), hemolytic uremic syndrome (HUS), aplastic anemia, myelodysplasia, transplant rejection, H. pylori associated ulceration, cytoprotection during cancer treatment, recuperation during chemotherapy, recuperation from irradiation therapy, and multiple organ dysfunction syndrome (MODS).

28. The method of claim 21 wherein said disease or condition is a disease or a condition exacerbated by massive neutrophil infiltration.

29. The method according to claim 21 wherein the patient is a mammal.

30. The method according to claims 29 wherein the patient is a human.
